SEQUENCE LISTING (208 sequences)

SEQ ID NO: 1 - protein, 25 aa, synthetic construct
  REGION 1..25: note = PR003181 HFWR1 Chothia, PR004260 HFWR1 Chothia
QVQLQQSGAELVKPGASVKLSCKAS

SEQ ID NO: 2 - protein, 25 aa, synthetic construct
  REGION 1..25: note = PR003248 HFWR1 Chothia, PR003249 HFWR1 Chothia, PR003250 HFWR1 Chothia
QVQLVESGGGVVQPGRSLRLSCAAS

SEQ ID NO: 3 - protein, 25 aa, synthetic construct
  REGION 1..25: note = PR003251 HFWR1 Chothia
QVQLVESGGGVVQPGRSLRLSCAVS

SEQ ID NO: 4 - protein, 25 aa, synthetic construct
  REGION 1..25: note = PR003260 HFWR1 Chothia
QVQLVESGGGVVQPGRSLRLPCAAS

SEQ ID NO: 5 - protein, 25 aa, synthetic construct
  REGION 1..25: note = PR003264 HFWR1 Chothia, PR004263 HFWR1 Chothia
QVQLVQSGAEVKKPGSSVKVSCKTS

SEQ ID NO: 6 - protein, 7 aa, synthetic construct
  REGION 1..7: note = PR003181 HCDR1 Chothia, PR004260 HCDR1 Chothia
GYTFTSH

SEQ ID NO: 7 - protein, 7 aa, synthetic construct
  REGION 1..7: note = PR003248 HCDR1 Chothia, PR003249 HCDR1 Chothia, PR003250 HCDR1 Chothia
GFTFSDY

SEQ ID NO: 8 - protein, 7 aa, synthetic construct
  REGION 1..7: note = PR003251 HCDR1 Chothia, PR003260 HCDR1 Chothia, PR003262 HCDR1 Chothia
GFTFSRY

SEQ ID NO: 9 - protein, 7 aa, synthetic construct
  REGION 1..7: note = PR003252 HCDR1 Chothia, PR003259 HCDR1 Chothia, PR003261 HCDR1 Chothia
GFTFSNY

SEQ ID NO: 10 - protein, 7 aa, synthetic construct
  REGION 1..7: note = PR003254 HCDR1 Chothia
GFTFSSY

SEQ ID NO: 11 - protein, 7 aa, synthetic construct
  REGION 1..7: note = PR003263 HCDR1 Chothia
GFTFSYY

SEQ ID NO: 12 - protein, 7 aa, synthetic construct
  REGION 1..7: note = PR003264 HCDR1 Chothia, PR004263 HCDR1 Chothia
GGTFSSY

SEQ ID NO: 13 - protein, 7 aa, synthetic construct
  REGION 1..7: note = PR003265 HCDR1 Chothia
GFTFSTY

SEQ ID NO: 14 - protein, 19 aa, synthetic construct
  REGION 1..19: note = PR003181 HFWR2 Chothia, PR004260 HFWR2 Chothia
DINWVRQRPEQGLEWIGWI

SEQ ID NO: 15 - protein, 19 aa, synthetic construct
  REGION 1..19: note = PR003248 HFWR2 Chothia
GLHWVRQAPGRGLEWVAVI

SEQ ID NO: 16 - protein, 19 aa, synthetic construct
  REGION 1..19: note = PR003249 HFWR2 Chothia, PR003250 HFWR2 Chothia, PR003262 HFWR2 Chothia
GIHWVRQAPGKGLEWVAVI

SEQ ID NO: 17 - protein, 19 aa, synthetic construct
  REGION 1..19: note = PR003251 HFWR2 Chothia
GMHWVRQAPGKGLDWVAVI

SEQ ID NO: 18 - protein, 19 aa, synthetic construct
  REGION 1..19: note = PR003252 HFWR2 Chothia
AMHWVRQVPGKGLEWVALI

SEQ ID NO: 19 - protein, 19 aa, synthetic construct
  REGION 1..19: note = PR003254 HFWR2 Chothia, PR003260 HFWR2 Chothia
GMHWVRQAPGKGLEWVADI

SEQ ID NO: 20 - protein, 19 aa, synthetic construct
  REGION 1..19: note = PR003259 HFWR2 Chothia, PR004262 HFWR2 Chothia, PR004487 HFWR2 Chothia
GMHWVRQVPGKGLEWVAVI

SEQ ID NO: 21 - protein, 19 aa, synthetic construct
  REGION 1..19: note = PR003261 HFWR2 Chothia
GMHWVRQAPGKGLEWVAVI

SEQ ID NO: 22 - protein, 19 aa, synthetic construct
  REGION 1..19: note = PR003263 HFWR2 Chothia
GLNWVRQAPGKGLEWVAVI

SEQ ID NO: 23 - protein, 19 aa, synthetic construct
  REGION 1..19: note = PR003264 HFWR2 Chothia, PR004263 HFWR2 Chothia
AISWVRQAPGQGLEWMGGI

SEQ ID NO: 24 - protein, 19 aa, synthetic construct
  REGION 1..19: note = PR003265 HFWR2 Chothia
GLHWVRQAPGKGLEWVAVI

SEQ ID NO: 25 - protein, 19 aa, synthetic construct
  REGION 1..19: note = PR003266 HFWR2 Chothia
GMHWVRQAPGKGLEWVAII

SEQ ID NO: 26 - protein, 6 aa, synthetic construct
  REGION 1..6: note = PR003181 HCDR2 Chothia, PR004260 HCDR2 Chothia
FPGDGS

SEQ ID NO: 27 - protein, 6 aa, synthetic construct
  REGION 1..6: note = PR003248 HCDR2 Chothia, PR003252 HCDR2 Chothia, PR003259 HCDR2 Chothia
WYDGSE

SEQ ID NO: 28 - protein, 6 aa, synthetic construct
  REGION 1..6: note = PR003249 HCDR2 Chothia, PR003250 HCDR2 Chothia, PR004261 HCDR2 Chothia
WYDGSD

SEQ ID NO: 29 - protein, 6 aa, synthetic construct
  REGION 1..6: note = PR003251 HCDR2 Chothia, PR003254 HCDR2 Chothia
WYNGNN

SEQ ID NO: 30 - protein, 6 aa, synthetic construct
  REGION 1..6: note = PR003260 HCDR2 Chothia
WYDGSK

SEQ ID NO: 31 - protein, 6 aa, synthetic construct
  REGION 1..6: note = PR003261 HCDR2 Chothia
WYDGTN

SEQ ID NO: 32 - protein, 6 aa, synthetic construct
  REGION 1..6: note = PR003262 HCDR2 Chothia
WYNGRN

SEQ ID NO: 33 - protein, 6 aa, synthetic construct
  REGION 1..6: note = PR003263 HCDR2 Chothia
WFDGTN

SEQ ID NO: 34 - protein, 6 aa, synthetic construct
  REGION 1..6: note = PR003264 HCDR2 Chothia, PR004263 HCDR2 Chothia
IPLIGT

SEQ ID NO: 35 - protein, 6 aa, synthetic construct
  REGION 1..6: note = PR003265 HCDR2 Chothia
WYDESN

SEQ ID NO: 36 - protein, 6 aa, synthetic construct
  REGION 1..6: note = PR003266 HCDR2 Chothia
WYDGSN

SEQ ID NO: 37 - protein, 6 aa, synthetic construct
  REGION 1..6: note = PR004479 HCDR2 Chothia, PR004480 HCDR2 Chothia, PR004481 HCDR2 Chothia
WYEGSD

SEQ ID NO: 38 - protein, 6 aa, synthetic construct
  REGION 1..6: note = PR004482 HCDR2 Chothia, PR004483 HCDR2 Chothia, PR004484 HCDR2 Chothia
WYDASD

SEQ ID NO: 39 - protein, 6 aa, synthetic construct
  REGION 1..6: note = PR004487 HCDR2 Chothia, PR004488 HCDR2 Chothia, PR004489 HCDR2 Chothia
WYEGSE

SEQ ID NO: 40 - protein, 6 aa, synthetic construct
  REGION 1..6: note = PR004490 HCDR2 Chothia, PR004491 HCDR2 Chothia, PR004492 HCDR2 Chothia
WYDASE

SEQ ID NO: 41 - protein, 41 aa, synthetic construct
  REGION 1..41: note = PR003181 HFWR3 Chothia, PR004260 HFWR3 Chothia
TKFNEKFKGKATLTTDKSSSTAYIQLSRLTSEDSAVYFCAR

SEQ ID NO: 42 - protein, 41 aa, synthetic construct
  REGION 1..41: note = PR003248 HFWR3 Chothia
EYYADSVKGRFTISRDNSKKSLYLQMNSLRAEDTAVYFCAR

SEQ ID NO: 43 - protein, 41 aa, synthetic construct
  REGION 1..41: note = PR003249 HFWR3 Chothia, PR004261 HFWR3 Chothia, PR004479 HFWR3 Chothia
KYYADSVRGRFTISRDNSKNTLSLQMNSLRVEDTAVYYCAR

SEQ ID NO: 44 - protein, 41 aa, synthetic construct
  REGION 1..41: note = PR003250 HFWR3 Chothia
KYYADSVRGRFTISRDNSKNTLSLQMNSLRAEDTAVYYCAR

SEQ ID NO: 45 - protein, 41 aa, synthetic construct
  REGION 1..41: note = PR003251 HFWR3 Chothia
KYYTDSVKGRFTISRDNSKNTLFLQMNSLRAEDTAVYYCAR

SEQ ID NO: 46 - protein, 41 aa, synthetic construct
  REGION 1..41: note = PR003252 HFWR3 Chothia
KYYADSVKGRFTISRDDSKNTLFLQMSSLRAEDTAVYSCVR

SEQ ID NO: 47 - protein, 41 aa, synthetic construct
  REGION 1..41: note = PR003254 HFWR3 Chothia
KYYADSVKGRFTISRDNSKNTLYLQMNSLRAEDTALYYCAR

SEQ ID NO: 48 - protein, 41 aa, synthetic construct
  REGION 1..41: note = PR003259 HFWR3 Chothia, PR004262 HFWR3 Chothia, PR004487 HFWR3 Chothia
KDYADSVKGRFTISRDNSKNTLYLQMNSLRVEDTAVYYCAK

SEQ ID NO: 49 - protein, 41 aa, synthetic construct
  REGION 1..41: note = PR003260 HFWR3 Chothia
KYYADSVKGRFTISRDNSKNTLYLQMNSLRAEDTAVYFCTR

SEQ ID NO: 50 - protein, 41 aa, synthetic construct
  REGION 1..41: note = PR003261 HFWR3 Chothia
KNYADSVKGRFTISRDNSKNTLDLQMNSLRAEDTAVYFCAR

SEQ ID NO: 51 - protein, 41 aa, synthetic construct
  REGION 1..41: note = PR003262 HFWR3 Chothia
KYYVDSVKGRFTISRDNSKNTLYLQMNSLRAEDTAVYYCAR

SEQ ID NO: 52 - protein, 41 aa, synthetic construct
  REGION 1..41: note = PR003263 HFWR3 Chothia
KYYADSVKGRFTISRDDSKNTLYLQMNSLRAEDTAVYYCAR

SEQ ID NO: 53 - protein, 41 aa, synthetic construct
  REGION 1..41: note = PR003264 HFWR3 Chothia, PR004263 HFWR3 Chothia
ANYAQNFQGRVTITADESTRTAYMELSSLRSDDTAVYYCAR

SEQ ID NO: 54 - protein, 41 aa, synthetic construct
  REGION 1..41: note = PR003265 HFWR3 Chothia
EYYADSVKGRFTISRDNSKNTLYLQMNSLRAEDTALYYCAR

SEQ ID NO: 55 - protein, 41 aa, synthetic construct
  REGION 1..41: note = PR003266 HFWR3 Chothia
KYYADSVKGRFTISRDNSKNTLYLQMNSLRAEDTAVYYCAR

SEQ ID NO: 56 - protein, 8 aa, synthetic construct
  REGION 1..8: note = PR003181 HCDR3 Chothia, PR004260 HCDR3 Chothia
NSFYSMDY

SEQ ID NO: 57 - protein, 12 aa, synthetic construct
  REGION 1..12: note = PR003248 HCDR3 Chothia
DRNSGALGYFPH

SEQ ID NO: 58 - protein, 12 aa, synthetic construct
  REGION 1..12: note = PR003249 HCDR3 Chothia, PR004261 HCDR3 Chothia, PR004479 HCDR3 Chothia
DRSSGAMGYFVY

SEQ ID NO: 59 - protein, 12 aa, synthetic construct
  REGION 1..12: note = PR003250 HCDR3 Chothia
DRSSGAMGYFAY

SEQ ID NO: 60 - protein, 11 aa, synthetic construct
  REGION 1..11: note = PR003251 HCDR3 Chothia
DGAYSYGPFDY

SEQ ID NO: 61 - protein, 11 aa, synthetic construct
  REGION 1..11: note = PR003252 HCDR3 Chothia
EYSSGWGYFDY

SEQ ID NO: 62 - protein, 11 aa, synthetic construct
  REGION 1..11: note = PR003254 HCDR3 Chothia, PR003260 HCDR3 Chothia, PR003262 HCDR3 Chothia
DGGYSYGPFDY

SEQ ID NO: 63 - protein, 11 aa, synthetic construct
  REGION 1..11: note = PR003259 HCDR3 Chothia, PR004262 HCDR3 Chothia, PR004487 HCDR3 Chothia
DLISGIYYVDH

SEQ ID NO: 64 - protein, 11 aa, synthetic construct
  REGION 1..11: note = PR003261 HCDR3 Chothia
DRNTAMDYFVY

SEQ ID NO: 65 - protein, 14 aa, synthetic construct
  REGION 1..14: note = PR003263 HCDR3 Chothia
GYGSGSYYNYVMDA

SEQ ID NO: 66 - protein, 12 aa, synthetic construct
  REGION 1..12: note = PR003264 HCDR3 Chothia, PR004263 HCDR3 Chothia
DTMVRGVRALDY

SEQ ID NO: 67 - protein, 12 aa, synthetic construct
  REGION 1..12: note = PR003265 HCDR3 Chothia
LAHDYGGTYFDY

SEQ ID NO: 68 - protein, 12 aa, synthetic construct
  REGION 1..12: note = PR003266 HCDR3 Chothia
DRSSGALGYFQH

SEQ ID NO: 69 - protein, 11 aa, synthetic construct
  REGION 1..11: note = PR003181 HFWR4 Chothia, PR004260 HFWR4 Chothia
WGQGTSVTVSS

SEQ ID NO: 70 - protein, 11 aa, synthetic construct
  REGION 1..11: note = PR003248 HFWR4 Chothia, PR003249 HFWR4 Chothia, PR003250 HFWR4 Chothia
WGQGTLVTVSS

SEQ ID NO: 71 - protein, 11 aa, synthetic construct
  REGION 1..11: note = PR003252 HFWR4 Chothia
WGQGTQVTVSS

SEQ ID NO: 72 - protein, 11 aa, synthetic construct
  REGION 1..11: note = PR003263 HFWR4 Chothia
WGQGASVTVSS

SEQ ID NO: 73 - protein, 23 aa, synthetic construct
  REGION 1..23: note = PR003181 LFWR1 Chothia, PR004260 LFWR1 Chothia
DIVMSQSPSSLAVSAGEKVTMSC

SEQ ID NO: 74 - protein, 23 aa, synthetic construct
  REGION 1..23: note = PR003248 LFWR1 Chothia, PR003249 LFWR1 Chothia, PR003250 LFWR1 Chothia
DIQMTQSPSTLSASVGDRVTITC

SEQ ID NO: 75 - protein, 23 aa, synthetic construct
  REGION 1..23: note = PR003251 LFWR1 Chothia
EIVMTQSPATLSVSPGEGATLSC

SEQ ID NO: 76 - protein, 23 aa, synthetic construct
  REGION 1..23: note = PR003254 LFWR1 Chothia, PR003260 LFWR1 Chothia, PR003262 LFWR1 Chothia
EIVMTQSPATLSVSPGERATLSC

SEQ ID NO: 77 - protein, 23 aa, synthetic construct
  REGION 1..23: note = PR003259 LFWR1 Chothia, PR004262 LFWR1 Chothia, PR004487 LFWR1 Chothia
DIQMTQSPSSLSASVGDRVTITC

SEQ ID NO: 78 - protein, 23 aa, synthetic construct
  REGION 1..23: note = PR003264 LFWR1 Chothia, PR004263 LFWR1 Chothia
DIQLTQSPSFLSASVGDRVTITC

SEQ ID NO: 79 - protein, 23 aa, synthetic construct
  REGION 1..23: note = PR003265 LFWR1 Chothia
EIVMTQSPAILSVSPGERATLSC

SEQ ID NO: 80 - protein, 17 aa, synthetic construct
  REGION 1..17: note = PR003181 LCDR1 Chothia, PR004260 LCDR1 Chothia
KSSQSLLNSRTRKNQLA

SEQ ID NO: 81 - protein, 11 aa, synthetic construct
  REGION 1..11: note = PR003248 LCDR1 Chothia
RASQSINNWLA

SEQ ID NO: 82 - protein, 11 aa, synthetic construct
  REGION 1..11: note = PR003249 LCDR1 Chothia, PR003250 LCDR1 Chothia, PR003252 LCDR1 Chothia
RASQSISSWLA

SEQ ID NO: 83 - protein, 11 aa, synthetic construct
  REGION 1..11: note = PR003251 LCDR1 Chothia, PR003260 LCDR1 Chothia, PR003263 LCDR1 Chothia
RASQSVSSNLA

SEQ ID NO: 84 - protein, 11 aa, synthetic construct
  REGION 1..11: note = PR003254 LCDR1 Chothia
RASQSVSSYLA

SEQ ID NO: 85 - protein, 11 aa, synthetic construct
  REGION 1..11: note = PR003259 LCDR1 Chothia, PR004262 LCDR1 Chothia, PR004487 LCDR1 Chothia
RASQGISNYLA

SEQ ID NO: 86 - protein, 11 aa, synthetic construct
  REGION 1..11: note = PR003262 LCDR1 Chothia
RASQSFSNNLA

SEQ ID NO: 87 - protein, 11 aa, synthetic construct
  REGION 1..11: note = PR003264 LCDR1 Chothia, PR004263 LCDR1 Chothia
RASQGISSYFA

SEQ ID NO: 88 - protein, 11 aa, synthetic construct
  REGION 1..11: note = PR003265 LCDR1 Chothia
RASQSISYNLA

SEQ ID NO: 89 - protein, 15 aa, synthetic construct
  REGION 1..15: note = PR003181 LFWR2 Chothia, PR004260 LFWR2 Chothia
WYQQKPGQSPKLLIY

SEQ ID NO: 90 - protein, 15 aa, synthetic construct
  REGION 1..15: note = PR003248 LFWR2 Chothia, PR003249 LFWR2 Chothia, PR003250 LFWR2 Chothia
WYQQKPGKAPKLLIY

SEQ ID NO: 91 - protein, 15 aa, synthetic construct
  REGION 1..15: note = PR003251 LFWR2 Chothia, PR003254 LFWR2 Chothia, PR003260 LFWR2 Chothia
WYQQKPGQAPRLLIY

SEQ ID NO: 92 - protein, 15 aa, synthetic construct
  REGION 1..15: note = PR003259 LFWR2 Chothia, PR004262 LFWR2 Chothia, PR004487 LFWR2 Chothia
WYQQKPGKVPKLLIY

SEQ ID NO: 93 - protein, 7 aa, synthetic construct
  REGION 1..7: note = PR003181 LCDR2 Chothia, PR004260 LCDR2 Chothia
WASTRES

SEQ ID NO: 94 - protein, 7 aa, synthetic construct
  REGION 1..7: note = PR003248 LCDR2 Chothia, PR003249 LCDR2 Chothia, PR003250 LCDR2 Chothia
KASSLES

SEQ ID NO: 95 - protein, 7 aa, synthetic construct
  REGION 1..7: note = PR003251 LCDR2 Chothia, PR003254 LCDR2 Chothia, PR003262 LCDR2 Chothia
GASTRAT

SEQ ID NO: 96 - protein, 7 aa, synthetic construct
  REGION 1..7: note = PR003252 LCDR2 Chothia
KASSLKS

SEQ ID NO: 97 - protein, 7 aa, synthetic construct
  REGION 1..7: note = PR003259 LCDR2 Chothia, PR003264 LCDR2 Chothia, PR004262 LCDR2 Chothia
AASTLQS

SEQ ID NO: 98 - protein, 7 aa, synthetic construct
  REGION 1..7: note = PR003260 LCDR2 Chothia
YASTRAP

SEQ ID NO: 99 - protein, 7 aa, synthetic construct
  REGION 1..7: note = PR003263 LCDR2 Chothia
GASTGAT

SEQ ID NO: 100 - protein, 7 aa, synthetic construct
  REGION 1..7: note = PR003265 LCDR2 Chothia
GASIRAT

SEQ ID NO: 101 - protein, 32 aa, synthetic construct
  REGION 1..32: note = PR003181 LFWR3 Chothia, PR004260 LFWR3 Chothia
GVPDRFTGSGSGTDFTLTISSVQAEDLAVYYC

SEQ ID NO: 102 - protein, 32 aa, synthetic construct
  REGION 1..32: note = PR003248 LFWR3 Chothia, PR003252 LFWR3 Chothia, PR003266 LFWR3 Chothia
GVPSRFSGSGSGTEFTLTISSLQPDDFATYYC

SEQ ID NO: 103 - protein, 32 aa, synthetic construct
  REGION 1..32: note = PR003249 LFWR3 Chothia, PR003250 LFWR3 Chothia, PR004261 LFWR3 Chothia
GVPSRFSGSGSGTEFTLTISSLQPDDLATYYC

SEQ ID NO: 104 - protein, 32 aa, synthetic construct
  REGION 1..32: note = PR003251 LFWR3 Chothia, PR003254 LFWR3 Chothia, PR003262 LFWR3 Chothia
GIPARFSGSGSGTEFTLTISSLQSEDFAVYYC

SEQ ID NO: 105 - protein, 32 aa, synthetic construct
  REGION 1..32: note = PR003259 LFWR3 Chothia, PR004262 LFWR3 Chothia, PR004487 LFWR3 Chothia
GVPSRFSGSGSGTDFTLTISSLQPEDVATYYC

SEQ ID NO: 106 - protein, 32 aa, synthetic construct
  REGION 1..32: note = PR003260 LFWR3 Chothia
GIPARFSGSESGTEFTLTISSLQSEDFAVYYC

SEQ ID NO: 107 - protein, 32 aa, synthetic construct
  REGION 1..32: note = PR003261 LFWR3 Chothia
GVPSRFSGSGSGTEFTLTISSLQPDDFASYYC

SEQ ID NO: 108 - protein, 32 aa, synthetic construct
  REGION 1..32: note = PR003263 LFWR3 Chothia, PR003265 LFWR3 Chothia
GIPARFSGSGSGTDFTLTISSLQSEDFAVYYC

SEQ ID NO: 109 - protein, 32 aa, synthetic construct
  REGION 1..32: note = PR003264 LFWR3 Chothia, PR004263 LFWR3 Chothia
GVPSRFSGSGSGTEFTLTISSLQPEDFATYYC

SEQ ID NO: 110 - protein, 8 aa, synthetic construct
  REGION 1..8: note = PR003181 LCDR3 Chothia, PR004260 LCDR3 Chothia
KQSYNLRT

SEQ ID NO: 111 - protein, 8 aa, synthetic construct
  REGION 1..8: note = PR003248 LCDR3 Chothia, PR003266 LCDR3 Chothia
QQYNSYST

SEQ ID NO: 112 - protein, 9 aa, synthetic construct
  REGION 1..9: note = PR003249 LCDR3 Chothia, PR003250 LCDR3 Chothia, PR004261 LCDR3 Chothia
QQYNSYSYT

SEQ ID NO: 113 - protein, 9 aa, synthetic construct
  REGION 1..9: note = PR003251 LCDR3 Chothia, PR003260 LCDR3 Chothia
QQYNIWPYT

SEQ ID NO: 114 - protein, 8 aa, synthetic construct
  REGION 1..8: note = PR003252 LCDR3 Chothia
LQYNSYYT

SEQ ID NO: 115 - protein, 9 aa, synthetic construct
  REGION 1..9: note = PR003254 LCDR3 Chothia
QQYNNWPLT

SEQ ID NO: 116 - protein, 9 aa, synthetic construct
  REGION 1..9: note = PR003259 LCDR3 Chothia, PR004262 LCDR3 Chothia, PR004487 LCDR3 Chothia
QKYNSAPFT

SEQ ID NO: 117 - protein, 9 aa, synthetic construct
  REGION 1..9: note = PR003261 LCDR3 Chothia
QQYNSYSVT

SEQ ID NO: 118 - protein, 9 aa, synthetic construct
  REGION 1..9: note = PR003262 LCDR3 Chothia
HQYNIWPLT

SEQ ID NO: 119 - protein, 9 aa, synthetic construct
  REGION 1..9: note = PR003263 LCDR3 Chothia
QQYYKWPLT

SEQ ID NO: 120 - protein, 9 aa, synthetic construct
  REGION 1..9: note = PR003264 LCDR3 Chothia, PR004263 LCDR3 Chothia
QHLNRYPWT

SEQ ID NO: 121 - protein, 10 aa, synthetic construct [sequence omitted]

SEQ ID NO: 122 - protein, 9 aa, synthetic construct
  REGION 1..9: note = PR004480 LCDR3 Chothia, PR004483 LCDR3 Chothia, PR004485 LCDR3 Chothia
QQYQSYSYT

SEQ ID NO: 123 - protein, 9 aa, synthetic construct
  REGION 1..9: note = PR004481 LCDR3 Chothia, PR004484 LCDR3 Chothia, PR004486 LCDR3 Chothia
QQYNAYSYT

SEQ ID NO: 124 - protein, 9 aa, synthetic construct
  REGION 1..9: note = PR004488 LCDR3 Chothia, PR004491 LCDR3 Chothia, PR004493 LCDR3 Chothia
QKYQSAPFT

SEQ ID NO: 125 - protein, 9 aa, synthetic construct
  REGION 1..9: note = PR004489 LCDR3 Chothia, PR004492 LCDR3 Chothia, PR004494 LCDR3 Chothia
QKYNAAPFT

SEQ ID NO: 126 - protein, 10 aa, synthetic construct
  REGION 1..10: note = PR003181 LFWR4 Chothia, PR004260 LFWR4 Chothia
FGGGTKLEIK

SEQ ID NO: 127 - protein, 10 aa, synthetic construct
  REGION 1..10: note = PR003248 LFWR4 Chothia, PR003249 LFWR4 Chothia, PR003250 LFWR4 Chothia
FGQGTKLEIK

SEQ ID NO: 128 - protein, 10 aa, synthetic construct
  REGION 1..10: note = PR003254 LFWR4 Chothia, PR003261 LFWR4 Chothia, PR003262 LFWR4 Chothia
FGGGTKVEIK

SEQ ID NO: 129 - protein, 10 aa, synthetic construct
  REGION 1..10: note = PR003259 LFWR4 Chothia, PR004262 LFWR4 Chothia, PR004487 LFWR4 Chothia
FGPGTKVDIK

SEQ ID NO: 130 - protein, 10 aa, synthetic construct
  REGION 1..10: note = PR003264 LFWR4 Chothia, PR003265 LFWR4 Chothia, PR004263 LFWR4 Chothia
FGQGTKVEIK

SEQ ID NO: 131 - protein, 117 aa, synthetic construct
  REGION 1..117: note = PR003181 VH, PR004260 VH
QVQLQQSGAELVKPGASVKLSCKASGYTFTSHDINWVRQRPEQGLEWIGWIFPGDGSTKFNEKFKGKATLTTDKSSSTAYIQLSRLTSEDSAVYFCARNSFYSMDYWGQGTSVTVSS

SEQ ID NO: 132 - protein, 121 aa, synthetic construct
  REGION 1..121: note = PR003248 VH
QVQLVESGGGVVQPGRSLRLSCAASGFTFSDYGLHWVRQAPGRGLEWVAVIWYDGSEEYYADSVKGRFTISRDNSKKSLYLQMNSLRAEDTAVYFCARDRNSGALGYFPHWGQGTLVTVSS

SEQ ID NO: 133 - protein, 121 aa, synthetic construct
  REGION 1..121: note = PR003249 VH, PR004261 VH, PR004485 VH, PR004486 VH
QVQLVESGGGVVQPGRSLRLSCAASGFTFSDYGIHWVRQAPGKGLEWVAVIWYDGSDKYYADSVRGRFTISRDNSKNTLSLQMNSLRVEDTAVYYCARDRSSGAMGYFVYWGQGTLVTVSS

SEQ ID NO: 134 - protein, 121 aa, synthetic construct
  REGION 1..121: note = PR003250 VH
QVQLVESGGGVVQPGRSLRLSCAASGFTFSDYGIHWVRQAPGKGLEWVAVIWYDGSDKYYADSVRGRFTISRDNSKNTLSLQMNSLRAEDTAVYYCARDRSSGAMGYFAYWGQGTLVTVSS

SEQ ID NO: 135 - protein, 120 aa, synthetic construct
  REGION 1..120: note = PR003251 VH
QVQLVESGGGVVQPGRSLRLSCAVSGFTFSRYGMHWVRQAPGKGLDWVAVIWYNGNNKYYTDSVKGRFTISRDNSKNTLFLQMNSLRAEDTAVYYCARDGAYSYGPFDYWGQGTLVTVSS

SEQ ID NO: 136 - protein, 120 aa, synthetic construct
  REGION 1..120: note = PR003252 VH
QVQLVESGGGVVQPGRSLRLSCAASGFTFSNYAMHWVRQVPGKGLEWVALIWYDGSEKYYADSVKGRFTISRDDSKNTLFLQMSSLRAEDTAVYSCVREYSSGWGYFDYWGQGTQVTVSS

SEQ ID NO: 137 - protein, 120 aa, synthetic construct
  REGION 1..120: note = PR003254 VH
QVQLVESGGGVVQPGRSLRLSCAASGFTFSSYGMHWVRQAPGKGLEWVADIWYNGNNKYYADSVKGRFTISRDNSKNTLYLQMNSLRAEDTALYYCARDGGYSYGPFDYWGQGTLVTVSS

SEQ ID NO: 138 - protein, 120 aa, synthetic construct
  REGION 1..120: note = PR003259 VH, PR004262 VH, PR004493 VH, PR004494 VH
QVQLVESGGGVVQPGRSLRLSCAASGFTFSNYGMHWVRQVPGKGLEWVAVIWYDGSEKDYADSVKGRFTISRDNSKNTLYLQMNSLRVEDTAVYYCAKDLISGIYYVDHWGQGTLVTVSS

SEQ ID NO: 139 - protein, 120 aa, synthetic construct
  REGION 1..120: note = PR003260 VH
QVQLVESGGGVVQPGRSLRLPCAASGFTFSRYGMHWVRQAPGKGLEWVADIWYDGSKKYYADSVKGRFTISRDNSKNTLYLQMNSLRAEDTAVYFCTRDGGYSYGPFDYWGQGTLVTVSS

SEQ ID NO: 140 - protein, 120 aa, synthetic construct
  REGION 1..120: note = PR003261 VH
QVQLVESGGGVVQPGRSLRLSCAASGFTFSNYGMHWVRQAPGKGLEWVAVIWYDGTNKNYADSVKGRFTISRDNSKNTLDLQMNSLRAEDTAVYFCARDRNTAMDYFVYWGQGTLVTVSS

SEQ ID NO: 141 - protein, 120 aa, synthetic construct
  REGION 1..120: note = PR003262 VH
QVQLVESGGGVVQPGRSLRLSCAASGFTFSRYGIHWVRQAPGKGLEWVAVIWYNGRNKYYVDSVKGRFTISRDNSKNTLYLQMNSLRAEDTAVYYCARDGGYSYGPFDYWGQGTLVTVSS

SEQ ID NO: 142 - protein, 123 aa, synthetic construct
  REGION 1..123: note = PR003263 VH
QVQLVESGGGVVQPGRSLRLSCAASGFTFSYYGLNWVRQAPGKGLEWVAVIWFDGTNKYYADSVKGRFTISRDDSKNTLYLQMNSLRAEDTAVYYCARGYGSGSYYNYVMDAWGQGASVTVSS

SEQ ID NO: 143 - protein, 121 aa, synthetic construct
  REGION 1..121: note = PR003264 VH, PR004263 VH
QVQLVQSGAEVKKPGSSVKVSCKTSGGTFSSYAISWVRQAPGQGLEWMGGIIPLIGTANYAQNFQGRVTITADESTRTAYMELSSLRSDDTAVYYCARDTMVRGVRALDYWGQGTLVTVSS

SEQ ID NO: 144 - protein, 121 aa, synthetic construct
  REGION 1..121: note = PR003265 VH
QVQLVESGGGVVQPGRSLRLSCAASGFTFSTYGLHWVRQAPGKGLEWVAVIWYDESNEYYADSVKGRFTISRDNSKNTLYLQMNSLRAEDTALYYCARLAHDYGGTYFDYWGQGTLVTVSS

SEQ ID NO: 145 - protein, 121 aa, synthetic construct
  REGION 1..121: note = PR003266 VH
QVQLVESGGGVVQPGRSLRLSCAASGFTFSDYGMHWVRQAPGKGLEWVAIIWYDGSNKYYADSVKGRFTISRDNSKNTLYLQMNSLRAEDTAVYYCARDRSSGALGYFQHWGQGTLVTVSS

SEQ ID NO: 146 - protein, 121 aa, synthetic construct
  REGION 1..121: note = PR004479 VH, PR004480 VH, PR004481 VH
QVQLVESGGGVVQPGRSLRLSCAASGFTFSDYGIHWVRQAPGKGLEWVAVIWYEGSDKYYADSVRGRFTISRDNSKNTLSLQMNSLRVEDTAVYYCARDRSSGAMGYFVYWGQGTLVTVSS

SEQ ID NO: 147 - protein, 121 aa, synthetic construct
  REGION 1..121: note = PR004482 VH, PR004483 VH, PR004484 VH
QVQLVESGGGVVQPGRSLRLSCAASGFTFSDYGIHWVRQAPGKGLEWVAVIWYDASDKYYADSVRGRFTISRDNSKNTLSLQMNSLRVEDTAVYYCARDRSSGAMGYFVYWGQGTLVTVSS

SEQ ID NO: 148 - protein, 120 aa, synthetic construct
  REGION 1..120: note = PR004487 VH, PR004488 VH, PR004489 VH
QVQLVESGGGVVQPGRSLRLSCAASGFTFSNYGMHWVRQVPGKGLEWVAVIWYEGSEKDYADSVKGRFTISRDNSKNTLYLQMNSLRVEDTAVYYCAKDLISGIYYVDHWGQGTLVTVSS

SEQ ID NO: 149 - protein, 120 aa, synthetic construct
  REGION 1..120: note = PR004490 VH, PR004491 VH, PR004492 VH
QVQLVESGGGVVQPGRSLRLSCAASGFTFSNYGMHWVRQVPGKGLEWVAVIWYDASEKDYADSVKGRFTISRDNSKNTLYLQMNSLRVEDTAVYYCAKDLISGIYYVDHWGQGTLVTVSS

SEQ ID NO: 150 - protein, 112 aa, synthetic construct
  REGION 1..112: note = PR003181 VL, PR004260 VL
DIVMSQSPSSLAVSAGEKVTMSCKSSQSLLNSRTRKNQLAWYQQKPGQSPKLLIYWASTRESGVPDRFTGSGSGTDFTLTISSVQAEDLAVYYCKQSYNLRTFGGGTKLEIK

SEQ ID NO: 151 - protein, 106 aa, synthetic construct
  REGION 1..106: note = PR003248 VL
DIQMTQSPSTLSASVGDRVTITCRASQSINNWLAWYQQKPGKAPKLLIYKASSLESGVPSRFSGSGSGTEFTLTISSLQPDDFATYYCQQYNSYSTFGQGTKLEIK

SEQ ID NO: 152 - protein, 107 aa, synthetic construct
  REGION 1..107: note = PR003249 VL, PR003250 VL, PR004261 VL, PR004479 VL, PR004482 VL
DIQMTQSPSTLSASVGDRVTITCRASQSISSWLAWYQQKPGKAPKLLIYKASSLESGVPSRFSGSGSGTEFTLTISSLQPDDLATYYCQQYNSYSYTFGQGTKLEIK

SEQ ID NO: 153 - protein, 107 aa, synthetic construct
  REGION 1..107: note = PR003251 VL
EIVMTQSPATLSVSPGEGATLSCRASQSVSSNLAWYQQKPGQAPRLLIYGASTRATGIPARFSGSGSGTEFTLTISSLQSEDFAVYYCQQYNIWPYTFGQGTKLEIK

SEQ ID NO: 154 - protein, 106 aa, synthetic construct
  REGION 1..106: note = PR003252 VL
DIQMTQSPSTLSASVGDRVTITCRASQSISSWLAWYQQKPGKAPKLLIYKASSLKSGVPSRFSGSGSGTEFTLTISSLQPDDFATYYCLQYNSYYTFGQGTKLEIK

SEQ ID NO: 155 - protein, 107 aa, synthetic construct
  REGION 1..107: note = PR003254 VL
EIVMTQSPATLSVSPGERATLSCRASQSVSSYLAWYQQKPGQAPRLLIYGASTRATGIPARFSGSGSGTEFTLTISSLQSEDFAVYYCQQYNNWPLTFGGGTKVEIK

SEQ ID NO: 156 - protein, 107 aa, synthetic construct
  REGION 1..107: note = PR003259 VL, PR004262 VL, PR004487 VL, PR004490 VL
DIQMTQSPSSLSASVGDRVTITCRASQGISNYLAWYQQKPGKVPKLLIYAASTLQSGVPSRFSGSGSGTDFTLTISSLQPEDVATYYCQKYNSAPFTFGPGTKVDIK

SEQ ID NO: 157 - protein, 107 aa, synthetic construct
  REGION 1..107: note = PR003260 VL
EIVMTQSPATLSVSPGERATLSCRASQSVSSNLAWYQQKPGQAPRLLIYYASTRAPGIPARFSGSESGTEFTLTISSLQSEDFAVYYCQQYNIWPYTFGQGTKLEIK

SEQ ID NO: 158 - protein, 107 aa, synthetic construct
  REGION 1..107: note = PR003261 VL
DIQMTQSPSTLSASVGDRVTITCRASQSISSWLAWYQQKPGKAPKLLIYKASSLESGVPSRFSGSGSGTEFTLTISSLQPDDFASYYCQQYNSYSVTFGGGTKVEIK

SEQ ID NO: 159 - protein, 107 aa, synthetic construct
  REGION 1..107: note = PR003262 VL
EIVMTQSPATLSVSPGERATLSCRASQSFSNNLAWYQQKPGQAPRLLIYGASTRATGIPARFSGSGSGTEFTLTISSLQSEDFAVYYCHQYNIWPLTFGGGTKVEIK

SEQ ID NO: 160 - protein, 107 aa, synthetic construct
  REGION 1..107: note = PR003263 VL
EIVMTQSPATLSVSPGERATLSCRASQSVSSNLAWYQQKPGQAPRLLIYGASTGATGIPARFSGSGSGTDFTLTISSLQSEDFAVYYCQQYYKWPLTFGGGTKVEIK

SEQ ID NO: 161 - protein, 107 aa, synthetic construct
  REGION 1..107: note = PR003264 VL, PR004263 VL
DIQLTQSPSFLSASVGDRVTITCRASQGISSYFAWYQQKPGKAPKLLIYAASTLQSGVPSRFSGSGSGTEFTLTISSLQPEDFATYYCQHLNRYPWTFGQGTKVEIK

SEQ ID NO: 162 - protein, 108 aa, synthetic construct
  REGION 1..108: note = PR003265 VL
EIVMTQSPAILSVSPGERATLSCRASQSISYNLAWYQQKPGQAPRLLIYGASIRATGIPARFSGSGSGTDFTLTISSLQSEDFAVYYCQQYNNWPPWTFGQGTKVEIK

SEQ ID NO: 163 - protein, 106 aa, synthetic construct
  REGION 1..106: note = PR003266 VL
DIQMTQSPSTLSASVGDRVTITCRASQSISSWLAWYQQKPGKAPKLLIYKASSLESGVPSRFSGSGSGTEFTLTISSLQPDDFATYYCQQYNSYSTFGQGTKLEIK

SEQ ID NO: 164 - protein, 107 aa, synthetic construct
  REGION 1..107: note = PR004480 VL, PR004483 VL, PR004485 VL
DIQMTQSPSTLSASVGDRVTITCRASQSISSWLAWYQQKPGKAPKLLIYKASSLESGVPSRFSGSGSGTEFTLTISSLQPDDLATYYCQQYQSYSYTFGQGTKLEIK

SEQ ID NO: 165 - protein, 107 aa, synthetic construct
  REGION 1..107: note = PR004481 VL, PR004484 VL, PR004486 VL
DIQMTQSPSTLSASVGDRVTITCRASQSISSWLAWYQQKPGKAPKLLIYKASSLESGVPSRFSGSGSGTEFTLTISSLQPDDLATYYCQQYNAYSYTFGQGTKLEIK

SEQ ID NO: 166 - protein, 107 aa, synthetic construct
  REGION 1..107: note = PR004488 VL, PR004491 VL, PR004493 VL
DIQMTQSPSSLSASVGDRVTITCRASQGISNYLAWYQQKPGKVPKLLIYAASTLQSGVPSRFSGSGSGTDFTLTISSLQPEDVATYYCQKYQSAPFTFGPGTKVDIK

SEQ ID NO: 167 - protein, 107 aa, synthetic construct
  REGION 1..107: note = PR004489 VL, PR004492 VL, PR004494 VL
DIQMTQSPSSLSASVGDRVTITCRASQGISNYLAWYQQKPGKVPKLLIYAASTLQSGVPSRFSGSGSGTDFTLTISSLQPEDVATYYCQKYNAAPFTFGPGTKVDIK

SEQ ID NO: 168 - protein, 444 aa, synthetic construct
  REGION 1..444: note = PR003181 HC
QVQLQQSGAELVKPGASVKLSCKASGYTFTSHDINWVRQRPEQGLEWIGWIFPGDGSTKFNEKFKGKATLTTDKSSSTAYIQLSRLTSEDSAVYFCARNSFYSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 169 - protein, 451 aa, synthetic construct
  REGION 1..451: note = PR003248 HC
QVQLVESGGGVVQPGRSLRLSCAASGFTFSDYGLHWVRQAPGRGLEWVAVIWYDGSEEYYADSVKGRFTISRDNSKKSLYLQMNSLRAEDTAVYFCARDRNSGALGYFPHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 451 aa, synthetic construct
  REGION 1..451: note = PR003249 HC
QVQLVESGGGVVQPGRSLRLSCAASGFTFSDYGIHWVRQAPGKGLEWVAVIWYDGSDKYYADSVRGRFTISRDNSKNTLSLQMNSLRVEDTAVYYCARDRSSGAMGYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 451 aa, synthetic construct
  REGION 1..451: note = PR003250 HC
QVQLVESGGGVVQPGRSLRLSCAASGFTFSDYGIHWVRQAPGKGLEWVAVIWYDGSDKYYADSVRGRFTISRDNSKNTLSLQMNSLRAEDTAVYYCARDRSSGAMGYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 450 aa, synthetic construct
  REGION 1..450: note = PR003251 HC
QVQLVESGGGVVQPGRSLRLSCAVSGFTFSRYGMHWVRQAPGKGLDWVAVIWYNGNNKYYTDSVKGRFTISRDNSKNTLFLQMNSLRAEDTAVYYCARDGAYSYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 450 aa, synthetic construct
  REGION 1..450: note = PR003252 HC
QVQLVESGGGVVQPGRSLRLSCAASGFTFSNYAMHWVRQVPGKGLEWVALIWYDGSEKYYADSVKGRFTISRDDSKNTLFLQMSSLRAEDTAVYSCVREYSSGWGYFDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 450 aa, synthetic construct
  REGION 1..450: note = PR003254 HC
QVQLVESGGGVVQPGRSLRLSCAASGFTFSSYGMHWVRQAPGKGLEWVADIWYNGNNKYYADSVKGRFTISRDNSKNTLYLQMNSLRAEDTALYYCARDGGYSYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 450 aa, synthetic construct
  REGION 1..450: note = PR003259 HC
QVQLVESGGGVVQPGRSLRLSCAASGFTFSNYGMHWVRQVPGKGLEWVAVIWYDGSEKDYADSVKGRFTISRDNSKNTLYLQMNSLRVEDTAVYYCAKDLISGIYYVDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 450 aa, synthetic construct
  REGION 1..450: note = PR003260 HC
QVQLVESGGGVVQPGRSLRLPCAASGFTFSRYGMHWVRQAPGKGLEWVADIWYDGSKKYYADSVKGRFTISRDNSKNTLYLQMNSLRAEDTAVYFCTRDGGYSYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 450 aa, synthetic construct
  REGION 1..450: note = PR003261 HC
QVQLVESGGGVVQPGRSLRLSCAASGFTFSNYGMHWVRQAPGKGLEWVAVIWYDGTNKNYADSVKGRFTISRDNSKNTLDLQMNSLRAEDTAVYFCARDRNTAMDYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 450 aa, synthetic construct
  REGION 1..450: note = PR003262 HC
QVQLVESGGGVVQPGRSLRLSCAASGFTFSRYGIHWVRQAPGKGLEWVAVIWYNGRNKYYVDSVKGRFTISRDNSKNTLYLQMNSLRAEDTAVYYCARDGGYSYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 453 aa, synthetic construct
  REGION 1..453: note = PR003263 HC
QVQLVESGGGVVQPGRSLRLSCAASGFTFSYYGLNWVRQAPGKGLEWVAVIWFDGTNKYYADSVKGRFTISRDDSKNTLYLQMNSLRAEDTAVYYCARGYGSGSYYNYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 451 aa, synthetic construct
  REGION 1..451: note = PR003264 HC
QVQLVQSGAEVKKPGSSVKVSCKTSGGTFSSYAISWVRQAPGQGLEWMGGIIPLIGTANYAQNFQGRVTITADESTRTAYMELSSLRSDDTAVYYCARDTMVRGVRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 451 aa, synthetic construct
  REGION 1..451: note = PR003265 HC
QVQLVESGGGVVQPGRSLRLSCAASGFTFSTYGLHWVRQAPGKGLEWVAVIWYDESNEYYADSVKGRFTISRDNSKNTLYLQMNSLRAEDTALYYCARLAHDYGGTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 451 aa, synthetic construct
  REGION 1..451: note = PR003266 HC
QVQLVESGGGVVQPGRSLRLSCAASGFTFSDYGMHWVRQAPGKGLEWVAIIWYDGSNKYYADSVKGRFTISRDNSKNTLYLQMNSLRAEDTAVYYCARDRSSGALGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 447 aa, synthetic construct
  REGION 1..447: note = PR004260 HC
QVQLQQSGAELVKPGASVKLSCKASGYTFTSHDINWVRQRPEQGLEWIGWIFPGDGSTKFNEKFKGKATLTTDKSSSTAYIQLSRLTSEDSAVYFCARNSFYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 451 aa, synthetic construct
  REGION 1..451: note = PR004261 HC, PR004485 HC, PR004486 HC
QVQLVESGGGVVQPGRSLRLSCAASGFTFSDYGIHWVRQAPGKGLEWVAVIWYDGSDKYYADSVRGRFTISRDNSKNTLSLQMNSLRVEDTAVYYCARDRSSGAMGYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 450 aa, synthetic construct
  REGION 1..450: note = PR004262 HC, PR004493 HC, PR004494 HC
QVQLVESGGGVVQPGRSLRLSCAASGFTFSNYGMHWVRQVPGKGLEWVAVIWYDGSEKDYADSVKGRFTISRDNSKNTLYLQMNSLRVEDTAVYYCAKDLISGIYYVDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 451 aa, synthetic construct
  REGION 1..451: note = PR004263 HC
QVQLVQSGAEVKKPGSSVKVSCKTSGGTFSSYAISWVRQAPGQGLEWMGGIIPLIGTANYAQNFQGRVTITADESTRTAYMELSSLRSDDTAVYYCARDTMVRGVRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 451 aa, synthetic construct
  REGION 1..451: note = PR004479 HC, PR004480 HC, PR004481 HC
QVQLVESGGGVVQPGRSLRLSCAASGFTFSDYGIHWVRQAPGKGLEWVAVIWYEGSDKYYADSVRGRFTISRDNSKNTLSLQMNSLRVEDTAVYYCARDRSSGAMGYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 451 aa, synthetic construct
  REGION 1..451: note = PR004482 HC, PR004483 HC, PR004484 HC
QVQLVESGGGVVQPGRSLRLSCAASGFTFSDYGIHWVRQAPGKGLEWVAVIWYDASDKYYADSVRGRFTISRDNSKNTLSLQMNSLRVEDTAVYYCARDRSSGAMGYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 450 aa, synthetic construct
  REGION 1..450: note = PR004487 HC, PR004488 HC, PR004489 HC
QVQLVESGGGVVQPGRSLRLSCAASGFTFSNYGMHWVRQVPGKGLEWVAVIWYEGSEKDYADSVKGRFTISRDNSKNTLYLQMNSLRVEDTAVYYCAKDLISGIYYVDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 450 aa, synthetic construct
  REGION 1..450: note = PR004490 HC, PR004491 HC, PR004492 HC
QVQLVESGGGVVQPGRSLRLSCAASGFTFSNYGMHWVRQVPGKGLEWVAVIWYDASEKDYADSVKGRFTISRDNSKNTLYLQMNSLRVEDTAVYYCAKDLISGIYYVDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 219 aa, synthetic construct
  REGION 1..219: note = PR003181 LC, PR004260 LC
DIVMSQSPSSLAVSAGEKVTMSCKSSQSLLNSRTRKNQLAWYQQKPGQSPKLLIYWASTRESGVPDRFTGSGSGTDFTLTISSVQAEDLAVYYCKQSYNLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 213 aa, synthetic construct
  REGION 1..213: note = PR003248 LC
DIQMTQSPSTLSASVGDRVTITCRASQSINNWLAWYQQKPGKAPKLLIYKASSLESGVPSRFSGSGSGTEFTLTISSLQPDDFATYYCQQYNSYSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 214 aa, synthetic construct
  REGION 1..214: note = PR003249 LC, PR003250 LC, PR004261 LC, PR004479 LC, PR004482 LC
DIQMTQSPSTLSASVGDRVTITCRASQSISSWLAWYQQKPGKAPKLLIYKASSLESGVPSRFSGSGSGTEFTLTISSLQPDDLATYYCQQYNSYSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 214 aa, synthetic construct
  REGION 1..214: note = PR003251 LC
EIVMTQSPATLSVSPGEGATLSCRASQSVSSNLAWYQQKPGQAPRLLIYGASTRATGIPARFSGSGSGTEFTLTISSLQSEDFAVYYCQQYNIWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 213 aa, synthetic construct
  REGION 1..213: note = PR003252 LC
DIQMTQSPSTLSASVGDRVTITCRASQSISSWLAWYQQKPGKAPKLLIYKASSLKSGVPSRFSGSGSGTEFTLTISSLQPDDFATYYCLQYNSYYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 214 aa, synthetic construct
  REGION 1..214: note = PR003254 LC
EIVMTQSPATLSVSPGERATLSCRASQSVSSYLAWYQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 214 aa, synthetic construct
  REGION 1..214: note = PR003259 LC, PR004262 LC, PR004487 LC, PR004490 LC
DIQMTQSPSSLSASVGDRVTITCRASQGISNYLAWYQQKPGKVPKLLIYAASTLQSGVPSRFSGSGSGTDFTLTISSLQPEDVATYYCQKYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 214 aa, synthetic construct
  REGION 1..214: note = PR003260 LC
EIVMTQSPATLSVSPGERATLSCRASQSVSSNLAWYQQKPGQAPRLLIYYASTRAPGIPARFSGSESGTEFTLTISSLQSEDFAVYYCQQYNIWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 214 aa, synthetic construct
  REGION 1..214: note = PR003261 LC
DIQMTQSPSTLSASVGDRVTITCRASQSISSWLAWYQQKPGKAPKLLIYKASSLESGVPSRFSGSGSGTEFTLTISSLQPDDFASYYCQQYNSYSVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 214 aa, synthetic construct
  REGION 1..214: note = PR003262 LC
EIVMTQSPATLSVSPGERATLSCRASQSFSNNLAWYQQKPGQAPRLLIYGASTRATGIPARFSGSGSGTEFTLTISSLQSEDFAVYYCHQYNIWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 214 aa, synthetic construct
  REGION 1..214: note = PR003263 LC
EIVMTQSPATLSVSPGERATLSCRASQSVSSNLAWYQQKPGQAPRLLIYGASTGATGIPARFSGSGSGTDFTLTISSLQSEDFAVYYCQQYYKWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 214 aa, synthetic construct
  REGION 1..214: note = PR003264 LC, PR004263 LC
DIQLTQSPSFLSASVGDRVTITCRASQGISSYFAWYQQKPGKAPKLLIYAASTLQSGVPSRFSGSGSGTEFTLTISSLQPEDFATYYCQHLNRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 215 aa, synthetic construct
  REGION 1..215: note = PR003265 LC
EIVMTQSPAILSVSPGERATLSCRASQSISYNLAWYQQKPGQAPRLLIYGASIRATGIPARFSGSGSGTDFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 213 aa, synthetic construct
  REGION 1..213: note = PR003266 LC
DIQMTQSPSTLSASVGDRVTITCRASQSISSWLAWYQQKPGKAPKLLIYKASSLESGVPSRFSGSGSGTEFTLTISSLQPDDFATYYCQQYNSYSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 214 aa, synthetic construct
  REGION 1..214: note = PR004480 LC, PR004483 LC, PR004485 LC
DIQMTQSPSTLSASVGDRVTITCRASQSISSWLAWYQQKPGKAPKLLIYKASSLESGVPSRFSGSGSGTEFTLTISSLQPDDLATYYCQQYQSYSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, synthetic construct
  REGION 1..214: note = PR004481 LC, PR004484 LC, PR004486 LC
DIQMTQSPSTLSASVGDRVTITCRASQSISSWLAWYQQKPGKAPKLLIYKASSLESGVPSRFSGSGSGTEFTLTISSLQPDDLATYYCQQYNAYSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 214 aa, synthetic construct
  REGION 1..214: note = PR004488 LC, PR004491 LC, PR004493 LC
DIQMTQSPSSLSASVGDRVTITCRASQGISNYLAWYQQKPGKVPKLLIYAASTLQSGVPSRFSGSGSGTDFTLTISSLQPEDVATYYCQKYQSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - protein, 214 aa, synthetic construct
  REGION 1..214: note = PR004489 LC, PR004492 LC, PR004494 LC
DIQMTQSPSSLSASVGDRVTITCRASQGISNYLAWYQQKPGKVPKLLIYAASTLQSGVPSRFSGSGSGTDFTLTISSLQPEDVATYYCQKYNAAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0136696 A1
(43) Pub. Date: May 1, 2025

(54) ANTI-B7-H7 ANTIBODY OR ANTIGEN-BINDING FRAGMENT THEREOF, AND PREPARATION METHOD THEREFOR AND USE THEREOF

(71) Applicant: Harbour BioMed (Shanghai) Co., Ltd., Shanghai (CN)

(72) Inventors: Xiaodong Wu, Shanghai (CN); Yongqiang Wang, Shanghai (CN); Xiaocheng Lv, Shanghai (CN); Fei Chen, Shanghai (CN); Xuemei Li, Shanghai (CN); Jiuqiao Zhao, Shanghai (CN); Yiping Rong, Shanghai (CN)

(21) Appl. No.: 18/730,431

(22) PCT Filed: Jan. 17, 2023
(86) PCT No.: PCT/CN2023/072622

§ 371 Date: Jul. 19, 2024

(30) Foreign Application Priority Data
Jan. 21, 2022 (CN) 202210071861.3

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/33; C07K 2317/76; C07K 2317/92; C07K 2317/94

(57) ABSTRACT

Disclosed are an anti-B7-H7 antibody or an antigen-binding fragment thereof, a preparation method therefor and the use thereof. The anti-B7-H7 antibody or antigen-binding fragment thereof of the present invention has high affinity and high biological activity for B7-H7, and can effectively block the binding of B7-H7 to a receptor CD28H thereof and the binding of B7-H7 to a receptor KIR3DL3 thereof; furthermore, the anti-B7-H7 antibody or antigen-binding fragment thereof only specifically binds to B7-H7 without cross-reacting with other member proteins of the B7 family, and has good stability; in addition, the anti-B7-H7 antibody or antigen-binding fragment thereof shows strong in vivo anti-tumor activity.

### CROSS-REFERENCE TO RELATED APPLICATIONS

The present patent application is a U.S. National Stage Application, filed under 35 U.S.C. § 371, of International Application No. PCT/CN2023/072622, filed Jan. 17, 2023, which claims the right of priority for the Chinese patent application 202210071861.3 filed on Jan. 21, 2022, the content of which is incorporated herein by reference in their entireties.

### REFERENCE TO SEQUENCE LISTING

This application is being filed electronically and includes an electronically submitted sequence listing in.xml format. The.xml file contains a sequence listing entitled “WO2023138579_Sequence_Listing.xml” created on Jul. 27, 2023, and having a size of 282,109 bytes. The sequence listing contained in this.xml file is part of the specification and is incorporated herein by reference in its entirety.

### TECHNICAL FIELD

The present invention relates to the field of biomedicine, in particular to an anti-B7-H7 antibody or an antigen-binding fragment thereof, a preparation method therefor and the use thereof.

### BACKGROUND ART

Immune checkpoint molecules are often highly expressed in the tumor microenvironment, which promote tumors to evade the attack from the immune system by inhibiting T cell activation and inducing T cell exhaustion. Inhibition of Immune checkpoints is the most effective and cutting edge treatment among current anti-tumor immunotherapies. For example, immune checkpoint inhibitors against PD1, PD-L1 and CTLA-4 have shown strong anti-tumor activity clinically. Thus the 2018 Nobel Prize in Physiology or Medicine was awarded to two scientists who discovered the negative immune regulation of PD1 and CTLA-4 and made outstanding contributions to the treatment of cancers.

Although treatment with anti-PD1, PD-L1 and CTLA-4 has achieved good efficacy clinically, the response rate of patients is not high, with most of the patients being resistant to the treatment of these targets, or the efficacy is not obvious. It has been proved that the tumor microenvironment is a very complex comprehensive mechanism. A variety of immunosuppressive molecules, suppressor cells and special tumor tissue structures together block the occurrence of normal immune responses. It is conceivable that a single blocking of a certain immune checkpoint is often difficult to achieve good efficacy. In addition to these targets PD1, PD-L1 and CTLA-4, other immune checkpoints such as Tim3, Lag3, TIGIT and other molecules are also highly expressed in the tumor microenvironment. Current studies on these immune checkpoints have shown that inhibition of these targets, or same in combination with anti-PD1/PD-L1 antibodies, can further enhance the anti-tumor efficacy.

The B7 family is an important family of immune system co-stimulatory/inhibitory molecules. There are currently 10 molecules in the B7 family, namely CD80 (B7.1), CD86 (B7.2), B7H1 (PD-L1/CD274), B7-DC (PD-L2/CD273), B7H2 (ICOSL), B7H3 (CD276), B7H4 (B7S1/B7x/Vtcn1), B7H5 (VISTA), B7H6 and B7-H7 (HHLA2). Multiple members of the B7 family or their receptors have been shown to be immune checkpoints, such as PD-L1/PD1, CTLA4, B7H4 and VISTA.

B7-H7 is currently one of the latest B7 family molecules, and unlike other molecules of this family, B7-H7 has 3 extracellular Ig domains. The in-vitro function of B7-H7 is somewhat controversial and may be related to the different receptors on which it acts. One of these receptors is CD28H, and it has been reported that B7-H7 acts on CD28H to promote the function of T cells or NK cells (Nat Commun. 2013; 4:2043; Cancer Immunol Res. 2019; 7 (6): 939-951.). Additionally, it has been reported that B7-H7 can act on KIR3DL3, thereby inhibiting the function of T cells (Cancer Immunol Res. 2020 Nov. 23; canimm.0315.2020.).

In normal tissues, B7-H7 is mainly highly expressed in tissues such as the gastrointestinal tract and bile duct. Studies of patient samples have found that the B7-H7 molecule is highly expressed in many tumor types, such as breast cancer, lung cancer, bowel cancer and pancreatic cancer, wherein its expression abundance is often negatively correlated with prognosis and survival (Clin Cancer Res. 2015 May 15; 21 (10): 2359-2366; Onko Targets Ther. 2018; 11:1563-1570). Therefore, B7-H7 acts as an immune checkpoint in vivo, inhibiting immune responses. Blocking the action of B7-H7 by preparing neutralizing antibodies to reactivate the immune system is a promising means to treat tumors with high expression of B7-H7.

At present, there are few studies on B7-H7, or all of them are under preclinical development. Therefore, there is an urgent need for the development of human anti-B7-H7 antibodies having high affinity and high biological activity and showing strong in vivo anti-tumor activity.

### SUMMARY OF THE INVENTION

The technical problem to be solved by the present invention is to overcome the defect of lacking anti-B7-H7 antibodies in the prior art, and an anti-B7-H7 antibody or an antigen-binding fragment thereof, a preparation method therefor and the use thereof are provided.

In order to solve the above-mentioned technical problem, a first aspect of the present invention provides an anti-B7-H7 antibody or an antigen-binding fragment thereof comprising a light chain variable region (VL) and/or a heavy chain variable region (VH); wherein the VL comprises complementarity determining regions (CDRs) as follows or mutations thereof: LCDR1 set forth in an amino acid sequence of SEQ ID NO: 82, LCDR2 set forth in any one of amino acid sequences of SEQ ID NOs: 94-100, and/or LCDR3 set forth in any one of amino acid sequences of SEQ ID NOs: 111, 112, 114-116, 120 and 121;
the VH comprises CDRs as follows or mutations thereof: HCDR1 set forth in an amino acid sequence of SEQ ID NO: 7, HCDR2 set forth in an amino acid sequence of SEQ ID NO: 34 or 36, and/or HCDR3 set forth in any one of amino acid sequences of SEQ ID NOs: 57-68;
wherein the mutation is 3, 2 or 1 amino acid insertion, deletion or substitution in the amino acid sequence of the CDR.

In the present application, in the sentence similar to the “with 3, 2 or 1 amino acid insertion, deletion or substitution”, the “amino acid mutation” refers to amino acid mutations in the sequence of a variant comparing to an original amino acid sequence, including amino acid insertion, deletion or substitutions occurring on the basis of the original amino acid sequence. An exemplary explanation is that the mutation of CDRs can comprise 3, 2 or 1 amino acid mutation, and the same or a different number of amino acid residues can optionally be selected for mutation between these CDRs, for example, it can be 1 amino acid mutation for CDR1, but no amino acid mutation for CDR2 and CDR3.

In the present application, the mutations may include mutations as currently well known to those skilled in the art, such as some mutations that may be performed on antibodies during the production or use of the antibodies, such as mutations at potential sites, in particular the sites of post-translational modifications (PTMs) in the CDR region, including mutations at sites such as aggregation sites of antibodies, asparagine deamidation sensitive sites (NG, NS, NH, etc.), aspartate isomerization sensitive sites (DG, DP), N-glycosylation sensitive sites (N-{P}S/T), and oxidation sensitive sites.

Preferably, the mutation in LCDR1 is 3, 2 or 1 amino acid substitution of S5G, 16V/F, S7N, S8N/Y, W9N/Y and L10F in the amino acid sequence set forth in SEQ ID NO: 82; the amino acid sequence having the mutation in the LCDR1 is preferably set forth in any one of SEQ ID NOs: 81 and 83-88.

Preferably, the mutation in LCDR3 is 1 amino acid substitution of N4Q, S5A and Y8V in the amino acid sequence set forth in SEQ ID NO: 112, or the mutation in LCDR3 is 2 or 1 amino acid substitution of Q1H, N4Y, N51/K and L8Y in the amino acid sequence set forth in SEQ ID NO: 115, or the mutation in LCDR3 is 1 amino acid substitution of N4Q and S5A in the amino acid sequence set forth in SEQ ID NO: 116; the amino acid sequence having the mutation in the LCDR3 is preferably set forth in any one of SEQ ID NOs: 113, 117-119 and 122-125.

Preferably, the mutation in HCDR1 is 2 or 1 amino acid substitution of F2G and D6R/N/S/Y/T in the amino acid sequence set forth in SEQ ID NO: 7; the amino acid sequence having the mutation in the HCDR1 is preferably set forth in any one of SEQ ID NOs: 8-13.

Preferably, the mutation in HCDR2 is 2 or 1 amino acid substitution of Y2F, D3N/E, G4E/A, S5N/T/R and N6E/D/K in the amino acid sequence set forth in SEQ ID NO: 36; the amino acid sequence having the mutation in the HCDR2 is preferably set forth in any one of SEQ ID NOs: 27-33, 35 and 37-40.

The S5G as described above generally refers to a mutation of amino acid S to G at position 5 of the amino acid sequence set forth in SEQ ID NO: 82, and other amino acid substitutions such as 16V/F, S7N, S8N/Y, W9N/Y and L10F, and so forth, the meaning of which should be understood by those skilled in the art.

Preferably, the LCDR1 comprises an amino acid sequence set forth in SEQ ID NO: 87, the LCDR2 comprises an amino acid sequence set forth in SEQ ID NO: 97, and the LCDR3 comprises an amino acid sequence set forth in SEQ ID NO: 120; or the LCDR1 comprises an amino acid sequence set forth in SEQ ID NO: 82, the LCDR2 comprises an amino acid sequence set forth in SEQ ID NO: 94, and the LCDR3 comprises an amino acid sequence set forth in SEQ ID NO: 112; or the LCDR1 comprises an amino acid sequence set forth in SEQ ID NO: 82, the LCDR2 comprises an amino acid sequence set forth in SEQ ID NO: 94, and the LCDR3 comprises an amino acid sequence set forth in SEQ ID NO: 122; or the LCDR1 comprises an amino acid sequence set forth in SEQ ID NO: 82, the LCDR2 comprises an amino acid sequence set forth in SEQ ID NO: 94, and the LCDR3 comprises an amino acid sequence set forth in SEQ ID NO: 123; or the LCDR1 comprises an amino acid sequence set forth in SEQ ID NO: 85, the LCDR2 comprises an amino acid sequence set forth in SEQ ID NO: 97, and the LCDR3 comprises an amino acid sequence set forth in SEQ ID NO: 116; or the LCDR1 comprises an amino acid sequence set forth in SEQ ID NO: 85, the LCDR2 comprises an amino acid sequence set forth in SEQ ID NO: 97, and the LCDR3 comprises an amino acid sequence set forth in SEQ ID NO: 124; or the LCDR1 comprises an amino acid sequence set forth in SEQ ID NO: 85, the LCDR2 comprises an amino acid sequence set forth in SEQ ID NO: 97, and the LCDR3 comprises an amino acid sequence set forth in SEQ ID NO: 125; or the LCDR 1 comprises an amino acid sequence set forth in SEQ ID NO: 81, the LCDR2 comprises an amino acid sequence set forth in SEQ ID NO: 94, and the LCDR3 comprises an amino acid sequence set forth in SEQ ID NO: 111; or the LCDR1 comprises an amino acid sequence set forth in SEQ ID NO: 82, the LCDR2 comprises an amino acid sequence set forth in SEQ ID NO: 94, and the LCDR3 comprises an amino acid sequence set forth in SEQ ID NO: 111; or the LCDR1 comprises an amino acid sequence set forth in SEQ ID NO: 82, the LCDR2 comprises an amino acid sequence set forth in SEQ ID NO: 94, and the LCDR3 comprises an amino acid sequence set forth in SEQ ID NO: 117; or the LCDR1 comprises an amino acid sequence set forth in SEQ ID NO: 82, the LCDR2 comprises an amino acid sequence set forth in SEQ ID NO: 96, and the LCDR3 comprises an amino acid sequence set forth in SEQ ID NO: 114; or the LCDR1 comprises an amino acid sequence set forth in SEQ ID NO: 83, the LCDR2 comprises an amino acid sequence set forth in SEQ ID NO: 95, and the LCDR3 comprises an amino acid sequence set forth in SEQ ID NO: 113; or the LCDR1 comprises an amino acid sequence set forth in SEQ ID NO: 83, the LCDR2 comprises an amino acid sequence set forth in SEQ ID NO: 98, and the LCDR3 comprises an amino acid sequence set forth in SEQ ID NO: 113; or the LCDR1 comprises an amino acid sequence set forth in SEQ ID NO: 83, the LCDR2 comprises an amino acid sequence set forth in SEQ ID NO: 99, and the LCDR3 comprises an amino acid sequence set forth in SEQ ID NO: 119; or the LCDR1 comprises an amino acid sequence set forth in SEQ ID NO: 84, the LCDR2 comprises an amino acid sequence set forth in SEQ ID NO: 95, and the LCDR3 comprises an amino acid sequence set forth in SEQ ID NO: 115; or the LCDR1 comprises an amino acid sequence set forth in SEQ ID NO: 86, the LCDR2 comprises an amino acid sequence set forth in SEQ ID NO: 95, and the LCDR3 comprises an amino acid sequence set forth in SEQ ID NO: 118; or the LCDR1 comprises an amino acid sequence set forth in SEQ ID NO: 88, the LCDR2 comprises an amino acid sequence set forth in SEQ ID NO: 100, and the LCDR3 comprises an amino acid sequence set forth in SEQ ID NO: 121.

Preferably, the HCDR1 comprises an amino acid sequence set forth in SEQ ID NO: 12, the HCDR2 comprises an amino acid sequence set forth in SEQ ID NO: 34, and the HCDR3 comprises an amino acid sequence set forth in SEQ ID NO: 66; or the HCDR1 comprises an amino acid sequence set forth in SEQ ID NO: 7, the HCDR2 comprises an amino acid sequence set forth in SEQ ID NO: 28, and the HCDR3 comprises an amino acid sequence set forth in SEQ ID NO: 58; or the HCDR1 comprises an amino acid sequence set forth in SEQ ID NO: 7, the HCDR2 comprises an amino acid sequence set forth in SEQ ID NO: 37, and the HCDR3 comprises an amino acid sequence set forth in SEQ ID NO: 58; or the HCDR1 comprises an amino acid sequence set forth in SEQ ID NO: 7, the HCDR2 comprises an amino acid sequence set forth in SEQ ID NO: 38, and the HCDR3 comprises an amino acid sequence set forth in SEQ ID NO: 58; or the HCDR1 comprises an amino acid sequence set forth in SEQ ID NO: 9, the HCDR2 comprises an amino acid sequence set forth in SEQ ID NO: 27, and the HCDR3 comprises an amino acid sequence set forth in SEQ ID NO: 63; or the HCDR1 comprises an amino acid sequence set forth in SEQ ID NO: 9, the HCDR2 comprises an amino acid sequence set forth in SEQ ID NO: 39, and the HCDR3 comprises an amino acid sequence set forth in SEQ ID NO: 63; or the HCDR1 comprises an amino acid sequence set forth in SEQ ID NO: 9, the HCDR2 comprises an amino acid sequence set forth in SEQ ID NO: 40, and the HCDR3 comprises an amino acid sequence set forth in SEQ ID NO: 63; or the HCDR1 comprises an amino acid sequence set forth in SEQ ID NO: 7, the HCDR2 comprises an amino acid sequence set forth in SEQ ID NO: 27, and the HCDR3 comprises an amino acid sequence set forth in SEQ ID NO: 57; or the HCDR1 comprises an amino acid sequence set forth in SEQ ID NO: 7, the HCDR2 comprises an amino acid sequence set forth in SEQ ID NO: 28, and the HCDR3 comprises an amino acid sequence set forth in SEQ ID NO: 59; or the HCDR1 comprises an amino acid sequence set forth in SEQ ID NO: 7, the HCDR2 comprises an amino acid sequence set forth in SEQ ID NO: 36, and the HCDR3 comprises an amino acid sequence set forth in SEQ ID NO: 68; or the HCDR1 comprises an amino acid sequence set forth in SEQ ID NO: 8, the HCDR2 comprises an amino acid sequence set forth in SEQ ID NO: 29, and the HCDR3 comprises an amino acid sequence set forth in SEQ ID NO: 60; or the HCDR1 comprises an amino acid sequence set forth in SEQ ID NO: 8, the HCDR2 comprises an amino acid sequence set forth in SEQ ID NO: 30, and the HCDR3 comprises an amino acid sequence set forth in SEQ ID NO: 62; or the HCDR1 comprises an amino acid sequence set forth in SEQ ID NO: 8, the HCDR2 comprises an amino acid sequence set forth in SEQ ID NO: 32, and the HCDR3 comprises an amino acid sequence set forth in SEQ ID NO: 62; or the HCDR1 comprises an amino acid sequence set forth in SEQ ID NO: 9, the HCDR2 comprises an amino acid sequence set forth in SEQ ID NO: 27, and the HCDR3 comprises an amino acid sequence set forth in SEQ ID NO: 61; or the HCDR1 comprises an amino acid sequence set forth in SEQ ID NO: 9, the HCDR2 comprises an amino acid sequence set forth in SEQ ID NO: 31, and the HCDR3 comprises an amino acid sequence set forth in SEQ ID NO: 64; or the HCDR1 comprises an amino acid sequence set forth in SEQ ID NO: 10, the HCDR2 comprises an amino acid sequence set forth in SEQ ID NO: 29, and the HCDR3 comprises an amino acid sequence set forth in SEQ ID NO: 62; or the HCDR1 comprises an amino acid sequence set forth in SEQ ID NO: 11, the HCDR2 comprises an amino acid sequence set forth in SEQ ID NO: 33, and the HCDR3 comprises an amino acid sequence set forth in SEQ ID NO: 65; or the HCDR1 comprises an amino acid sequence set forth in SEQ ID NO: 13, the HCDR2 comprises an amino acid sequence set forth in SEQ ID NO: 35, and the HCDR3 comprises an amino acid sequence set forth in SEQ ID NO: 67.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 87, 97 and 120, respectively, and the VH comprises HCDR1, HCDR2 and HCDR3 having amino acid sequences set forth in SEQ ID NOs: 12, 34 and 66, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 28 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 37 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 37 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 37 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 38 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 38 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 38 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 28 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 28 and 58, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 27 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 39 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 39 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 39 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 40 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 40 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 40 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 27 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 27 and 63, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 81, 94 and 111, respectively, and the VH comprises HCDR1, HCDR2 and HCDR3 having amino acid sequences set forth in SEQ ID NOs: 7, 27 and 57, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 28 and 59, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 83, 95 and 113, respectively, and the VH comprises HCDR1, HCDR2 and HCDR3 having amino acid sequences set forth in SEQ ID NOs: 8, 29 and 60, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 96 and 114, respectively, and the VH comprises HCDR1, HCDR2 and HCDR3 having amino acid sequences set forth in SEQ ID NOs: 9, 27 and 61, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 84, 95 and 115, respectively, and the VH comprises HCDR1, HCDR2 and HCDR3 having amino acid sequences set forth in SEQ ID NOs: 10, 29 and 62, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 83, 98 and 113, respectively, and the VH comprises HCDR1, HCDR2 and HCDR3 having amino acid sequences set forth in SEQ ID NOs: 8, 30 and 62, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 117, respectively, and the VH comprises HCDR1, HCDR2 and HCDR3 having amino acid sequences set forth in SEQ ID NOs: 9, 31 and 64, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 86, 95 and 118, respectively, and the VH comprises HCDR1, HCDR2 and HCDR3 having amino acid sequences set forth in SEQ ID NOs: 8, 32 and 62, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 83, 99 and 119, respectively, and the VH comprises HCDR1, HCDR2 and HCDR3 having amino acid sequences set forth in SEQ ID NOs: 11, 33 and 65, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 88, 100 and 121, respectively, and the VH comprises HCDR1, HCDR2 and HCDR3 having amino acid sequences set forth in SEQ ID NOs: 13, 35 and 67, respectively.

In a certain preferred embodiment, in the anti-B7-H7 antibody or the antigen-binding fragment thereof, the VL comprises LCDR1, LCDR2 and LCDR3 having amino acid sequences set forth in SEQ ID NOs: 82, 94 and 111, respectively, and the VH comprises HCDR1, HCDR2 and HCDR3 having amino acid sequences set forth in SEQ ID NOs: 7, 36 and 68, respectively.

In the present invention, the amino acid sequences of the CDRs listed above are shown according to the rules of Chothia definition. However, it is well known to those skilled in the art that the CDRs of an antibody can be defined in the art by a variety of methods, such as the rules of Kabat definition based on sequence variability (see, Kabat et al., Sequences of Proteins of Immunological Interest, fifth edition, National Institutes of Health, Bethesda, Maryland (1991)), and the rules of Chothia definition based on the location of a structural loop region (see J Mol Biol 273:927-48, 1997). In the technical solutions of the present invention, amino acid residues in variable domain sequences can also be determined according to the rules of Combined definition that incorporates both Kabat definition and Chothia definition. The rules of Combined definition refers to the combination of the ranges of Kabat definition and Chothia definition, based on which a larger scope is taken, see Table 1-6 in the examples for details. It should be understood by those skilled in the art that unless otherwise specified, the terms “CDR” and “complementarity determining region” of a given antibody or region thereof (for example, a variable region) should be understood to encompass the complementarity determining region as defined by any of the above-mentioned known schemes as described in the present invention. Although the scope of protection as claimed in the present invention is based on the sequences shown according to the rules of Chothia definition, corresponding amino acid sequences according to the rules of other CDR definitions shall also fall within the scope of protection of the present invention.

Preferably, the light chain variable region (VL) further comprises a light chain variable region framework region (VL FWR), the VL FWR is a light chain variable region framework region of a human antibody, and the gene encoding the VL FWR is preferably derived from a germline V gene IGKV1-5*03, IGKV3-15*01, IGKV1-27*01 or IGKV1-9*01. More preferably, the VL FWR comprises VL FWR1 having an amino acid sequence set forth in any one of sequences of SEQ ID NOs: 74-79 or mutations thereof, VL FWR2 having an amino acid sequence set forth in any one of sequences of SEQ ID NOs: 90-92 or mutations thereof, VL FWR3 having an amino acid sequence set forth in any one of sequences of SEQ ID NOs: 102-109 or mutations thereof, and VL FWR4 having an amino acid sequence set forth in any one of sequences of SEQ ID NOs: 127-130 or mutations thereof. The mutation is 3, 2 or 1 amino acid insertion, deletion, substitution or duplication in the amino acid sequence of the VL FWR.

Preferably, the heavy chain variable region (VH) further comprises a heavy chain variable region framework region (VH FWR), the VH FWR is a heavy chain variable region framework region of a human antibody, and the gene encoding the VH FWR is preferably derived from a germline V gene IGHV3-33*01, IGHV3-33*06 or IGHV1-69*01. More preferably, the VH FWR comprises VH FWR1 having an amino acid sequence set forth in any one of sequences of SEQ ID NOs: 2-5 or mutations thereof, VH FWR2 having an amino acid sequence set forth in any one of sequences of SEQ ID NOs: 15-25 or mutations thereof, VH FWR3 having an amino acid sequence set forth in any one of sequences of SEQ ID NOs: 42-55 or mutations thereof, and VH FWR4 having an amino acid sequence set forth in any one of sequences of SEQ ID NOs: 70-72 or mutations thereof. The mutation is 3, 2 or 1 amino acid insertion, deletion, substitution or duplication in the amino acid sequence of the VH FWR.

In a certain embodiment of the present invention, the VL comprises an amino acid sequence set forth in any one of SEQ ID NOs: 151-167 or mutations thereof. In a certain embodiment of the present invention, the VH comprises an amino acid sequence set forth in any one of SEQ ID NOs: 132-149 or mutations thereof.

In a certain preferred embodiment, the VL comprises an amino acid sequence set forth in SEQ ID NO: 161, and the VH comprises an amino acid sequence set forth in SEQ ID NO: 143. In a certain preferred embodiment, the VL comprises an amino acid sequence set forth in SEQ ID NO: 152, and the VH comprises an amino acid sequence set forth in SEQ ID NO: 133. In a certain preferred embodiment, the VL comprises an amino acid sequence set forth in SEQ ID NO: 152, and the VH comprises an amino acid sequence set forth in SEQ ID NO: 146. In a certain preferred embodiment, the VL comprises an amino acid sequence set forth in SEQ ID NO: 164, and the VH comprises an amino acid sequence set forth in SEQ ID NO: 146. In a certain preferred embodiment, the VL comprises an amino acid sequence set forth in SEQ ID NO: 165, and the VH comprises an amino acid sequence set forth in SEQ ID NO: 146. In a certain preferred embodiment, the VL comprises an amino acid sequence set forth in SEQ ID NO: 152, and the VH comprises an amino acid sequence set forth in SEQ ID NO: 147. In a certain preferred embodiment, the VL comprises an amino acid sequence set forth in SEQ ID NO: 164, and the VH comprises an amino acid sequence set forth in SEQ ID NO: 147. In a certain preferred embodiment, the VL comprises an amino acid sequence set forth in SEQ ID NO: 165, and the VH comprises an amino acid sequence set forth in SEQ ID NO: 147. In a certain preferred embodiment, the VL comprises an amino acid sequence set forth in SEQ ID NO: 164, and the VH comprises an amino acid sequence set forth in SEQ ID NO: 133. In a certain preferred embodiment, the VL comprises an amino acid sequence set forth in SEQ ID NO: 165, and the VH comprises an amino acid sequence set forth in SEQ ID NO: 133. In a certain preferred embodiment, the VL comprises an amino acid sequence set forth in SEQ ID NO: 156, and the VH comprises an amino acid sequence set forth in SEQ ID NO: 138. In a certain preferred embodiment, the VL comprises an amino acid sequence set forth in SEQ ID NO: 156, and the VH comprises an amino acid sequence set forth in SEQ ID NO: 148. In a certain preferred embodiment, the VL comprises an amino acid sequence set forth in SEQ ID NO: 166, and the VH comprises an amino acid sequence set forth in SEQ ID NO: 148. In a certain preferred embodiment, the VL comprises an amino acid sequence set forth in SEQ ID NO: 167, and the VH comprises an amino acid sequence set forth in SEQ ID NO: 148. In a certain preferred embodiment, the VL comprises an amino acid sequence set forth in SEQ ID NO: 156, and the VH comprises an amino acid sequence set forth in SEQ ID NO: 149. In a certain preferred embodiment, the VL comprises an amino acid sequence set forth in SEQ ID NO: 166, and the VH comprises an amino acid sequence set forth in SEQ ID NO: 149. In a certain preferred embodiment, the VL comprises an amino acid sequence set forth in SEQ ID NO: 167, and the VH comprises an amino acid sequence set forth in SEQ ID NO: 149. In a certain preferred embodiment, the VL comprises an amino acid sequence set forth in SEQ ID NO: 166, and the VH comprises an amino acid sequence set forth in SEQ ID NO: 138. In a certain preferred embodiment, the VL comprises an amino acid sequence set forth in SEQ ID NO: 167, and the VH comprises an amino acid sequence set forth in SEQ ID NO: 138. In a certain preferred embodiment, the VL comprises an amino acid sequence set forth in SEQ ID NO: 151, and the VH comprises an amino acid sequence set forth in SEQ ID NO: 132. In a certain preferred embodiment, the VL comprises an amino acid sequence set forth in SEQ ID NO: 152, and the VH comprises an amino acid sequence set forth in SEQ ID NO: 134. In a certain preferred embodiment, the VL comprises an amino acid sequence set forth in SEQ ID NO: 153, and the VH comprises an amino acid sequence set forth in SEQ ID NO: 135. In a certain preferred embodiment, the VL comprises an amino acid sequence set forth in SEQ ID NO: 154, and the VH comprises an amino acid sequence set forth in SEQ ID NO: 136. In a certain preferred embodiment, the VL comprises an amino acid sequence set forth in SEQ ID NO: 155, and the VH comprises an amino acid sequence set forth in SEQ ID NO: 137. In a certain preferred embodiment, the VL comprises an amino acid sequence set forth in SEQ ID NO: 157, and the VH comprises an amino acid sequence set forth in SEQ ID NO: 139. In a certain preferred embodiment, the VL comprises an amino acid sequence set forth in SEQ ID NO: 158, and the VH comprises an amino acid sequence set forth in SEQ ID NO: 140. In a certain preferred embodiment, the VL comprises an amino acid sequence set forth in SEQ ID NO: 159, and the VH comprises an amino acid sequence set forth in SEQ ID NO: 141. In a certain preferred embodiment, the VL comprises an amino acid sequence set forth in SEQ ID NO: 160, and the VH comprises an amino acid sequence set forth in SEQ ID NO: 142. In a certain preferred embodiment, the VL comprises an amino acid sequence set forth in SEQ ID NO: 162, and the VH comprises an amino acid sequence set forth in SEQ ID NO: 144. In a certain preferred embodiment, the VL comprises an amino acid sequence set forth in SEQ ID NO: 163, and the VH comprises an amino acid sequence set forth in SEQ ID NO: 145. The above-mentioned mutation is one or more amino acid residue deletions, substitutions or additions occurring in the amino acid sequence of the VH and/or VL, and the amino acid sequence having the mutation has at least 85% sequence identity to the amino acid sequence of the VH and/or VL, and maintains or improves the binding of the antibody to B7-H7; the at least 85% sequence identity is preferably at least 90% sequence identity, more preferably at least 95%, 96%, 97% or 98% sequence identity, most preferably at least 99% sequence identity.

Preferably, the anti-B7-H7 antibody or the antigen-binding fragment thereof of the present invention can be a full-length antibody, Fab, Fab′, F(ab′)₂, Fv, preferably scFv, a bispecific antibody, a multispecific antibody, a heavy chain antibody or a single domain antibody, or a monoclonal antibody or a polyclonal antibody prepared from the above-mentioned antibodies.

In the present application, the “Fab fragment” consists of one light chain and the CH1 and variable region of one heavy chain. The heavy chain of a Fab molecule cannot form a disulfide bond with another heavy chain molecule. The “Fc” region contains two heavy chain fragments comprising the CH1 and CH2 domains of an antibody. The two heavy chain fragments are held together by two or more disulfide bonds and by hydrophobic interactions of the CH3 domains. The “Fab fragment” contains one light chain and a portion of a heavy chain that comprises the VH domain and the CH1 domain and the region between the CH1 and CH2 domains, whereby an interchain disulfide bond can be formed between the two heavy chains of two Fab′ fragments to form a F(ab′)₂ molecule. The “F(ab′)₂ fragment” contains two light chains and two heavy chains that comprise a portion of the constant region between the CH1 and CH2 domains, whereby an interchain disulfide bond is formed between the two heavy chains. Therefore, the F(ab′)₂ fragment consists of two Fab′ fragments held together via a disulfide bond between the two heavy chains. The term “Fv” refers to an antibody fragment consisting of VL and VH domains of a single arm of an antibody, but lacking constant regions.

In the present application, the scFv (single chain antibody fragment) can be a conventional single chain antibody in the art, which comprises a heavy chain variable region, a light chain variable region, and a short peptide of 15 to 20 amino acids. The VL and VH domains are paired via a linker which enables them to be produced as a single polypeptide chain, thereby forming a monovalent molecule [see, e.g., Bird et al., Science 242:423-426 (1988) and Huston et al., Proc. Natl. Acad. Sci. USA 85:5879-5883 (1988)]. Such scFv molecules may have a general structure of NH2-VL-linker-VH—COOH or NH2-VH-linker-VL-COOH. Suitable linkers in the prior art consist of a repetitive G4S amino acid sequence or a variant thereof. For example, a linker having an amino acid sequence (G4S)₄ or (G4S)₃ can be used, but variants thereof may also be used.

In the present application, the term “multispecific antibody” is used in its broadest sense to encompass antibodies with multi-epitope specificities. These multispecific antibodies include, but are not limited to: antibodies comprising a heavy chain variable region (VH) and a light chain variable region (VL), wherein the VH-VL unit has multi-epitope specificity; antibodies having two or more VL and VH regions, wherein each VH-VL unit binds to a different target or a different epitope of the same target; antibodies having two or more single variable regions, wherein each single variable region binds to a different target or a different epitope of the same target; and full-length antibodies, antibody fragments, bispecific antibodies (diabodies), triabodies, antibody fragments that are covalently or non-covalently linked together, etc.

In the present application, the monoclonal antibody or mAb or Ab refers to an antibody obtained from a single clonal cell strain, wherein the cell strain is not limited to eukaryotic, prokaryotic or phage clonal cell strains.

In the present application, the “heavy chain antibody” refers to an antibody comprising only one heavy chain variable region (VHH) and two conventional CH2 and CH3 regions, also known as HCAb.

The “single domain antibody”, also known as “nanobody”, refers to a VHH structure cloned from a heavy chain antibody, which is the smallest unit known that binds to an antigen of interest.

In a certain embodiment of the present invention, the anti-B7-H7 antibody or the antigen-binding fragment thereof is a full-length antibody comprising an antibody heavy chain constant region and an antibody light chain constant region. Preferably, the heavy chain constant region is selected from hIgG1, hIgG2, hIgG3 or hIgG4, and the light chain constant region is selected from a kappa chain or a lamda chain of a human antibody. More preferably, the heavy chain constant region is hIgG1, and the light chain constant region is a kappa chain of a human antibody.

In a certain preferred embodiment, the full-length antibody comprises a heavy chain and a light chain; wherein the heavy chain comprises an amino acid sequence set forth in any one of SEQ ID NOs: 169-190 or mutations thereof; and/or the light chain comprises an amino acid sequence set forth in any one of SEQ ID NOs: 192-208 or mutations thereof.

In a certain preferred embodiment, the full-length antibody comprises a heavy chain and a light chain; wherein the heavy chain comprises an amino acid sequence set forth in SEQ ID NO: 186, and the light chain comprises an amino acid sequence set forth in SEQ ID NO: 202. In a certain preferred embodiment, the full-length antibody comprises a heavy chain and a light chain; wherein the heavy chain comprises an amino acid sequence set forth in SEQ ID NO: 180, and the light chain comprises an amino acid sequence set forth in SEQ ID NO: 202. In a certain preferred embodiment, the full-length antibody comprises a heavy chain and a light chain; wherein the heavy chain comprises an amino acid sequence set forth in SEQ ID NO: 184, and the light chain comprises an amino acid sequence set forth in SEQ ID NO: 193. In a certain preferred embodiment, the full-length antibody comprises a heavy chain and a light chain; wherein the heavy chain comprises an amino acid sequence set forth in SEQ ID NO: 170, and the light chain comprises an amino acid sequence set forth in SEQ ID NO: 193. In a certain preferred embodiment, the full-length antibody comprises a heavy chain and a light chain; wherein the heavy chain comprises an amino acid sequence set forth in SEQ ID NO: 187, and the light chain comprises an amino acid sequence set forth in SEQ ID NO: 193. In a certain preferred embodiment, the full-length antibody comprises a heavy chain and a light chain; wherein the heavy chain comprises an amino acid sequence set forth in SEQ ID NO: 187, and the light chain comprises an amino acid sequence set forth in SEQ ID NO: 205. In a certain preferred embodiment, the full-length antibody comprises a heavy chain and a light chain; wherein the heavy chain comprises an amino acid sequence set forth in SEQ ID NO: 187, and the light chain comprises an amino acid sequence set forth in SEQ ID NO: 206. In a certain preferred embodiment, the full-length antibody comprises a heavy chain and a light chain; wherein the heavy chain comprises an amino acid sequence set forth in SEQ ID NO: 188, and the light chain comprises an amino acid sequence set forth in SEQ ID NO: 193. In a certain preferred embodiment, the full-length antibody comprises a heavy chain and a light chain; wherein the heavy chain comprises an amino acid sequence set forth in SEQ ID NO: 188, and the light chain comprises an amino acid sequence set forth in SEQ ID NO: 205. In a certain preferred embodiment, the full-length antibody comprises a heavy chain and a light chain; wherein the heavy chain comprises an amino acid sequence set forth in SEQ ID NO: 188, and the light chain comprises an amino acid sequence set forth in SEQ ID NO: 206. In a certain preferred embodiment, the full-length antibody comprises a heavy chain and a light chain; wherein the heavy chain comprises an amino acid sequence set forth in SEQ ID NO: 184, and the light chain comprises an amino acid sequence set forth in SEQ ID NO: 205. In a certain preferred embodiment, the full-length antibody comprises a heavy chain and a light chain; wherein the heavy chain comprises an amino acid sequence set forth in SEQ ID NO: 184, and the light chain comprises an amino acid sequence set forth in SEQ ID NO: 206. In a certain preferred embodiment, the full-length antibody comprises a heavy chain and a light chain; wherein the heavy chain comprises an amino acid sequence set forth in SEQ ID NO: 185, and the light chain comprises an amino acid sequence set forth in SEQ ID NO: 197. In a certain preferred embodiment, the full-length antibody comprises a heavy chain and a light chain; wherein the heavy chain comprises an amino acid sequence set forth in SEQ ID NO: 175, and the light chain comprises an amino acid sequence set forth in SEQ ID NO: 197. In a certain preferred embodiment, the full-length antibody comprises a heavy chain and a light chain; wherein the heavy chain comprises an amino acid sequence set forth in SEQ ID NO: 189, and the light chain comprises an amino acid sequence set forth in SEQ ID NO: 197. In a certain preferred embodiment, the full-length antibody comprises a heavy chain and a light chain; wherein the heavy chain comprises an amino acid sequence set forth in SEQ ID NO: 189, and the light chain comprises an amino acid sequence set forth in SEQ ID NO: 207. In a certain preferred embodiment, the full-length antibody comprises a heavy chain and a light chain; wherein the heavy chain comprises an amino acid sequence set forth in SEQ ID NO: 189, and the light chain comprises an amino acid sequence set forth in SEQ ID NO: 208. In a certain preferred embodiment, the full-length antibody comprises a heavy chain and a light chain; wherein the heavy chain comprises an amino acid sequence set forth in SEQ ID NO: 190, and the light chain comprises an amino acid sequence set forth in SEQ ID NO: 197. In a certain preferred embodiment, the full-length antibody comprises a heavy chain and a light chain; wherein the heavy chain comprises an amino acid sequence set forth in SEQ ID NO: 190, and the light chain comprises an amino acid sequence set forth in SEQ ID NO: 207. In a certain preferred embodiment, the full-length antibody comprises a heavy chain and a light chain; wherein the heavy chain comprises an amino acid sequence set forth in SEQ ID NO: 190, and the light chain comprises an amino acid sequence set forth in SEQ ID NO: 208. In a certain preferred embodiment, the full-length antibody comprises a heavy chain and a light chain; wherein the heavy chain comprises an amino acid sequence set forth in SEQ ID NO: 185, and the light chain comprises an amino acid sequence set forth in SEQ ID NO: 207. In a certain preferred embodiment, the full-length antibody comprises a heavy chain and a light chain; wherein the heavy chain comprises an amino acid sequence set forth in SEQ ID NO: 185, and the light chain comprises an amino acid sequence set forth in SEQ ID NO: 208. In a certain preferred embodiment, the full-length antibody comprises a heavy chain and a light chain; wherein the heavy chain comprises an amino acid sequence set forth in SEQ ID NO: 169, and the light chain comprises an amino acid sequence set forth in SEQ ID NO: 192. In a certain preferred embodiment, the full-length antibody comprises a heavy chain and a light chain; wherein the heavy chain comprises an amino acid sequence set forth in SEQ ID NO: 171, and the light chain comprises an amino acid sequence set forth in SEQ ID NO: 193. In a certain preferred embodiment, the full-length antibody comprises a heavy chain and a light chain; wherein the heavy chain comprises an amino acid sequence set forth in SEQ ID NO: 172, and the light chain comprises an amino acid sequence set forth in SEQ ID NO: 194. In a certain preferred embodiment, the full-length antibody comprises a heavy chain and a light chain; wherein the heavy chain comprises an amino acid sequence set forth in SEQ ID NO: 173, and the light chain comprises an amino acid sequence set forth in SEQ ID NO: 195. In a certain preferred embodiment, the full-length antibody comprises a heavy chain and a light chain; wherein the heavy chain comprises an amino acid sequence set forth in SEQ ID NO: 174, and the light chain comprises an amino acid sequence set forth in SEQ ID NO: 196. In a certain preferred embodiment, the full-length antibody comprises a heavy chain and a light chain; wherein the heavy chain comprises an amino acid sequence set forth in SEQ ID NO: 176, and the light chain comprises an amino acid sequence set forth in SEQ ID NO: 198. In a certain preferred embodiment, the full-length antibody comprises a heavy chain and a light chain; wherein the heavy chain comprises an amino acid sequence set forth in SEQ ID NO: 177, and the light chain comprises an amino acid sequence set forth in SEQ ID NO: 199. In a certain preferred embodiment, the full-length antibody comprises a heavy chain and a light chain; wherein the heavy chain comprises an amino acid sequence set forth in SEQ ID NO: 178, and the light chain comprises an amino acid sequence set forth in SEQ ID NO: 200. In a certain preferred embodiment, the full-length antibody comprises a heavy chain and a light chain; wherein the heavy chain comprises an amino acid sequence set forth in SEQ ID NO: 179, and the light chain comprises an amino acid sequence set forth in SEQ ID NO: 201. In a certain preferred embodiment, the full-length antibody comprises a heavy chain and a light chain; wherein the heavy chain comprises an amino acid sequence set forth in SEQ ID NO: 181, and the light chain comprises an amino acid sequence set forth in SEQ ID NO: 203. In a certain preferred embodiment, the full-length antibody comprises a heavy chain and a light chain; wherein the heavy chain comprises an amino acid sequence set forth in SEQ ID NO: 182, and the light chain comprises an amino acid sequence set forth in SEQ ID NO: 204. The above-mentioned mutation is one or more amino acid residue deletions, substitutions or additions occurring in the amino acid sequence of a heavy chain and/or a light chain, and the amino acid sequence having the mutation has at least 85% sequence identity to the amino acid sequence of the heavy chain and/or the light chain, and maintains or improves the binding of the antibody to B7-H7; the at least 85% sequence identity is preferably at least 90% sequence identity; more preferably at least 95% sequence identity; most preferably at least 99% sequence identity.

In order to solve the above-mentioned technical problem, a second aspect of the present invention provides an isolated nucleic acid encoding the anti-B7-H7 antibody or the antigen-binding fragment thereof according to the first aspect of the present invention.

The method for preparing the nucleic acid is a conventional preparation method in the art, which method preferably comprises the following steps: obtaining the nucleic acid molecule encoding the above-mentioned antibody by gene cloning technique, or obtaining the nucleic acid molecule encoding the above-mentioned antibody by artificial complete sequence synthesis. It is known to those skilled in the art that a substitution, deletion, alteration, insertion or addition can be appropriately introduced into a base sequence encoding the amino acid sequence of the above-mentioned antibody to provide a homolog of a polynucleotide. The homolog of the polynucleotide of the present invention can be prepared by the substitution, deletion or addition of one or more bases of the gene encoding the antibody sequence within a range of maintaining the activity of the antibody.

In order to solve the above-mentioned technical problem, a third aspect of the present invention provides a recombinant expression vector comprising the isolated nucleic acid according to the second aspect of the present invention.

The recombinant expression vector can be obtained by conventional methods in the art, namely, the recombinant expression vector is constructed by linking the nucleic acid molecule of the present application to various expression vectors. The expression vectors are various conventional vectors in the art, as long as they are capable of carrying the aforementioned nucleic acid molecules. Preferably, the expression vector comprises a eukaryotic cell expression vector and/or a prokaryotic cell expression vector.

In order to solve the above-mentioned technical problem, a fourth aspect of the present invention provides a transformant comprising the recombinant expression vector according to the third aspect of the present invention.

The method for preparing the transformant can be a conventional preparation method in the art, for example, the transformant is prepared by transforming the above-mentioned expression vector into a host cell. The host cell of the transformant is various conventional host cells in the art, as long as it meets the following requirements: in the host cell, the above-mentioned recombinant expression vector can stably replicate itself and the nucleic acid carried thereby can be efficiently expressed. Preferably, the host cell is a prokaryotic cell and/or a eukaryotic cell, the prokaryotic cell is preferably an E. coli cell such as a TG1 cell or a BL21 cell (expressing a single chain antibody or a Fab antibody), and the eukaryotic cell is preferably a HEK293 cell or a CHO cell (expressing a full-length IgG antibody). A preferred recombinant expression transformant of the present invention can be obtained by transforming the aforementioned recombinant expression plasmid into a host cell. The transformation method is a conventional transformation method in the art, preferably a chemical transformation method, a heat shock method or an electroporation method.

In order to solve the above-mentioned technical problem, a fifth aspect of the present invention provides a chimeric antigen receptor comprising the anti-B7-H7 antibody or the antigen-binding fragment thereof according to the first aspect of the present invention.

In order to solve the above-mentioned technical problem, a sixth aspect of the present invention provides a genetically modified cell comprising the chimeric antigen receptor according to the fifth aspect of the present invention.

Preferably, the genetically modified cell is a eukaryotic cell, preferably an isolated human cell; more preferably, the genetically modified cell is an immune cell such as a T cell, or an NK cell.

In order to solve the above-mentioned technical problem, a seventh aspect of the present invention provides a method for preparing an anti-B7-H7 antibody or an antigen-binding fragment thereof, which method comprises the following steps: culturing the transformant according to the fourth aspect of the present invention, and obtaining the anti-B7-H7 antibody or the antigen-binding fragment thereof from the culture.

In order to solve the above-mentioned technical problem, an eighth aspect of the present invention provides an antibody-drug conjugate comprising an antibody moiety and a conjugated moiety, wherein the antibody moiety comprises the anti-B7-H7 antibody or the antigen-binding fragment thereof according to the first aspect of the present invention, the conjugated moiety includes, but is not limited to, a detectable marker, a drug, a toxin, a cytokine, a radionuclide, an enzyme, or a combination thereof, and the antibody moiety can be conjugated to the conjugated moiety, for example, via a chemical bond or a linker.

In order to solve the above-mentioned technical problem, a ninth aspect of the present invention provides a pharmaceutical composition comprising the anti-B7-H7 antibody or the antigen-binding fragment thereof according to the first aspect of the present invention, the chimeric antigen receptor according to the fifth aspect of the present invention, the genetically modified cell according to the sixth aspect of the present invention, and/or the antibody-drug conjugate according to the eighth aspect of the present invention.

The pharmaceutical composition of the present invention can be made into various dosage forms as needed, and can be administered by a physician at a dose that is beneficial to a patient based on the type, age, body weight and general disease condition of the patient, the mode of administration, etc. Preferably, the pharmaceutical composition is in a liquid dosage form, a gaseous dosage form, a solid dosage form and a semi-solid dosage form.

Preferably, the pharmaceutical composition can be administered via oral administration, injection administration, nasal administration, transdermal administration or mucosal administration.

Preferably, the pharmaceutical composition further comprises other anti-tumor antibodies as active ingredients.

Preferably, the pharmaceutical composition further comprises a pharmaceutically acceptable carrier. More preferably, the pharmaceutical composition comprises 0.01% to 99.99% of the above-mentioned anti-B7-H7 antibody or the antigen-binding fragment thereof, the chimeric antigen receptor, the genetically modified cell or the antibody-drug conjugate, and 0.01% to 99.99% of a pharmaceutical carrier, and the percentage is mass percentage of the pharmaceutical composition.

The dose level for administration of the pharmaceutical composition of the present invention can be adjusted according to the amount of the composition that can effect the desired diagnostic or therapeutic effect. The administration regimen may also be a single injection or multiple injections, or adjusted. The dose level and regimen selected are reasonably adjusted depending on a variety of factors including the activity, stability (i.e., half-life), preparation and route of administration of the pharmaceutical composition, combinations with other drugs or treatments, a disease or disorder to be detected and/or treated, the health status, prior medical history of a subject to be treated.

The therapeutically effective dose of the pharmaceutical composition of the present invention may initially be estimated in cell culture experiments or animal models such as rodents, rabbits, dogs, pigs and/or primates. Animal models can also be used to determine appropriate concentration ranges and routes of administration. The resulting concentration ranges and routes of administration in the animal models can subsequently be used to determine useful doses and route of administration in humans. In general, the determination and adjustment of effective amount or dose to be administered and the evaluation of when and how such adjustment is to be made are known to those skilled in the art.

For additional guidance on preparations, doses, administration regimens and measurable therapeutic effects, see Berkow et al., (2000), The Merck Manual of Medical Information and Merck&Co. Inc., Whitehouse Station, New Jersey; Ebadi (1998) CRC Desk Reference of Clinical Pharmacology, etc.

In addition, it should be understood by those skilled in the art that, the pharmaceutical composition may further comprise a combined therapeutic agent (i.e., used in combination therapy), the combined therapeutic agent including, but not limited to, a chemotherapeutic agent, a radiotherapeutic agent, an immunosuppressant, and a cytotoxic drug. For a combination therapy with a combined therapeutic agent, the above-mentioned antibody or the antigen-binding fragment thereof, the chimeric antigen receptor, the genetically modified cell, the antibody-drug conjugate, etc., and additional therapeutic or diagnostic agents can each be used as a single agent over any period suitable for performing the intended treatment or diagnosis. Therefore, these single agents can be administered substantially simultaneously (i.e., either as a single preparation or within minutes or hours) or sequentially in succession, depending on the actual requirements at the time of the particular use.

In order to solve the above-mentioned technical problem, a tenth aspect of the present invention provides a kit comprising the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, and/or the pharmaceutical composition according to the ninth aspect of the present invention.

Preferably, the kit further comprises (i) a device for administering the antibody or the antigen-binding fragment thereof, or the chimeric antigen receptor, or the genetically modified cell, or the antibody-drug conjugate, or the pharmaceutical composition; and/or (ii) instructions for use.

In order to solve the above-mentioned technical problem, an eleventh aspect of the present invention provides the use of the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, the pharmaceutical composition according to the ninth aspect of the present invention, and/or the kit according to the tenth aspect of the present invention in the preparation of a drug for the treatment and/or prevention of a cancer.

Preferably, the cancer is selected from one or more of the group consisting of lung cancer, breast cancer, gastric cancer, small intestine cancer, colon cancer, rectal cancer, pancreatic cancer, kidney cancer, bladder cancer, osteosarcoma, etc.

In order to solve the above-mentioned technical problem, a twelfth aspect of the present invention provides an administration device comprising the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, the pharmaceutical composition according to the ninth aspect of the present invention, and/or the kit according to the tenth aspect of the present invention.

Preferably, the administration device further comprises a component, such as a syringe, an infusion device or an implantable administration device, that accommodates the anti-B7-H7 antibody or the antigen-binding fragment thereof, the chimeric antigen receptor, the genetically modified cell, the antibody-drug conjugate, the pharmaceutical composition, and/or the kit, or that administers the anti-B7-H7 antibody or the antigen-binding fragment thereof, the chimeric antigen receptor, the genetically modified cell, the antibody-drug conjugate, the pharmaceutical composition, and/or the kit to a subject.

In order to solve the above-mentioned technical problem, a thirteenth aspect of the present invention provides a method for detecting B7-H7, which method comprises the step of performing detection using the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, the pharmaceutical composition according to the ninth aspect of the present invention, and/or the kit according to the tenth aspect of the present invention.

Preferably, the detection is for non-diagnostic and/or therapeutic purposes, such as for the detection of B7-H7, etc. in basic research and development.

In order to solve the above-mentioned technical problem, the present invention further provides the use of the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, the pharmaceutical composition according to the ninth aspect of the present invention, and/or the kit according to the tenth aspect of the present invention in the prevention and/or treatment of a cancer. Preferably, the cancer is as described in the eleventh aspect of the present invention.

In order to solve the above-mentioned technical problem, the present invention further provides a method for treating and/or preventing a B7-H7-related disease or disorder (such as a cancer, preferably a cancer according to the eleventh aspect of the present invention), which method comprises treating a patient in need thereof by administering to the patient in need thereof a therapeutically effective amount of the antibody or the antigen-binding fragment thereof according to the first aspect of the present invention, the chimeric antigen receptor according to the fifth aspect of the present invention, the genetically modified cell according to the sixth aspect of the present invention, the antibody-drug conjugate according to the eighth aspect of the present invention, the pharmaceutical composition according to the ninth aspect of the present invention, and/or the kit according to the tenth aspect of the present invention.

In the present application, unless otherwise defined, the scientific and technical terms used in the present application have the meanings commonly understood by those skilled in the art. Moreover, laboratory operation steps of cell culture, molecular genetics, nucleic acid chemistry, and immunology used in the present application are all conventional steps widely used in the corresponding art. Meanwhile, for better understanding of the present invention, definitions and explanations of related terms are provided below.

Three-letter codes and single-letter codes of amino acids used in the present invention are as known to those skilled in the art, or as described in J. Biol. Chem, 243, p 3558 (1968).

As used in the present application, the term “include” or “comprise” is intended to indicate that compositions and methods include the elements described but do not exclude other elements, so is the term “consisting of” as understood according to the context.

The term “antibody” of the present application can include an immunoglobulin, which is a tetrapeptide chain structure composed of two identical heavy chains and two identical light chains linked via inter-chain disulfide bonds. Since the amino acid composition and arrangement sequence of the heavy chain constant region of an immunoglobulin are different, the antigenicity of the immunoglobulin is also different. Accordingly, immunoglobulin can be divided into five classes, or isotypes of immunoglobulins, namely IgM, IgD, IgG, IgA and IgE, and the corresponding heavy chains thereof are a u chain, a δ chain, a γ chain, an α chain and an ε chain, respectively. The same class of Ig can also be divided into different subclasses according to the differences in the amino acid composition of the hinge region thereof and the number and position of the disulfide bonds of the heavy chain. For example, IgG can be divided into IgG1, lgG2, IgG3, and IgG4. The light chains are divided into a kappa chain or a lamda chain by the difference in the constant region. Each Ig class of the five Ig classes can have either a kappa chain or a lamda chain.

In the present invention, the antibody light chain variable region of the present application may further comprise a light chain constant region, and the light chain constant region comprises a human kappa chain and a human lamda chain or variants thereof. In the present invention, the antibody heavy chain variable region of the present application may further comprise a heavy chain constant region, and the heavy chain constant region comprises human IgG1, IgG2, IgG3, and IgG4, or variants thereof.

Within light and heavy chains, variable regions and constant regions are linked by a “J” region having about 12 or more amino acids, and the heavy chain further comprises a “D” region having about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain, CL. The constant region of an antibody can mediate the binding of an immunoglobulin to a host tissue or factor, including various cells (e.g., effector cells) of the immune system and the first component (C1q) of the classical complement system. The sequences of about 110 amino acids near the N-terminus of the antibody heavy chain and light chain vary greatly and are the variable regions (V regions); the sequences of other amino acids near the C-terminus are relatively stable and are the constant regions (C regions). The variable region includes 3 hypervariable regions (HVR) and 4 framework regions (FWR) with relatively conserved sequences. Three hypervariable regions, also known as complementarity determining regions (CDRs), determine the specificity of the antibody. Each light chain variable region (VL) and heavy chain variable region (VH) consists of 3 CDRs and 4 FWRs, which are arranged in the following order from amino end to carboxyl end: FWR1, CDR1, FWR2, CDR2, FWR3, CDR3, FWR4. The 3 CDRs of the light chain refer to VL CDR1, VL CDR2 and VL CDR3; and the 3 CDRs of the heavy chain refer to VH CDR1, VH CDR2 and VH CDR3.

The term “human antibody” includes antibodies having variable and constant regions of human germline immunoglobulin sequences. The human antibodies of the present invention may include amino acid residues that are not encoded by human germline immunoglobulin sequences (for example, mutations introduced by random or site-specific mutagenesis in vitro or by somatic mutation in vivo). However, the term “human antibody” does not include antibodies in which CDR sequences derived from the germline of another mammalian species (such as mouse) have been grafted onto human backbone sequences (i.e., “humanized antibodies”).

As used in the present application, the term “specific” with respect to an antibody means an antibody that recognizes a specific antigen but does not substantially recognize or bind to other molecules in a sample. For example, an antibody that specifically binds to an antigen from one species can also bind to that antigen from one or more species. However, this interspecies cross-reactivity itself does not affect the classification of antibodies according to specificity. In another example, an antibody that specifically binds to an antigen can also bind to different allelic forms of the antigen. However, this cross-reactivity itself does not affect the classification of antibodies according to specificity. In some cases, the term “specific” or “specific binding” can be used to refer to the interaction of an antibody, a protein or a peptide with a second chemical substance, meaning that the interaction depends on the presence of a particular structure (e.g., an antigenic determinant or epitope) on the chemical substance; for example, antibodies generally recognize and bind to particular protein structures, rather than proteins. If an antibody is specific for epitope “A”, the presence of a molecule containing epitope A (or free, unlabeled A) will reduce the amount of labeled A bound to the antibody in the reaction between a molecule containing the labeled “A” and the antibody.

In the present application, the term “antigen-binding fragment” refers to an antigen-binding fragment of an antibody and an antibody analog, which generally include at least a portion of the antigen-binding region or variable region (for example, one or more CDRs) of a parental antibody. An antibody fragment retains at least some of the binding specificity of the parental antibody. Typically, the antibody fragment retains at least 10% of the binding activity of the parental antibody when the activity is expressed on a molar basis. Preferably, the antibody fragment retains at least 50%, 60%, 70%, 80%, 90%, 95%, 99% or 100% or more of the binding affinity of the parental antibody for the target. Examples of the antigen-binding fragment include, but are not limited to: Fab, Fab′, F(ab′)₂, a Fv fragment, a linear antibody, a single (heavy) chain antibody, a nanobody, a domain antibody, and a multispecific antibody. Engineered antibody variants are reviewed in Holliger and Hudson (2005) Nat. Biotechnol. 23:1126-1136.

The term “chimeric antigen receptor” or “CAR” as used herein refers to one comprising an extracellular domain capable of binding to an antigen (extracellular binding domain), a hinge domain, a transmembrane domain (transmembrane region), and a polypeptide that enables the cytoplasmic signaling to a domain (i.e., intracellular signaling domain). The hinge domain can be considered as part of a system for providing flexibility to the extracellular antigen-binding region. The intracellular signaling domain refers to a protein that transmits information into a cell via a defined signaling pathway by generating second messengers to regulate cell activity, or a protein that acts as an effector in response to such messengers, generating signals that can promote immune effector functions of CAR cells (e.g., CAR-T cells). The intracellular signaling domain comprises a signaling domain and may also comprise a co-stimulatory intracellular domain derived from a co-stimulatory molecule.

The “identity” and “mutation” refer to sequence similarity between two polynucleotide sequences or between two polypeptides. When a position in two compared sequences is occupied by the same base or amino acid monomer subunit, for example each position in two DNA molecules is occupied by adenine, the molecules are homologous at that position. The percent identity between two sequences is a function of the number of matching or homologous positions shared by the two sequences divided by the number of positions compared×100. For example, when sequences are optimally aligned, if 6 out of 10 positions in two sequences match or are homologous, the two sequences are 60% homologous. In general, comparisons are made when aligning two sequences yields the greatest percent identity.

The terms “polypeptide”, “peptide” and “protein” (if single chain) are used interchangeably in the present application. The terms “nucleic acid”, “nucleic acid sequence”, “nucleotide sequence” or “polynucleotide sequence” and “polynucleotide” are used interchangeably.

The term “vector” refers to a nucleic acid delivery vehicle into which a polynucleotide may be inserted. When the vector allows for the expression of the protein encoded by the inserted polynucleotide, the vector is called an expression vector. A vector can be introduced into a host cell by transformation, transduction or transfection, and the genetic substance elements carried thereby can be expressed in the host cell. Vectors are well known to those skilled in the art, and include, but are not limited to: plasmids; phagemids; cosmids; artificial chromosomes, such as yeast artificial chromosomes (YACs), bacterial artificial chromosomes (BACs) or P1-derived artificial chromosomes (PACs); phages such as a lamda phage or M13 phage and animal viruses. The animal viruses that can be used as vectors include, but are not limited to, retroviruses (including lentiviruses), adenoviruses, adeno-associated viruses, herpes viruses (e.g., herpes simplex virus), poxviruses, baculoviruses, papilloma viruses and papovaviruses (such as SV40). A vector can contain a variety of elements that control expression, including, but not limited to: a promoter sequence, a transcription initiation sequence, an enhancer sequence, a selection element, and a reporter gene. In addition, the vector may further contain a replication initiation site.

The expressions “cell” and “cell line” used in the present application are used interchangeably, and all such designations include progeny. The term “host cell” refers to a cell that can be used to introduce a vector, including but not limited to, a prokaryotic cell such as Escherichia coli, a fungal cell such as a yeast cell, or an animal cell such as a fibroblast, a CHO cell, a COS cell, a NSO cell, an HeLa cell, a BHK cell, a HEK 293 cell or a human cell.

The term “transfection” refers to the introduction of an exogenous nucleic acid into a eukaryotic cell. Transfection can be achieved by a variety of means known in the art, including calcium phosphate-DNA co-precipitation, DEAE-dextran-mediated transfection, polybrene-mediated transfection, electroporation, microinjection, liposome fusion, lipofection, protoplast fusion, retroviral infection and biolistics.

The term “immune cell” refers to a cell that can trigger an immune response, and the “immune cell” and other grammatical forms thereof can refer to an immune cell of any origin. The “immune cells” include, for example, white blood cells (leukocytes), lymphocytes (T cells, B cells, natural killer (NK) cells), and bone marrow-derived cells (neutrophils, eosinophils, basophils, monocytes, macrophages, and dendritic cells) derived from hematopoietic stem cells (HSC) produced in the bone marrow. The term “immune cell” can also be human or non-human. For example, the immune cells can be derived from blood, such as autologous T cells, allogeneic T cells, autologous NK cells and allogeneic NK cells, or from cell lines, such as NK cell lines prepared using EBV infection, NK cells induced and differentiated from embryonic stem cells and iPSC, and NK92 cell lines.

As used in the present application, the term “T cell” refers to a type of lymphocyte that matures in the thymus. T cells play an important role in cell-mediated immunity and differ from other lymphocytes (e.g., B cells) in the presence of T cell receptors on the cell surface. “T cells” include all types of immune cells that express CD3, including helper T cells (CD4+ cells), cytotoxic T cells (CD8+ cells), natural killer T cells, regulatory T cells (Treg) and γ-δ T cells. “Cytotoxic cells” include CD8+ T cells, natural killer (NK) cells and neutrophils, which are capable of mediating cytotoxic responses. As used herein, the term “NK cells” refer to a type of lymphocytes that originate from the bone marrow and play an important role in the innate immune system. NK cells provide a rapid immune response against virally infected cells, tumor cells or other stressed cells, even in the absence of antibodies and major histocompatibility complexes on the cell surface.

The “optional”, “any one”, “any” or “any one of” means that the event or circumstance subsequently described may, but need not occur, and the description includes the occasions where the events or circumstances occur or do not occur. For example, “optionally comprising 1 antibody heavy chain variable region” means that an antibody heavy chain variable region of a particular sequence may, but need not be present. As used in the present invention, the “a/an” is used in the present invention to refer to one or more grammatical objects. Unless clearly indicated otherwise in the content, the term “or” is used in the present invention to mean the term “and/or” and is used interchangeably therewith. The “about” and “approximately” should generally mean the degree of an acceptable error of the measured amount in view of the property or accuracy of the measurement. Exemplary degrees of error are generally within 10% thereof and more generally within 5% thereof. The methods and compositions disclosed in the present invention encompass polypeptides and nucleic acids having a specified sequence, a variant sequence, or a sequence substantially identical or similar thereto, e.g., a sequence that is at least 85%, 90%, 95%, 99% or more identical to the sequence specified. In the case of an amino acid sequence, the term “substantially identical” is used in the present invention to refer to a first amino acid sequence.

As used herein, the term “pharmaceutical composition” generally refers to a preparation that is present in a form which allows active ingredients to be biologically effective and that does not contain additional ingredients which would be unacceptably toxic to a subject to which the pharmaceutical composition is administered. The composition is sterile.

As used herein, the term “pharmaceutically acceptable carrier” refers to a carrier that is pharmacologically and/or physiologically compatible with a subject and active ingredients and that is well known in the art (see, for example, Remington's Pharmaceutical Sciences. Edited by Gennaro AR, 19th ed. Pennsylvania: Mack Publishing Company, 1995), including but not limited to: a pH adjusting agent, a surfactant, an adjuvant, an ion strength enhancer, a diluent, a reagent for maintaining the osmotic pressure, an absorption delaying agent, and a preservative. For example, the pH adjusting agent includes, but is not limited to, a phosphate buffer. The surfactant includes, but is not limited to, a cationic, anionic or nonionic surfactant, such as Tween-80. The ion strength enhancer includes, but is not limited to, sodium chloride. The preservative includes, but is not limited to, various antibacterial reagents and antifungal reagents, such as parabens, chlorobutanol, phenol, and sorbic acid. The reagent for maintaining the osmotic pressure includes, but is not limited to, a saccharide, NaCl, and an analog thereof. The absorption delaying agent includes, but is not limited to, monostearate and gelatin. The diluent includes, but is not limited to, water, aqueous buffers (such as buffered saline), alcohols, polyols (such as glycerol), etc. The preservative includes, but is not limited to, various antibacterial reagents and antifungal reagents, such as thiomersal, 2-phenoxyethanol, parabens, chlorobutanol, phenol, and sorbic acid. The “stabilizer” has the meaning that is commonly understood by those skilled in the art and can stabilize the desired activity of active ingredients in a drug. The stabilizer includes, but is not limited to, sodium glutamate, gelatin, SPGA, saccharides (such as sorbitol, mannitol, starch, sucrose, lactose, dextran or glucose), amino acids (such as glutamic acid or glycine), proteins (such as dry whey, albumin or casein), or degradation products thereof (such as lactalbumin hydrolysate).

As used herein, the term EC50 refers to the concentration for 50% of maximal effect, i.e., the concentration that causes 50% of the maximal effect.

As used herein, the terms “cancer” and “cancer patient” are intended to include all types of cancerous neoplasms or tumorigenic processes, metastatic tissues or malignant transformed cells, tissues or organs, regardless of their histopathological type or aggressive phase. Examples include, but are not limited to, solid tumors, hematological cancers, soft tissue tumors, and metastatic lesions.

It should be understood by those skilled in the art that products, such as CAR-T, TCR-T, double antibodies, polyclonal antibodies, ADCs and other product forms, comprising the anti-B7-H7 antibody or the antigen-binding fragment thereof of the present invention should fall within the scope of protection of the present invention.

On the basis of meeting common knowledge in the art, the above-mentioned various preferred conditions can be combined in any form, such that various preferred examples of the present invention are obtained.

Reagents and raw materials used in the present invention are all commercially available.

The present invention has the positive improvement effects as follows. the anti-B7-H7 antibody or the antigen-binding fragment thereof of the present invention has high affinity and high biological activity for B7-H7, and can effectively block the binding of B7-H7 protein to a receptor CD28H thereof and the binding of B7-H7 protein to a receptor KIR3DL3 thereof; furthermore, the anti-B7-H7 antibody or the antigen-binding fragment thereof of the present invention only specifically binds to B7-H7 protein without cross-reacting with other member proteins of the B7 family, and has good stability. In addition, the anti-B7-H7 antibody or the antigen-binding fragment thereof of the present invention shows strong in vivo anti-tumor activity.

### BRIEF DESCRIPTION OF THE DRAWINGS

FIG. 1 shows the binding of anti-B7-H7 initial antibodies to human and cynomolgus monkey B7-H7 on the cell surface and to B7-H7 on the surface of tumor cells HCC827.

FIG. 2 shows the binding of anti-B7-H7 LALA mutation antibodies to tumor cell LS180.

FIG. 3 shows the binding of anti-B7-H7 PTM mutation/LALA mutation antibodies to tumor cell LS180.

FIG. 4 shows the blocking effects of anti-B7-H7 antibodies against the binding of B7-H7 protein to CD28H on the surface of HEK-293 cells.

FIG. 5 shows the blocking effects of anti-B7-H7 antibodies and anti-B7-H7 LALA mutation antibodies against the binding of B7-H7 protein to KIR3DL3 on the surface of CHO-K1 cells.

FIG. 6 shows the blocking effects of anti-B7-H7 antibodies and anti-B7-H7 LALA mutation antibodies against the binding of B7-H7 protein to KIR3DL3 protein.

FIG. 7 shows that anti-B7-H7 antibodies block the immunosuppressive signaling of B7-H7 and thus activate T cells.

FIG. 8 shows that anti-B7-H7 antibodies do not act on B7-H7 negative cells and thus cannot activate T cells.

FIG. 9 shows that anti-B7-H7 antibody stimulates NK cells to kill target cells.

FIG. 10 shows the cross-reactivity of anti-B7-H7 LALA mutation antibodies with other member proteins of the B7 family.

FIG. 11 shows the stability of anti-B7-H7 LALA mutation antibodies in sera.

FIG. 12 shows the anti-tumor effects of anti-B7-H7 antibodies in mouse tumor models.

### DETAILED DESCRIPTION OF EMBODIMENTS

The present invention is further described below by way of examples; however, the present invention is not limited to the scope of the described examples. For the experimental methods in which no specific conditions are specified in the following examples, selections are made according to conventional methods and conditions or according to the product instructions.

### Example 1. Obtaining of anti-B7-H7 antibody molecules

Experimental animals, which could be mice, rats, rabbits, sheep, camels, etc., were immunized with the B7-H7 recombinant protein to obtain antibody molecules that specifically bind to B7-H7. Usually, the resulting antibody molecules are of non-human origin. After obtaining non-human antibodies, these molecules need to be humanized using antibody engineering techniques to reduce immunogenicity and improve druggability. However, the process of humanizing antibodies has its technical complexity, and molecules that have been humanized tend to have reduced affinity for antigens. On the other hand, advances in transgenic technology allows the breeding of genetically engineered mice that carry the human immunoglobulin repertoire and in which their endogenous murine immunoglobulin repertoires are deleted. Harbour H2L2 mice (Harbour antibodies BV) are transgenic mice carrying the human immunoglobulin repertoire, and the antibodies produced by the transgenic mice have a fully human sequence, so there is no need for further humanization.

### Example 1.1. Mouse Immunization

Harbour H2L2 mice were immunized in multiple rounds with a soluble recombinant human B7-H7-ECD-his fusion protein (Sino Biological, #16139-H08H) as an antigen. The antigen protein was mixed with an immunoadjuvant to form an immunogen reagent, and the immunogen reagent was then injected subcutaneously in the inguinal region or injected intraperitoneally. In each round of immunization, each mouse received a total injection dose of 100 microliters. In the first round of immunization, each mouse was immunized with an immunogen reagent prepared by mixing 50 micrograms of the antigen protein with a complete Freund's adjuvant (Sigma, #F5881) at a volume ratio of 1:1. In each subsequent round of booster immunization, each mouse was immunized with an immunogen reagent prepared by mixing 25 micrograms of the antigen protein with Sigma Adjuvant System adjuvant (Sigma, #S6322). The interval between two rounds of booster immunization was at least two weeks, typically with 4 to 5 rounds of booster immunization. The immunization time was on day 0, 14, 28, 42, 56, 70, 84, and 96; and on days 49 and 77, the antibody titer in the sera of mice was measured. Five days before the isolation of splenic B cells in H2L2 mice, the final booster immunization was performed at a dose of 25 micrograms of the antigen protein per mouse.

### Example 1.2. Measurement of Titer in Sera

At specific time points, mouse sera were harvested, the titer of the antibody that binds to B7-H7 protein in the sera was measured by ELISA, and the titer of the antibody that binds to cells overexpressing B7-H7 in the sera was measured by FACS.

In the ELISA, an ELISA plate (corning, 9018) was coated with 1 μg/mL hB7-H7-ECD-hFc protein (Sino Biological, #16139-H02H) at 100 μL/well and incubated overnight at 4° C.; after the plate was rinsed twice, blocking was performed with PBST containing 1% BSA for 2 hours at 37° C.; gradiently diluted sera were added at 100 L/well, and incubation was performed for 1 hour at 37° C.; and after the plate was rinsed 3 times, anti-rat-HRP (sigma, #A5795) at 1:5000 dilution was added at 100 μL/well, and incubation was performed for 30 minutes at 37° C. After the plate was rinsed 3 times, a TMB substrate was added at 100 μL/well, incubation was performed for about 10 minutes, 1 N HCl was added at 50 μL/well to stop color development, and the absorbance at 450 nm was read (Molecular Devices, Plus 384).

In the FACS, gradiently diluted murine sera were incubated with HEK293-B7-H7 cells for 1 hour at 4° C.; and after the cells were washed twice, a secondary antibody anti-rat IgG (H+L) (Life technologies, A11006) was added, incubation was performed for 1 hour at 4° C., and after the cells were washed twice, the cells were resuspended and then detected by a flow cytometer (BD, Flibur). HEK293 cells were used as a background control.

### Example 1.3. Screening of Anti-B7-H7 Antibodies by Hybridoma Technique

Immunized mice with high valency in the sera were selected for one final immunization and the mice were sacrificed, and the splenocytes and SP2/0 myeloma cells (ATCC, CRL-1581) were taken for electrofusion at a cell ratio of 4:1, wherein the electrofusion parameters were V1: 50 V, t1: 15 s, V2: 600 V, t2: 20 μs, t3: 0.5 s, n: 1, t4: 7 s, V+/−: +, fade: on. The cells were resuspended in a DMEM medium containing 20% FBS and HT, and plated at 1×10⁵⁵ cells/100 μL/well, and after 24 hours, a DMEM medium containing 20% FBS and 2× HT was added at 100 μl/well for further culture. The supernatant was subsequently taken, and the titer of the antibody was detected. Generally, 9-15 days after the fusion, the supernatants from the mice immunized with the protein were initially screened using Acumen to detect the binding to HEK-293/huB7-H7 cells; and the positive clones were then further confirmed by ELISA and FACS to detect the ability for binding to the HEK-293 cell strain overexpressing human B7-H7 (HEK-293/huB7-H7) and HEK-293 cell strain overexpressing cynomolgus monkey B7-H7 (HEK-293/cynoB7-H7). Subcloning was further performed in the positive wells by limiting dilution, and screening was then performed by ELISA and FACS. The clones having good binding to human B7-H7 and monkey B7-H7 were selected for sequencing.

### Example 1.4. Sequencing and Sequence Optimization of Anti-B7-H7 Antibodies

The nucleotide sequences encoding the variable domains of the antibody molecules and the corresponding amino acid sequences were obtained by conventional sequencing means. The sequences of 14 monoclonal antibodies were obtained. In this example, the sequences of the variable domains of the anti-B7-H7 monoclonal antibody molecules obtained from the immunized Harbour H2L2 mice were human antibody sequences. The germline gene analysis and post-translational modification (PTM) site analysis of the anti-B7-H7 monoclonal antibody are listed in Table 1-1.

After the translation synthesis of amino acid chains of proteins or polypeptides in cells, chemical modifications are sometimes introduced, called post-translational modifications (PTMs). For antibodies, some of the PTM sites are very conserved. For example, the conserved amino acid asparagine Asn at position 297 (EU numbering) of the constant domain of human lgG1 antibodies is usually glycosylated to form a saccharide chain, and this saccharide chain structure is critical for the antibody structure and related effector functions. However, if a PTM is present in a variable domain of an antibody, particularly in an antigen-binding region (e.g., CDR), the presence of such a PTM can have a greater impact on the binding to an antigen, and can also cause a change in the physicochemical properties of the antibody. For example, glycosylation, deamidation, isomerization, oxidation, etc. may increase the instability or heterogeneity of antibody molecules, thereby increasing the difficulty and risk of antibody development. Avoiding some potential PTMs is thus very important for the development of therapeutic antibodies. With the accumulation of experience, it has been found that some PTMs are highly correlated with the “pattern” of the composition of amino acid sequences, particularly of the composition of adjacent amino acids, which makes it possible to predict potential PTMs from the primary amino acid sequences of proteins. For example, an N-linked glycosylation site can be predicted according the sequence pattern N-x-S/T (asparagine at the first position, any amino acid other than proline at the second position, and serine or threonine at the third position). Amino acid sequence patterns that cause PTMs may be derived from germline gene sequences, for example, pattern NST where human germline gene fragment IGHV3-33 naturally has a glycosylation modification in the FR3 region; and they may also be derived from somatic hypermutation.

The amino acid sequence patterns of PTMs can be disrupted by amino acid mutations, thereby reducing or removing the formation of particular PTMs. Depending on different antibody sequences and different PTM sequence patterns, there are different mutation design methods. One method is to replace the “hot spot” amino acid (such as N or S in the NS pattern) with an amino acid having similar physicochemical properties (e.g., mutation of N to Q). If the PTM sequence pattern is derived from somatic hypermutation and is not present in the germline gene sequences, another method may be to replace the sequence pattern with the corresponding germline gene sequence. In actual operation, multiple mutation design methods may be used for the same PTM sequence pattern.

The sequences of the new antibody molecules resulting from amino acid mutations in the sequences of antibodies PR003249, PR003259 and PR003264 are listed in Table 1-2, wherein PR003181 is a positive control, from patent WO 2014190356.

TABLE 1-1
Germline gene analysis and PTM site analysis of anti-B7-H7 antibodies

| | | | | | | Recombinant |
|---|---|---|---|---|---|---|
| | VH germline | VL germline | | | Recombinant | antibody |
| Clone No. | V gene | V gene | VH PTM | VL PTM | antibody | subtype |
| 13D8D2 | IGHV3- | IGKV1-5*03 | DG (HCDR2), | NS (LCDR3) | PR003248 | Human IgG1 |
| | 33*01 | | NS (HCDR3) | | | |
| 25H3F2 | IGHV3- | IGKV1-5*03 | DG (HCDR2) | NS (LCDR3) | PR003249 | Human IgG1 |
| | 33*01 | | | | | |
| 34G2E6 | IGHV3- | IGKV1-5*03 | DG (HCDR2) | NS (LCDR3) | PR003250 | Human IgG1 |
| | 33*01 | | | | | |
| 29F2C8 | IGHV3- | IGKV3-15*01 | NG (HCDR2), | | PR003251 | Human IgG1 |
| | 33*01 | | DG (HCDR3) | | | |
| 53C12D4 | IGHV3- | IGKV1-5*03 | DG (HCDR2) | NS (LCDR3) | PR003252 | Human IgG1 |
| | 33*01 | | | | | |
| 59D6D2 | IGHV3- | IGKV3-15*01 | NG (HCDR2), | | PR003254 | Human IgG1 |
| | 33*01 | | DG (HCDR3) | | | |
| 68F3A2 | IGHV3- | IGKV1-27*01 | DG (HCDR2) | NS (LCDR3) | PR003259 | Human IgG1 |
| | 33*06 | | | | | |
| 70F3B1 | IGHV3- | IGKV3-15*01 | DG (HCDR2), | | PR003260 | Human IgG1 |
| | 33*01 | | DG (HCDR3) | | | |
| 74A11E4 | IGHV3- | IGKV1-5*03 | DG (HCDR2) | NS (LCDR3) | PR003261 | Human IgG1 |
| | 33*01 | | | | | |
| 77D5A4 | IGHV3- | IGKV3-15*01 | NG (HCDR2), | | PR003262 | Human IgG1 |
| | 33*01 | | DG (HCDR3) | | | |
| 77E6C4 | IGHV3- | IGKV3-15*01 | DG (HCDR2) | | PR003263 | Human IgG1 |
| | 33*01 | | | | | |
| 77G10D3 | IGHV1- | IGKV1-9*01 | | | PR003264 | Human IgG1 |
| | 69*01 | | | | | |
| 78G8B3 | IGHV3- | IGKV3-15*01 | | | PR003265 | Human IgG1 |
| | 33*01 | | | | | |
| 7D3D2 | IGHV3- | IGKV1-5*03 | DG (HCDR2) | NS (LCDR3) | PR003266 | Human IgG1 |
| | 33*01 | | | | | |

TABLE 1-2
Mutation site design of antigen-binding protein sequences

| | | Mutation in | Recombinant | |
|---|---|---|---|---|
| Initial | | variable | antibody | |
| antibody | Variant | region | subtype | Mutation in Fc |
| PR003181 | PR004260 | — | Human IgG1 | LALA mutation |
| PR003249 | PR004261 | — | Human IgG1 | LALA mutation |
| PR003259 | PR004262 | — | Human IgG1 | LALA mutation |
| PR003264 | PR004263 | — | Human IgG1 | LALA mutation |
| PR003249 | PR004479 | H:D54E | Human IgG1 | LALA mutation |
| PR003249 | PR004480 | H:D54E; L:N92Q | Human IgG1 | LALA mutation |
| PR003249 | PR004481 | H:D54E; L:S93A | Human IgG1 | LALA mutation |
| PR003249 | PR004482 | H:G55A | Human IgG1 | LALA mutation |
| PR003249 | PR004483 | H:G55A; L:N92Q | Human IgG1 | LALA mutation |
| PR003249 | PR004484 | H:G55A; L:S93A | Human IgG1 | LALA mutation |
| PR003249 | PR004485 | L:N92Q | Human IgG1 | LALA mutation |
| PR003249 | PR004486 | L:S93A | Human IgG1 | LALA mutation |
| PR003259 | PR004487 | H:D54E | Human IgG1 | LALA mutation |
| PR003259 | PR004488 | H:D54E; L:N92Q | Human IgG1 | LALA mutation |
| PR003259 | PR004489 | H:D54E; L:S93A | Human IgG1 | LALA mutation |
| PR003259 | PR004490 | H:G55A | Human IgG1 | LALA mutation |
| PR003259 | PR004491 | H:G55A; L:N92Q | Human IgG1 | LALA mutation |
| PR003259 | PR004492 | H:G55A; L:S93A | Human IgG1 | LALA mutation |
| PR003259 | PR004493 | L:N92Q | Human IgG1 | LALA mutation |
| PR003259 | PR004494 | L:S93A | Human IgG1 | LALA mutation |

### Example 1.5. Preparation and Physicochemical Property Characterization Analysis of Recombinant Antibodies

### Example 1.5.1. Expression and Purification of Antibodies

In this example, the general method for preparing antibodies using techniques such as mammalian host cells (e.g., human embryonic kidney cells HEK293 or Chinese hamster ovary cells CHO and derivatives thereof), transient transfection expression and affinity capture isolation was described. The present method is suitable for an antibody of interest containing an Fc region; the antibody of interest may be composed of one or more protein polypeptide chains; and the antibody of interest may be derived from one or more expression plasmids.

The amino acid sequences of antibody polypeptide chains are converted into nucleotide sequences by codon optimization; and the nucleotide sequences for encoding are respectively synthesized and cloned onto expression vectors compatible with a host cell. The plasmids encoding the antibody polypeptide chains are simultaneously transfected into a mammalian host cell according to a particular ratio, and the recombinant antibodies having correct folding and polypeptide chain assembly can be obtained using conventional recombinant protein expression and purification techniques. Specifically, FreeStyle™ 293-F cells (Thermo, #R79007) were subjected to scale-up culture in a FreeStyle™ F17 Expression Medium (Thermo, #A1383504). Before transient transfection, the cells were adjusted to a cell concentration of 6-8×10⁵ cells/mL, and cultured in a shaker at 37° C. and 8% CO₂ for 24 hours at a cell concentration of 1.2×10⁶ cells/mL. 30 mL of the cultured cells was prepared. The plasmids encoding the antibody polypeptide chains were mixed according to a certain ratio, a total of 30 μg of plasmids (the ratio of the plasmids to the cells being 1 μg: 1 mL) were dissolved in 1.5 mL of Opti-MEM reduced serum medium (Thermo, #31985088), and the resulting mixture was filtered with a 0.22 μm filter membrane for sterilization. Another 1.5 mL of Opti-MEM was taken and dissolved in 120 μl of 1 mg/mL PEI (Polysciences, #23966-2), and the resulting mixture was left to stand for 5 minutes. The PEI was slowly added to the plasmids, the resulting mixture was incubated at room temperature for 10 minutes, the plasmid and PEI mixed solution was slowly dripped into the culture flask while shaking the culture flask, and cultured in a shaker at 37° C. and 8% CO₂ for 5 days. The cell viability was observed after 5 days. The culture was collected, and centrifuged at a rotary speed of 3300 g for 10 minutes, and then the supernatant was taken; and the supernatant was then centrifuged at a high speed to remove impurities. A gravity column (Bio-Rad, #7311550) containing MabSelect™ (GE Healthcare, #71-5020-91) was equilibrated with a PBS buffer with pH 7.4, that is, the column was rinsed with 2-5 times the column volume of the buffer. The supernatant sample was loaded onto the column; and the column was rinsed with 5-10 times the column volume of PBS buffer, the protein of interest was eluted with 0.1 M glycine with pH 3.5, then adjusted with Tris-HCl with pH 8.0 until neutrality, and finally transferred to a PBS buffer or a buffer containing other components through concentration and liquid exchange by an ultrafiltration tube (Millipore, #UFC901024) to obtain a purified recombinant antibody solution. Finally, the concentration was determined using NanoDrop (Thermo, NanoDrop™ One), and the purified recombinant antibody solution was subpackaged and stored for later use.

### Example 1.5.2. Analysis of Protein Purity and Aggregate by SEC-HPLC

In this example, analytical size exclusion chromatography (SEC) was used to analyze the purity and aggregate form of the protein samples. An analytical chromatographic column TSKgel G3000SWxl (Tosoh Bioscience, #08541, 5 μm, 7.8 mm×30 cm) was connected to a high pressure liquid chromatograph HPLC (Agilent Technologies, Agilent 1260 Infinity II), and equilibrated with a PBS buffer for at least 1 hour at room temperature. An appropriate amount of protein sample (at least 10 μg) was filtered with a 0.22 μm filter membrane and then injected into the system, and the HPLC procedure was set: a PBS buffer was used to make the sample flow through the column at a flow rate of 1.0 mL/min for up to 25 minutes. HPLC would generate an analytical report where the retention times of components having different molecular sizes in the sample were reported.

### Example 1.5.3. Analysis of Protein Purity and Hydrophobicity by HIC-HPLC

Analytical hydrophobic interaction chromatography (HIC) was used to analyze the purity and hydrophobicity of the protein samples. An analytical chromatographic column TSKge1 Butyl-NPR (Tosoh Bioscience, 14947, 4.6 mm×3.5 cm) was connected to a high pressure liquid chromatograph HPLC (Agilent Technologies, Agilent 1260 Infinity II), and equilibrated with a PBS buffer for at least 1 hour at room temperature. The conditions were set up as follows: a linear gradient from 100% mobile phase A (20 mM histidine, 1.8 M ammonium sulfate, pH 6.0) to 100% mobile phase B (20 mM histidine, pH 6.0) over 16 minutes, a flow rate set at 0.7 mL/min, a protein sample concentration of 1 mg/mL, an injection volume of 20 μl, and a detection wavelength of 280 nm. After acquisition, ChemStation software was used to integrate the chromatogram and calculate the relevant data to generate an analytical report where the retention times of components having different molecular sizes in the sample were reported.

### Example 1.5.4. Determination of Thermal Stability of Protein Molecules by DSF

Differential scanning fluorimetry (DSF) is a common high-throughput method for determine the thermal stability of proteins. In the method, a real-time fluorescent quantitative PCR instrument is used to monitor the change in the fluorescence intensity of the dye bound to the unfolded protein molecule to reflect the process of protein denaturation, thereby reflecting the thermal stability of the protein molecule. In this example, the thermal denaturation temperature (Tm) of the protein molecule was determined by DSF. 10 μg of protein was added to a 96-well PCR plate (Thermo, #AB-0700/W), then 2 μl of 100× diluted dye SYPRO™ (Invitrogen, #2008138) was added, and then a buffer was added to achieve a final volume of 40 μl per well. The PCR plate was sealed and placed in a real-time fluorescent quantitative PCR instrument (Bio-Rad CFX96 PCR System), incubation was first performed at 25° C. for 5 minutes, then the temperature gradually increased from 25° C. to 95° C. at a gradient of 0.2° C./0.2 min, and the temperature decreased to 25° C. at the end of the test. The FRET scanning mode was used, Bio-Rad CFX Maestro software was used to perform data analysis, and the Tm of the sample was calculated.

### Example 1.6. Preparation of Fully Human Recombinant Antibodies Against B7-H7

The fully human IgG antibodies against B7-H7 obtained in example 1.6 were prepared and analyzed using the methods of example 1.5. Table 1-3 and Table 1-4 list the transient expression and purification results of small volume expression systems and large volume expression systems, respectively. In addition, the anti-B7-H7 antibody sequence which was obtained from the existing literature (Table 1-5), was used as the control in subsequent experiments.

TABLE 1-3
Expression of anti-B7-H7 antibodies

| | Expression | Yield after | SEC- |
|---|---|---|---|
| | system and | one-step | HPLC |
| Antibody | volume | purification (mg/L) | purity (%) |
| PR003248 | HEK293; 30 ml | 49.9 | 99.2 |
| PR003249 | HEK293; 30 ml | 43.8 | 99.2 |
| PR003250 | HEK293; 30 ml | 41.1 | 98.2 |
| PR003251 | HEK293; 30 ml | 46.8 | 99.9 |
| PR003252 | HEK293; 30 ml | 53.2 | 97.9 |
| PR003254 | HEK293; 30 ml | 61.5 | 99.4 |
| PR003259 | HEK293; 30 ml | 67.8 | 99.6 |
| PR003260 | HEK293; 30 ml | 54.0 | 93.1 |
| PR003261 | HEK293; 30 ml | 43.2 | 94.4 |
| PR003262 | HEK293; 30 ml | 24.3 | 77.7 |
| PR003263 | HEK293; 30 ml | 58.0 | 99.0 |
| PR003264 | HEK293; 30 ml | 59.7 | 100.0 |
| PR003265 | HEK293; 30 ml | 69.9 | 100.0 |
| PR003266 | HEK293; 30 ml | 44.1 | 99.6 |
| PR003181 | Expi-CHOs; 50 ml | 327.66 | 99.54 |
| PR004479 | HEK293; 10 ml | Expressed | |
| | | supernatant only | |
| PR004480 | HEK293; 10 ml | Expressed | |
| | | supernatant only | |
| PR004481 | HEK293; 10 ml | Expressed | |
| | | supernatant only | |
| PR004482 | HEK293; 10 ml | Expressed | |
| | | supernatant only | |
| PR004483 | HEK293; 10 ml | 11.00 | ND |
| PR004484 | HEK293; 10 ml | 14.93 | 96.44% |
| PR004485 | HEK293; 10 ml | Expressed | |
| | | supernatant only | |
| PR004486 | HEK293; 10 ml | Expressed | |
| | | supernatant only | |
| PR004487 | HEK293; 10 ml | Expressed | |
| | | supernatant only | |
| PR004488 | HEK293; 10 ml | Expressed | |
| | | supernatant only | |
| PR004489 | HEK293; 10 ml | Expressed | |
| | | supernatant only | |
| PR004490 | HEK293; 10 ml | Expressed | |
| | | supernatant only | |
| PR004491 | HEK293; 10 ml | 12.77 | 93.06% |
| PR004492 | HEK293; 10 ml | 14.68 | 94.1% |
| PR004493 | HEK293; 10 ml | Expressed | |
| | | supernatant only | |
| PR004494 | HEK293; 10 ml | Expressed | |
| | | supernatant only | |

TABLE 1-4
Expression of anti-B7-H7 antibodies

| | Expression system | Yield after two-step | SEC-HPLC |
|---|---|---|---|
| Antibody | and volume | purification (mg/L) | purity (%) |
| PR004260 | Expi-CHO/1000 ml | 230.18 | 98.09% |
| PR004261 | Expi-CHO/1000 ml | 266.5 | 97.75% |
| PR004262 | Expi-CHO/1000 ml | 256.3 | 100.00% |
| PR004263 | Expi-CHO/1000 ml | 158.3 | 98.80% |

TABLE 1-5
Information about control antibody

| Control | Antibody | |
|---|---|---|
| antibody | number | Description |
| Control | PR003181 | From patent WO 2014190356 |
| antibody 1 | | |

### Example 1.7. Sequence and Numbering of Anti-B7-H7 Antibodies

The amino acid sequences of the CDRs of the antibodies obtained in the present invention are as shown in the table below, and these CDRs are all shown according to the rules of Chothia definition.

TABLE 1-6
Definition method for CDRs of antibodies of the present application

| | Kabat | Chothia | Combined |
|---|---|---|---|
| LCDR1 | L24—L34 | L24—L34 | L24-L34 |
| LCDR2 | L50—L56 | L50—L56 | L50-L56 |
| LCDR3 | L89—L97 | L89—L97 | L89-L97 |
| HCDR1 | H31—H35 | H26—H32 | H26-H35 |
| HCDR2 | H50—H65 | H52—H56 | H50-H65 |
| HCDR3 | H95—H102 | H95—H102 | H95-H102 |

Laa-Lbb may refer to an amino acid sequence from position aa (Chothia numbering) to position bb (Chothia numbering), starting from the N-terminus of an antibody light chain; and Haa-Hbb may refer to an amino acid sequence from position aa (Chothia numbering) to position bb (Chothia numbering), starting from the N-terminus of an antibody heavy chain. For example, L24-L34 may refer to an amino acid sequence from position 24 to position 34 according to Chothia numbering, starting from the N-terminus of an antibody light chain; and H26-H35 may refer to an amino acid sequence from position 26 to position 35 according to Chothia numbering, starting from the N-terminus of an antibody heavy chain. It should be known by those skilled in the art, when Chothia numbering scheme is used to number CDR, there may be cases where insertion sites are present at some locations (see http://bioinf.org.uk/abs/).

Table 1-7-1 and table 1-7-2 list the sequence numbers corresponding to the sequences of the anti-B7-H7 antibody molecules of the present invention and the control antibody molecule.

TABLE 1-7-1
Sequence numbers of anti-B7-H7 antibodies (rules of Chothia definition)

| | Heavy | | | | | | | |
|---|---|---|---|---|---|---|---|---|
| | chain HC | | HCDR1 | Amino | HCDR2 | Amino | HCDR3 | |
| Antibody | (SEQ ID | VH (SEQ | (SEQ ID | acid | (SEQ ID | acid | (SEQ ID | Amino acid |
| number | NO:) | ID NO:) | NO:) | sequence | NO:) | sequence | NO:) | sequence |
| PR003181 | 168 | 131 | 6 | GYTFTSH | 26 | FPGDGS | 56 | NSFYSMDY |
| PR003248 | 169 | 132 | 7 | GFTFSDY | 27 | WYDGSE | 57 | DRNSGALGYFPH |
| PR003249 | 170 | 133 | 7 | GFTFSDY | 28 | WYDGSD | 58 | DRSSGAMGYFVY |
| PR003250 | 171 | 134 | 7 | GFTFSDY | 28 | WYDGSD | 59 | DRSSGAMGYFAY |
| PR003251 | 172 | 135 | 8 | GFTFSRY | 29 | WYNGNN | 60 | DGAYSYGPFDY |
| PR003252 | 173 | 136 | 9 | GFTFSNY | 27 | WYDGSE | 61 | EYSSGWGYFDY |
| PR003254 | 174 | 137 | 10 | GFTFSSY | 29 | WYNGNN | 62 | DGGYSYGPFDY |
| PR003259 | 175 | 138 | 9 | GFTFSNY | 27 | WYDGSE | 63 | DLISGIYYVDH |
| PR003260 | 176 | 139 | 8 | GFTFSRY | 30 | WYDGSK | 62 | DGGYSYGPFDY |
| PR003261 | 177 | 140 | 9 | GFTFSNY | 31 | WYDGTN | 64 | DRNTAMDYFVY |
| PR003262 | 178 | 141 | 8 | GFTFSRY | 32 | WYNGRN | 62 | DGGYSYGPFDY |
| PR003263 | 179 | 142 | 11 | GFTFSYY | 33 | WFDGTN | 65 | GYGSGSYYNYVM |
| | | | | | | | | DA |
| PR003264 | 180 | 143 | 12 | GGTFSSY | 34 | IPLIGT | 66 | DTMVRGVRALD |
| | | | | | | | | Y |
| PR003265 | 181 | 144 | 13 | GFTFSTY | 35 | WYDESN | 67 | LAHDYGGTYFDY |
| PR003266 | 182 | 145 | 7 | GFTFSDY | 36 | WYDGSN | 68 | DRSSGALGYFQH |
| PR004260 | 183 | 131 | 6 | GYTFTSH | 26 | FPGDGS | 56 | NSFYSMDY |
| PR004261 | 184 | 133 | 7 | GFTFSDY | 28 | WYDGSD | 58 | DRSSGAMGYFVY |
| PR004262 | 185 | 138 | 9 | GFTFSNY | 27 | WYDGSE | 63 | DLISGIYYVDH |
| PR004263 | 186 | 143 | 12 | GGTFSSY | 34 | IPLIGT | 66 | DTMVRGVRALD |
| | | | | | | | | Y |
| PR004479 | 187 | 146 | 7 | GFTFSDY | 37 | WYEGSD | 58 | DRSSGAMGYFVY |
| PR004480 | 187 | 146 | 7 | GFTFSDY | 37 | WYEGSD | 58 | DRSSGAMGYFVY |
| PR004481 | 187 | 146 | 7 | GFTFSDY | 37 | WYEGSD | 58 | DRSSGAMGYFVY |
| PR004482 | 188 | 147 | 7 | GFTFSDY | 38 | WYDASD | 58 | DRSSGAMGYFVY |
| PR004483 | 188 | 147 | 7 | GFTFSDY | 38 | WYDASD | 58 | DRSSGAMGYFVY |
| PR004484 | 188 | 147 | 7 | GFTFSDY | 38 | WYDASD | 58 | DRSSGAMGYFVY |
| PR004485 | 184 | 133 | 7 | GFTFSDY | 28 | WYDGSD | 58 | DRSSGAMGYFVY |
| PR004486 | 184 | 133 | 7 | GFTFSDY | 28 | WYDGSD | 58 | DRSSGAMGYFVY |
| PR004487 | 189 | 148 | 9 | GFTFSNY | 39 | WYEGSE | 63 | DLISGIYYVDH |
| PR004488 | 189 | 148 | 9 | GFTFSNY | 39 | WYEGSE | 63 | DLISGIYYVDH |
| PR004489 | 189 | 148 | 9 | GFTFSNY | 39 | WYEGSE | 63 | DLISGIYYVDH |
| PR004490 | 190 | 149 | 9 | GFTFSNY | 40 | WYDASE | 63 | DLISGIYYVDH |
| PR004491 | 190 | 149 | 9 | GFTFSNY | 40 | WYDASE | 63 | DLISGIYYVDH |
| PR004492 | 190 | 149 | 9 | GFTFSNY | 40 | WYDASE | 63 | DLISGIYYVDH |
| PR004493 | 185 | 138 | 9 | GFTFSNY | 27 | WYDGSE | 63 | DLISGIYYVDH |
| PR004494 | 185 | 138 | 9 | GFTFSNY | 27 | WYDGSE | 63 | DLISGIYYVDH |

TABLE 1-7-2
Sequence numbers of anti-B7-H7 antibodies (rules of Chothia definition)

| | Light | | | | | | | |
|---|---|---|---|---|---|---|---|---|
| | chain LC | VL | LCDR1 | | LCDR2 | Amino | LCDR3 | |
| Antibody | (SEQ ID | (SEQ ID | (SEQ ID | Amino acid | (SEQ ID | acid | (SEQ ID | Amino acid |
| number | NO:) | NO:) | NO:) | sequence | NO:) | sequence | NO:) | sequence |
| PR003181 | 191 | 150 | 80 | KSSQSLLNSR | 93 | WASTRES | 110 | KQSYNLRT |
| | | | | TRKNQLA | | | | |
| PR003248 | 192 | 151 | 81 | RASQSINN | 94 | KASSLES | 111 | QQYNSYST |
| | | | | WLA | | | | |
| PR003249 | 193 | 152 | 82 | RASQSISSW | 94 | KASSLES | 112 | QQYNSYSYT |
| | | | | LA | | | | |
| PR003250 | 193 | 152 | 82 | RASQSISSW | 94 | KASSLES | 112 | QQYNSYSYT |
| | | | | LA | | | | |
| PR003251 | 194 | 153 | 83 | RASQSVSSN | 95 | GASTRAT | 113 | QQYNIWPYT |
| | | | | LA | | | | |
| PR003252 | 195 | 154 | 82 | RASQSISSW | 96 | KASSLKS | 114 | LQYNSYYT |
| | | | | LA | | | | |
| PR003254 | 196 | 155 | 84 | RASQSVSSYL | 95 | GASTRAT | 115 | QQYNNWPL |
| | | | | A | | | | T |
| PR003259 | 197 | 156 | 85 | RASQGISNY | 97 | AASTLQS | 116 | QKYNSAPFT |
| | | | | LA | | | | |
| PR003260 | 198 | 157 | 83 | RASQSVSSN | 98 | YASTRAP | 113 | QQYNIWPYT |
| | | | | LA | | | | |
| PR003261 | 199 | 158 | 82 | RASQSISSW | 94 | KASSLES | 117 | QQYNSYSVT |
| | | | | LA | | | | |
| PR003262 | 200 | 159 | 86 | RASQSFSNN | 95 | GASTRAT | 118 | HQYNIWPLT |
| | | | | LA | | | | |
| PR003263 | 201 | 160 | 83 | RASQSVSSN | 99 | GASTGAT | 119 | QQYYKWPLT |
| | | | | LA | | | | |
| PR003264 | 202 | 161 | 87 | RASQGISSYF | 97 | AASTLQS | 120 | QHLNRYPW |
| | | | | A | | | | T |
| PR003265 | 203 | 162 | 88 | RASQSISYNL | 100 | GASIRAT | 121 | QQYNNWPP |
| | | | | A | | | | WT |
| PR003266 | 204 | 163 | 82 | RASQSISSW | 94 | KASSLES | 111 | QQYNSYST |
| | | | | LA | | | | |
| PR004260 | 191 | 150 | 80 | KSSQSLLNSR | 93 | WASTRES | 110 | KQSYNLRT |
| | | | | TRKNQLA | | | | |
| PR004261 | 193 | 152 | 82 | RASQSISSW | 94 | KASSLES | 112 | QQYNSYSYT |
| | | | | LA | | | | |
| PR004262 | 197 | 156 | 85 | RASQGISNY | 97 | AASTLQS | 116 | QKYNSAPFT |
| | | | | LA | | | | |
| PR004263 | 202 | 161 | 87 | RASQGISSYF | 97 | AASTLQS | 120 | QHLNRYPW |
| | | | | A | | | | T |
| PR004479 | 193 | 152 | 82 | RASQSISSW | 94 | KASSLES | 112 | QQYNSYSYT |
| | | | | LA | | | | |
| PR004480 | 205 | 164 | 82 | RASQSISSW | 94 | KASSLES | 122 | QQYQSYSYT |
| | | | | LA | | | | |
| PR004481 | 206 | 165 | 82 | RASQSISSW | 94 | KASSLES | 123 | QQYNAYSYT |
| | | | | LA | | | | |
| PR004482 | 193 | 152 | 82 | RASQSISSW | 94 | KASSLES | 112 | QQYNSYSYT |
| | | | | LA | | | | |
| PR004483 | 205 | 164 | 82 | RASQSISSW | 94 | KASSLES | 122 | QQYQSYSYT |
| | | | | LA | | | | |
| PR004484 | 206 | 165 | 82 | RASQSISSW | 94 | KASSLES | 123 | QQYNAYSYT |
| | | | | LA | | | | |
| PR004485 | 205 | 164 | 82 | RASQSISSW | 94 | KASSLES | 122 | QQYQSYSYT |
| | | | | LA | | | | |
| PR004486 | 206 | 165 | 82 | RASQSISSW | 94 | KASSLES | 123 | QQYNAYSYT |
| | | | | LA | | | | |
| PR004487 | 197 | 156 | 85 | RASQGISNY | 97 | AASTLQS | 116 | QKYNSAPFT |
| | | | | LA | | | | |
| PR004488 | 207 | 166 | 85 | RASQGISNY | 97 | AASTLQS | 124 | QKYQSAPFT |
| | | | | LA | | | | |
| PR004489 | 208 | 167 | 85 | RASQGISNY | 97 | AASTLQS | 125 | QKYNAAPFT |
| | | | | LA | | | | |
| PR004490 | 197 | 156 | 85 | RASQGISNY | 97 | AASTLQS | 116 | QKYNSAPFT |
| | | | | LA | | | | |
| PR004491 | 207 | 166 | 85 | RASQGISNY | 97 | AASTLQS | 124 | QKYQSAPFT |
| | | | | LA | | | | |
| PR004492 | 208 | 167 | 85 | RASQGISNY | 97 | AASTLQS | 125 | QKYNAAPFT |
| | | | | LA | | | | |
| PR004493 | 207 | 166 | 85 | RASQGISNY | 97 | AASTLQS | 124 | QKYQSAPFT |
| | | | | LA | | | | |
| PR004494 | 208 | 167 | 85 | RASQGISNY | 97 | AASTLQS | 125 | QKYNAAPFT |
| | | | | LA | | | | |

### Example 2. Determination of Ability of Anti-B7-H7 Antibodies of Binding to B7-H7 by FACS

This example was intended to study the in vitro activity of H2L2 monoclonal antibodies against human B7-H7 in binding to human/cynomolgus monkey B7-H7. Since homologous molecules of B7-H7 were not present in mice, the detection was not performed. A HEK-293 cell strain (HEK-293/hu B7-H7, Harbour BioMed US, Inc.) overexpressing human B7-H7, a HEK-293 cell strain (HEK-293/cyno B7-H7, Harbour BioMed US, Inc.) overexpressing cynomolgus monkey B7-H7, or cell lines HCC827 (ATCC® CRL-2868) and LS180 (CoBioer Biosciences Co., Ltd., CBP60034) highly expressing human B7-H7 were used for antibody binding experiments at the cellular level. Briefly, the HEK-293/hu B7-H7 cells, HEK-293/cyno B7-H7 cells, HCC827 cells or LS180 cells were digested and resuspended in PBS containing 2% BSA. The cells were adjusted to a cell density of 1×10⁶ cells/mL, respectively. The cells were seeded in a 96-well V-bottom plate (Corning, #3894) at 100 μL/well, and the antibody to be tested at 2× final concentration with 3-fold concentration gradient dilution was then added at 100 μL/well. The cells were incubated at 4° C. for 2 hours in the dark. After the incubation, the cells were rinsed twice by adding pre-chilled PBS containing 2% BSA at 100 μl/well, and centrifuged at 500 g at 4° C. for 5 minutes, and the supernatant was discarded. A fluorescent secondary antibody (Alexa Fluor 488-conjugated AffiniPure Goat Anti-Human IgG, Fcγ Fragment Specific, Jackson, #109-545-098, 1:500 dilution) was added at 100 μl/well, and the cells were incubated at 4° C. for 1 hour in the dark. The cells were washed twice by adding pre-chilled PBS containing 2% BSA at 100 μL/well, and centrifuged at 500 g at 4° C. for 5 minutes, and the supernatant was discarded. Finally, the cells were resuspended in pre-chilled PBS containing 2% BSA at 200 μL/well, and the fluorescence signal values were read using an ACEA Novocyte3000 flow cytometer.

The binding of the antibodies to human B7-H7 and cynomolgus monkey B7-H7 on the cell surface and to B7-H7 on the surface of tumor cells HCC827 and LS180 is summarized as follows (Table 2-1, Table 2-2, and Table 2-3).

TABLE 2-1
Binding of anti-B7-H7 antibodies (initial antibodies) to
human B7-H7 and cynomolgus monkey B7-H7 on the cell surface
and to B7-H7 on the surface of tumor cells HCC827

| | HEK293-hB7-H7 | | HEK293-cynoB7-H7 | | HCC827 | |
|---|---|---|---|---|---|---|
| | Maximum | | Maximum | | Maximum | |
| Antibody | MFI | EC50 (nM) | MFI | EC50 (nM) | MFI | EC50 (nM) |
| PR003248 | 981234 | 1.812 | 698165 | 8.513 | 68608 | 0.03993 |
| PR003249 | 800166 | 0.8811 | 418560 | 0.4117 | 55425 | 0.01507 |
| PR003250 | 822751 | 0.9951 | 404835 | 0.3991 | 68820 | 0.002943 |
| PR003251 | 813378 | 1.068 | 430442 | 0.522 | 53281 | 0.03726 |
| PR003252 | 836597 | 1.043 | 414124 | 0.4294 | 69422 | ~ |
| PR003254 | 762388 | 1.95 | 389065 | 1.241 | 45934 | 0.192 |
| PR003259 | 807637 | 0.945 | 418922 | 0.4211 | 68157 | ~ |
| PR003260 | 806680 | 1.132 | 446853 | 0.5884 | 53349 | 0.04389 |
| PR003261 | 723446 | 0.9412 | 274992 | 0.3259 | 60082 | 0.04449 |
| PR003262 | 821530 | 1.329 | 419256 | 0.59 | 53997 | 0.04101 |
| PR003263 | 762945 | 1.064 | 409860 | 0.5559 | 53208 | 0.07892 |
| PR003264 | 756222 | 1.682 | 289909 | 0.8289 | 56951 | 0.6181 |
| PR003265 | 824473 | 1.244 | 450978 | 0.711 | 51768 | 0.09029 |
| PR003266 | 531300 | 0.6631 | 170315 | 0.211 | 49312 | 0.04772 |
| PR003181 | ~ | ~ | ~ | ~ | 25116 | 2.553 |

~ indicating that weaker binding results in a curve that could not be fitted, so that maximum MFI and EC50 could not be calculated.

TABLE 2-2
Binding of anti-B7-H7 antibodies (variant molecules, LALA
mutation) to B7-H7 on the surface of tumor cells LS180
LS180

| Antibody | Maximum MFI | EC50 (nM) |
|---|---|---|
| PR003181 | ~ | ~ |
| PR003249 | 21042 | 0.035 |
| PR003259 | 20317 | 0.066 |
| PR003264 | 21767 | 2.198 |
| PR004260 | ~ | ~ |
| PR004261 | 19723 | 0.036 |
| PR004262 | 19225 | 0.048 |
| PR004263 | 23985 | 3.075 |

~indicating that weaker binding results in a curve that could not be fitted, so that maximum MFI and EC50 could not be calculated.

TABLE 2-3
Binding of anti-B7-H7 antibodies (variant molecules, PTM
removal) to B7-H7 on the surface of tumor cells LS180
LS180

| Antibody | Maximum MFI | EC50 (nM) |
|---|---|---|
| PR004261 | 26913 | 0.034 |
| PR004262 | 25881 | 0.033 |
| PR004483 | 25060 | 0.044 |
| PR004484 | 24634 | 0.04 |
| PR004491 | 22835 | 0.066 |
| PR004492 | 22862 | 0.052 |

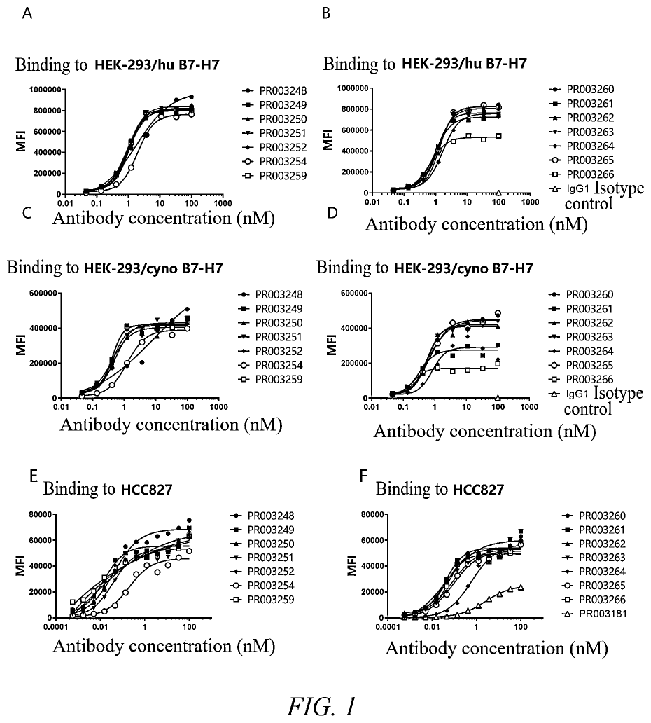

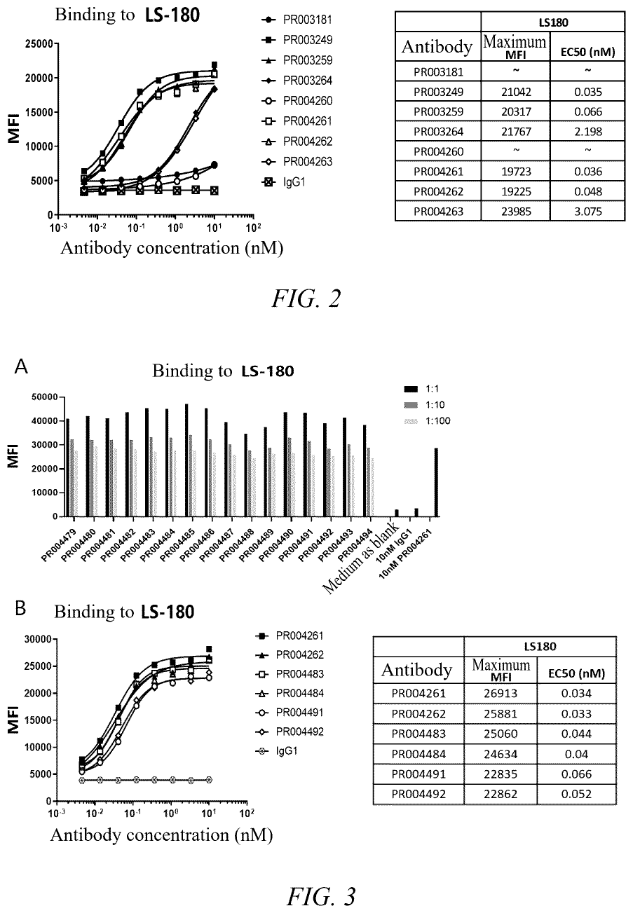

The results of the binding of the initial antibodies to human B7-H7 on the cell surface are as shown in FIG. 1A-1F. The results show that these initial antibodies have good activity of binding to human and cynomolgus monkey B7-H7, and can bind to B7-H7 on the surface of tumor cells. The Fc termini of PR003181, PR003249, PR003259 and PR003264 were engineered with LALA mutations to remove ADCC effects. The results show that the engineered antibodies PR004260, PR004261, PR004262 and PR004263 are consistent with the initial antibodies in the binding activity to cells (FIG. 2). PTM removal was further performed on PR004261 and PR004262 to obtain antibodies PR004479-PR004494. The FACS results show that their binding activity is substantially consistent with that of the initial antibodies (FIG. 3).

### Example 3. Blocking Effect of Antibodies Against the Binding of Human B7-H7 Protein to HEK293 Cells Overexpressing Human CD28H

This example was intended to study the in vitro blocking activity of human anti-B7-H7 antibodies against the binding of human B7-H7 to its receptor CD28H. A HEK-293 cell strain (HEK-293/CD28H, Harbour BioMed US, Inc.) overexpressing human CD28H was used in experiments at the cellular level where human B7-H7/human CD28H binding was blocked. Briefly, the HEK-293/CD28H cells were digested and resuspended in PBS containing 2% BSA. The cells were adjusted to a cell density of 1×10⁶ cells/mL. The cells were seeded in a 96-well V-bottom plate (Corning, Cat #: 3894) at 100 μL/well, and an antigen-binding protein to be tested at 2× final concentration with 3-fold concentration gradient dilution was then added at 100 μl/well, and uniformly mixed, with the maximum final concentration of the antigen-binding protein of 100 nM, a total of 8 concentrations, and hIgG as the control. The cells were incubated at 4° C. for 1 hour in the dark. After the incubation, the cells were centrifuged at 4° C. for 5 minutes, the supernatant was discarded, then a biotinylated human B7-H7 protein (Acro Biosystems, B77-H82F5) with a concentration of 1 μg/mL was added at 50 μL/well, and the cells were incubated at 4° C. for 30 minutes in the dark. The cells were rinsed twice by adding pre-chilled PBS containing 2% BSA at 100 μL/well, and were centrifuged at 500 g at 4° C. for 5 minutes, and the supernatant was discarded. A fluorescent secondary antibody (PE Streptavidin, BD, Cat #: 554061, 1:200) was added at 100 μL/well, and the cells were incubated at 4° C. for 30 minutes in the dark. The cells were washed twice by adding pre-chilled PBS at 200 μL/well, and centrifuged at 500 g at 4° C. for 5 minutes, and the supernatant was discarded. Finally, the cells were resuspended in pre-chilled PBS at 200 μL/well, and the fluorescence signal values were read using an ACEA Novocyte3000 flow cytometer. IC50 was calculated.

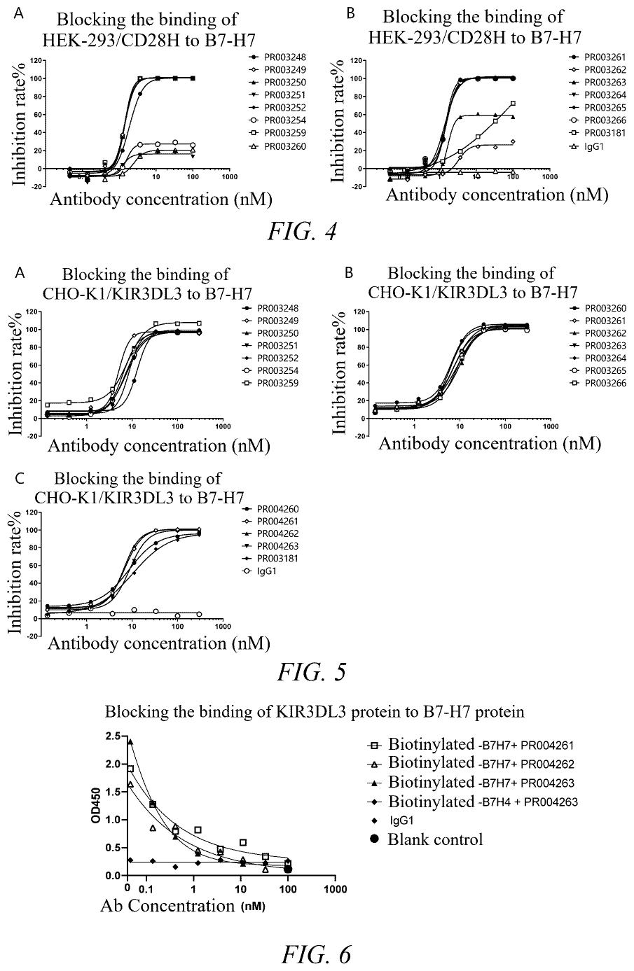

The results are as shown in FIG. 4 and Table 3, indicating that the blocking efficiency of PR003251, PR003254, PR003260, PR003262, PR003263 and PR003181 is acceptable, and the other antibodies can completely block the binding of B7-H7 protein to its receptor CD28H, all of which are superior to the positive control PR003181.

TABLE 3
Blocking effect of antibodies against the binding of human
B7-H7 to human CD28H on the cell surface

| | Maximum | |
|---|---|---|
| Antibody | inhibition rate (%) | IC50 (nM) |
| PR003248 | 100.8 | 1.87 |
| PR003249 | 101.1 | 1.377 |
| PR003250 | 100.6 | 1.405 |
| PR003251 | 16.23 | 1.241 |
| PR003252 | 100.7 | 1.434 |
| PR003254 | 27.44 | 1.589 |
| PR003259 | 101.2 | 1.428 |
| PR003260 | 20.44 | 2.474 |
| PR003261 | 100.4 | 1.493 |
| PR003262 | 26.36 | 3.372 |
| PR003263 | 59.29 | 1.7 |
| PR003264 | 101 | 1.516 |
| PR003265 | 102.1 | 1.388 |
| PR003266 | 100.6 | 1.566 |
| PR003181 | ~ | 71.67 |

### Example 4. Blocking Effect of Antibodies Against the Binding of Human B7-H7 Protein to Human KIR3DL3 Protein and to CHO-K1 Cells Overexpressing KIR3DL3

### Example 4.1. Blocking Effect of Antibodies Against the Binding of Human B7-H7 Protein to KIR3DL3 Expressed on the Cell Surface

This example was intended to study the in vitro blocking activity of human anti-B7-H7 antibodies against the binding of human B7-H7 to its receptor KIR3DL3. A CHO-K1 cell (CHO-K1/KIR3DL3, Harbour BioMed US, Inc.) overexpressing human KIR3DL3 was used in experiments at the cellular level where human B7-H7/human KIR3DL3 binding was blocked. Briefly, the CHO-K1/KIR3DL3 cells were digested and resuspended in PBS containing 2% BSA. The cells were adjusted to a cell density of 1×10⁶ cells/mL. The cells were seeded in a 96-well V-bottom plate (Corning, Cat #: 3894) at 100 μL/well, and an antigen-binding protein to be tested at 2× final concentration with 3-fold concentration gradient dilution was then added at 100 μL/well, and uniformly mixed, with the maximum final concentration of the antigen-binding protein of 100 nM, a total of 8 concentrations, and hIgG as the control. The cells were incubated at 4° C. for 1 hour in the dark. After the incubation, the cells were centrifuged at 4° C. for 5 minutes, the supernatant was discarded, then a biotinylated human B7-H7 protein (Acro Biosystems, B77-H82F5) with a concentration of 1 μg/mL was added at 50 μL/well, and the cells were incubated at 4° C. for 30 minutes in the dark. The cells were rinsed twice by adding pre-chilled PBS containing 2% BSA at 100 μL/well, and were centrifuged at 500 g at 4° C. for 5 minutes, and the supernatant was discarded. A fluorescent secondary antibody (PE Streptavidin, BD, Cat #: 554061, 1:200) was added at 100 μL/well, and the cells were incubated at 4° C. for 30 minutes in the dark. The cells were washed twice by adding pre-chilled PBS at 200 L/well, and centrifuged at 500 g at 4° C. for 5 minutes, and the supernatant was discarded. Finally, the cells were resuspended in pre-chilled PBS at 200 μL/well, and the fluorescence signal values were read using an ACEA Novocyte3000 flow cytometer. IC50 was calculated.

The results are as shown in FIG. 5 and Table 4, indicating that all antibodies, except for the control IgG, are shown to completely block the binding of B7-H7 protein to its receptor KIR3DL3.

TABLE 4
Blocking effect of antibodies against the binding of human
B7-H7 to human KIR3DL3 on the cell surface

| | Maximum | |
|---|---|---|
| Antibody | inhibition rate (%) | IC50 (nM) |
| PR003248 | 99.32 | 12.91 |
| PR003249 | 97.52 | 5.068 |
| PR003250 | 97.24 | 8.487 |
| PR003251 | 96.38 | 6.341 |
| PR003252 | 97.13 | 7.594 |
| PR003254 | 97.24 | 7.18 |
| PR003259 | 107.9 | 7.809 |
| PR003260 | 106 | 6.464 |
| PR003261 | 105.3 | 7.497 |
| PR003262 | 105 | 9.928 |
| PR003263 | 102.9 | 6.23 |
| PR003264 | 104.1 | 8.563 |
| PR003265 | 100.6 | 7.045 |
| PR003266 | 101.7 | 8.548 |
| PR003181 | 96.7 | 10.36 |
| PR004260 | 96.23 | 8.507 |
| PR004261 | 101.2 | 6.969 |
| PR004262 | 100.5 | 6.537 |
| PR004263 | 99.93 | 8.767 |

### Example 4.2. Blocking Effect of Antibodies Against the Binding of Human B7-H7 Protein to Human KIR3DL3 Protein

Human KIR3DL3 (Sino Biological, Cat #16055-H08H) was diluted with PBS to 5 μg/ml, added to a 96-well plate (Corning, cat #9018) at 100 μl per well, and incubated at 4° C. overnight. After the liquid was discarded, the plate was washed 3 times with a PBS buffer, 200 μl of PBS blocking solution containing 1% BSA was added, and the plate was incubated for 1 hour at room temperature. The blocking solution was discarded, and the plate was washed 3 times with a PBS buffer. Different concentrations of the antibodies to be tested were added along with 6 μg/ml biotin-B7H7 protein (Acro Biosystems, Cat #B77-H82F5) or Biotin-B7H4 protein (Acro Biosystems, Cat #B74-H82E2). The Biotin-B7H4 protein was a negative control protein that did not bind to KIR3DL3. The antibodies to be tested were subjected to 3-fold concentration dilution from 100 nM, with a total of 8 gradient concentrations. 100 μl of the diluted antibody was added to each well, and the plate was incubated at 4° C. overnight. After the plate was washed 3 times with a PBS buffer, StrepTactin-HRP Conjugate (Bio-rad, Cat #161-0380) diluted 500 times was added, and the plate was incubated at 37° C. for 1 hour in the dark. After the plate was washed 4 times with a PBS buffer, TMB (KPL SureBlue, Cat #5120-0077) was added at 100 μl/well, and the plate was placed at room temperature for about 10 minutes in the dark; and a stop solution (BBI life sciences, Cat #E661006-0200) was added to each well at 100 μl/well to stop the reaction, and the absorbance at 450 nm was determined in a microplate reader (PerkinElemer #Enspire).

The results are as shown in FIG. 6, indicating that B7-H7 protein can bind to KIR3DL3 protein in the absence of the antibodies, whereas B7-H4 protein does not bind to KIR3DL3 protein; and the antibodies PR004261, PR004262 and PR004263 to be tested can dose-dependently block the binding of B7-H7 protein to KIR3DL3 protein.

### Example 5. Determination of T Cell Activation Activity

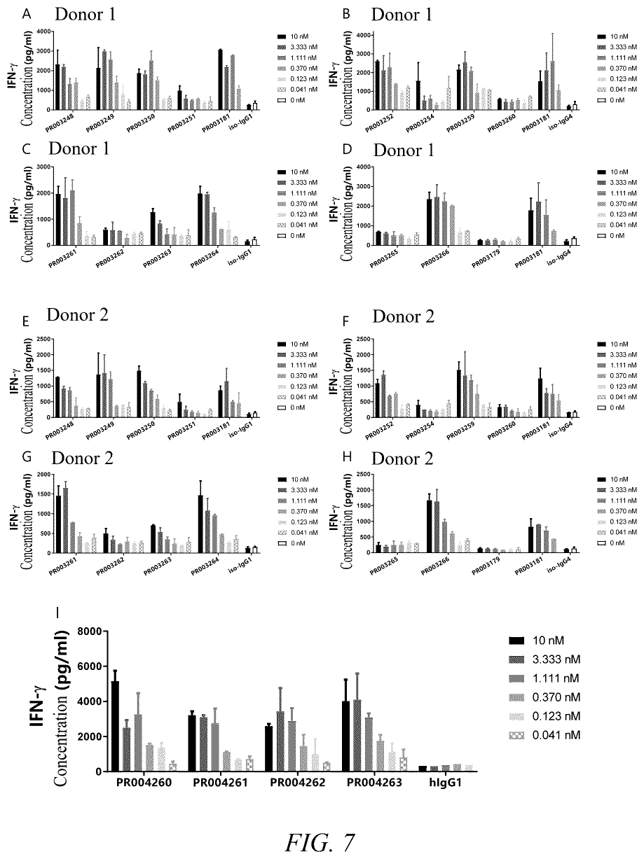

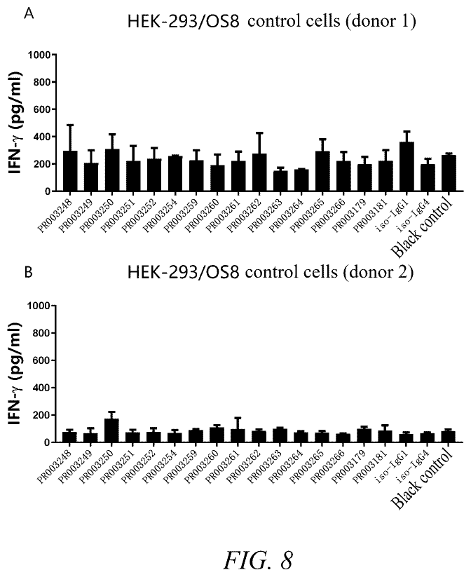

To detect the function of the anti-B7-H7 antibody in blocking T cell immune checkpoints and thus activating T cells, in this experiment, CHO-K1 cells overexpressing full-length B7-H7 and anti-human CD3 antibody OKT3 in the form of scFv were used as artificial antigen-presenting cells (CHO-K1/OS8/hB7-H7, KYinno Biotechnology Co., Ltd.) and negative control cells (CHO-K1/OS8, KYinno Biotechnology Co., Ltd.), respectively, T cells were isolated using a human T cell isolation kit (Miltenyi, #130-096-535) according to the method in the instructions, and the artificial antigen-presenting cells were co-cultured with the T cells, so as to detect the effect of the anti-B7-H7 antibody on T cell activation. Specifically, CHO-K1-OS8-hB7-H7 or control cells CHO-K1-OS8 were plated at a density of 1×10⁴ cells/well and cultured overnight. Human primary T cells were isolated and added to CHO-K1-OS8-hB7-H7 cells at a density of 2×10⁵ cells/100 μl/well. Subsequently, the antibody to be tested at 2× final concentration with 3-fold concentration gradient dilution was added at 100 μL/well, with the maximum final concentration of the antibody of 10 nM, a total of 6 concentrations for each antibody, two replicates set for each concentration. After culturing for 3 days, the supernatant was collected, and the concentration of IFN-γ was detected by ELISA. The results are as shown in FIG. 7. FIG. 7A-FIG. 7H show that the antibodies PR003248, PR003249, PR003250, PR003252, PR003259, PR003261, PR003264 and PR003266 and the control antibody have a strong activity of promoting T cell activation to secrete IFN-γ (where donor 1 in FIG. 7A-7D, and donor 2 in FIG. 7E-7H), and FIG. 71 shows that the antibodies PR004260, PR004261, PR004262 and PR004263 also have a strong activity of promoting T cells to secrete IFN-γ. If the artificial antigen-presenting cells do not express B7-H7, the antibodies do not show the activity of promoting T cell activation, indicating that the effect of the antibodies is B7-H7-specific (FIG. 8 shows that the anti-B7-H7 antibodies do not act on B7-H7 negative cells and thus cannot activate T cells).

### Example 6. Determination of NK Cell Killing Activity

To detect the function of the anti-B7-H7 antibody in activating NK cells, in this experiment, MDA-MB-231 cells overexpressing full-length B7-H7 were used as target cells (MDA-MB-231/hB7-H7, Harbour BioMed US, Inc.), NK cells were isolated using a human NK cell isolation kit (Miltenyi, #130-092-657) according to the method in the instructions, and the isolated and purified NK cells were activated with 100 IU/ml interleukin-2 for 2 days. The target cells MDA-MB-231/hB7-H7 were digested with trypsin and stained with 1 uM cell tracer dye CFSE at room temperature for 10 minutes in the dark. The activated NK cells were co-cultured with the target cells, the anti-B7-H7 antibody to be tested was added, and the cells were incubated in an incubator at 37° C. for 4 h. After the incubation was completed, 2 μl of 7AAD dead cell dye was added to each well, and the cells were uniformly mixed using a multi-channel pipette, and analyzed within 10 minutes on a flow cytometry sorter BD Cantoll to detect the killing on the target cells. (Killed target cells (%)=cells positive for both 7AAD staining and CFSE staining/cells positive for CFSE staining)

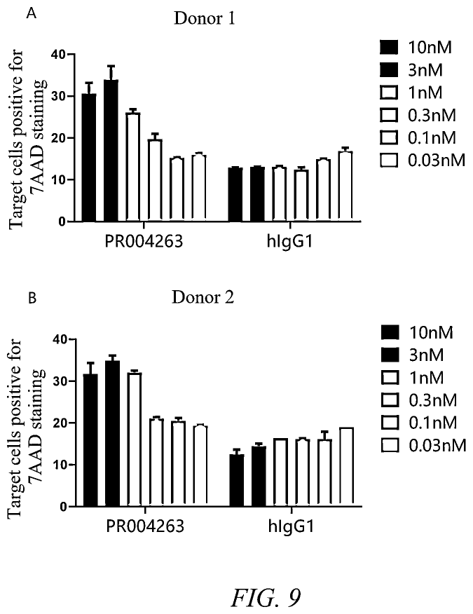

The results are as shown in FIG. 9, indicating that the antibody PR004263 significantly stimulates the NK cells to kill the target cells in a dose-dependent manner compared with the isotype control.

### Example 7. Determination of Affinity of Anti-B7-H7 Antibodies for Human B7-H7 Recombinant Protein

### Determination of Affinity by BLI

10× kinetics buffer (ForteBio, #18-1105) was diluted to 1× kinetics buffer for affinity testing and dilution of antigens and antibodies. The binding kinetics between antigen and antibody was analyzed using an Octet Red 96e (Fortebio) molecular interaction analyzer by biolayer interferometry (BLI) technology.

For determining the affinity of antibodies for antigens, the rotary speed of the sensor was set at 1000 rpm. AHC sensors (Fortebio, #18-5060) placed in one column were first equilibrated in a test buffer for 10 minutes, and then used to capture the anti-B7-H7 antibodies at a capture height of 0.7 nm; and after being equilibrated in the buffer for 120 s, the AHC sensors bound to human B7-H7 (concentrations: 50-3.125 nM and 0 nM) with 2-fold gradient dilution for 180 s and dissociated for 300 s. The AHC sensors were finally regenerated by immersing in a 10 mM glycine-hydrochloric acid solution with pH 1.5 to elute the protein bound to the sensors.

For data analysis using Octet Data Analysis software (Fortebio, version 11.0), 0 nM well was used as the reference well, reference subtraction was performed, “1:1 Global fitting” method was selected for data fitting, the kinetic parameters of the binding of the antigen to the antigen-binding protein were calculated, and the kₒₙ (1/Ms) value, k_{dis} (1/s) value and K_{D} (M) value were obtained. The results are as shown in Table 5.

TABLE 5
Binding affinity of anti-B7-H7 antibodies for human
B7-H7 protein (BLI)

| | Concentration | | | | |
|---|---|---|---|---|---|
| Antibody | (nM) | KD (M) | kon (1/Ms) | kdis (1/s) | Full R\hat{ }2 |
| PR003248 | 100 | 1.60E−09 | 8.80E+04 | 1.40E−04 | 0.9986 |
| PR003249 | 100 | 1.83E−09 | 9.70E+04 | 1.78E−04 | 0.9989 |
| PR003250 | 100 | 2.91E−09 | 9.32E+04 | 2.71E−04 | 0.998 |
| PR003251 | 100 | 6.70E−09 | 9.43E+04 | 6.32E−04 | 0.9969 |
| PR003252 | 100 | 1.06E−09 | 1.25E+05 | 1.32E−04 | 0.9975 |
| PR003254 | 100 | 3.23E−08 | 7.56E+04 | 2.45E−03 | 0.9742 |
| PR003259 | 100 | 1.64E−09 | 7.53E+04 | 1.24E−04 | 0.9984 |
| PR003260 | 100 | 1.68E−09 | 6.11E+04 | 1.02E−04 | 0.9989 |
| PR003261 | 100 | 4.60E−09 | 7.30E+04 | 3.36E−04 | 0.998 |
| PR003262 | 100 | 1.55E−09 | 7.55E+04 | 1.17E−04 | 0.9985 |
| PR003263 | 100 | 5.44E−09 | 8.11E+04 | 4.41E−04 | 0.9982 |
| PR003264 | 100 | 2.94E−09 | 3.90E+04 | 1.15E−04 | 0.998 |
| PR003265 | 100 | 5.95E−10 | 4.93E+04 | 2.93E−05 | 0.9994 |
| PR003266 | 100 | 9.93E−09 | 6.52E+04 | 6.48E−04 | 0.9985 |
| PR003181 | 100 | 2.63E−08 | 7.81E+04 | 2.05E−03 | 0.9737 |

### Example 8. Determination of Competition of Anti-B7-H7 Antibodies for Binding to B7-H7 Epitopes by BLI

Epitope competition experiments were performed on the anti-B7-H7 antibodies using the ForteBio Octet Red96e platform with the same experimental buffer as described above. First step, acquisition of 100% signal of the antibody: B7-H7 (Acro Biosystems, B77-H82F5) was captured using SA sensors (Fortebio, #18-5019) at a capture height of 0.25 nm. After being equilibrated in a buffer for 120 s, the sensors were immersed in various antibodies diluted to 100 nM for 240 s, and the final signal of the antibody binding to B7-H7 was recorded as the 100% signal for that antibody. Second step, epitope competition experiment: B7-H7 was captured using SA sensors at a capture height of 0.25 nm. The sensors were immersed in a first antibody (concentration: 100 nM) for 240 s, then the SA sensors were immersed in a mixture of the first antibody and a second antibody (final concentration of both antibodies: 100 nM) for 240 s, and the difference in signals before and after immersing the sensors in the mixture of the two antibodies was recorded as the signal for that antibody as the second antibody. The inhibition rate was calculated by the following equation,

$$Inhibition rate ( \% ) = ( A - B ) / A * 100$$

A: 100% signal (obtained from the first step) of a certain antibody, B: signal (obtained from the second step) of this antibody as the second antibody.

If the resulting inhibition rate is greater than 85 (%), it means that the epitopes to which the two antibodies bind completely overlap; and if the inhibition rate is less than 85 (%), it means that the epitopes to which the two antibodies bind do not completely overlap.

First, PR003249 was used as the first antibody, and the other antibodies were each used as the second antibody to detect the competition between the first antibody and the second antibody (PR003249 in the second antibody included 3 replicates). The results in Table 6-1 show that the second antibodies PR003248, PR003250, PR003252, PR003259, PR003261, PR003264, PR003265 and PR003266 completely compete with the first antibody PR003249, indicating that the first binding epitope to which they bind is the same; and the epitopes to which the control antibody PR003181 binds partially overlap with the first binding epitope. For the remaining antibodies that bind to other different epitopes, PR003251 was used as the first antibody, and the other antibodies were each used as the second antibody to detect the competition between the first antibody and the second antibody. The results in Table 6-2 show that PR003251, PR003254, PR003260, PR003262 and PR003263 completely compete with the first antibody, indicating that the second binding epitope to which they bind is the same; and the epitopes to which the control antibody PR003181 bind do not overlap with the second binding epitope. B7-H7 epitopes to which the antibodies bind are summarized in Tables 6-3.

TABLE 6-1
Competitive analysis of antibodies for binding to
B7-H7 in the first round

| | Competitive | First antibody |
|---|---|---|
| | inhibition rate (%) | PR003249 |
| Second | PR003249 | 106.54481 |
| antibody | PR003248 | 107.97038 |
| | PR003250 | 103.47287 |
| | PR003251 | −6.5446 |
| | PR003252 | 107.27535 |
| | PR003254 | −5.9583 |
| | PR003249 | 105.63873 |
| | PR003259 | 109.54106 |
| | PR003260 | −3.1776 |
| | PR003261 | 110.5473 |
| | PR003262 | −2.5972 |
| | PR003263 | −0.3244 |
| | PR003264 | 108.85338 |
| | PR003249 | 107.16221 |
| | PR003265 | 106.20329 |
| | PR003266 | 107.39825 |
| | PR003181 | 77.01487 |

TABLE 6-2
Competitive analysis of antibodies for binding to
B7-H7 in the second round

| | Competitive | First antibody |
|---|---|---|
| | inhibition rate (%) | PR003251 |
| Second | PR003251 | 97.890716 |
| antibody | PR003254 | 99.09022 |
| | PR003260 | 100.4908 |
| | PR003262 | 96.392817 |
| | PR003263 | 96.938776 |
| | PR003180 | 3.21033 |
| | PR003181 | 5.3217 |

TABLE 6-3
Summary of B7-H7 epitopes to which antibodies bind

| | |
|---|---|
| PR003249 PR003248 PR003250 PR003252 PR003259 | Epitope 1 |
| PR003261 PR003264 PR003265 PR003266 | |
| PR003181 | Epitope 1 |
| | (partially |
| | overlapped) |
| PR003251 PR003254 PR003260 PR003262 PR003263 | Epitope 2 |

### Example 9. Cross-Reactivity to Other Members of the B7 Family

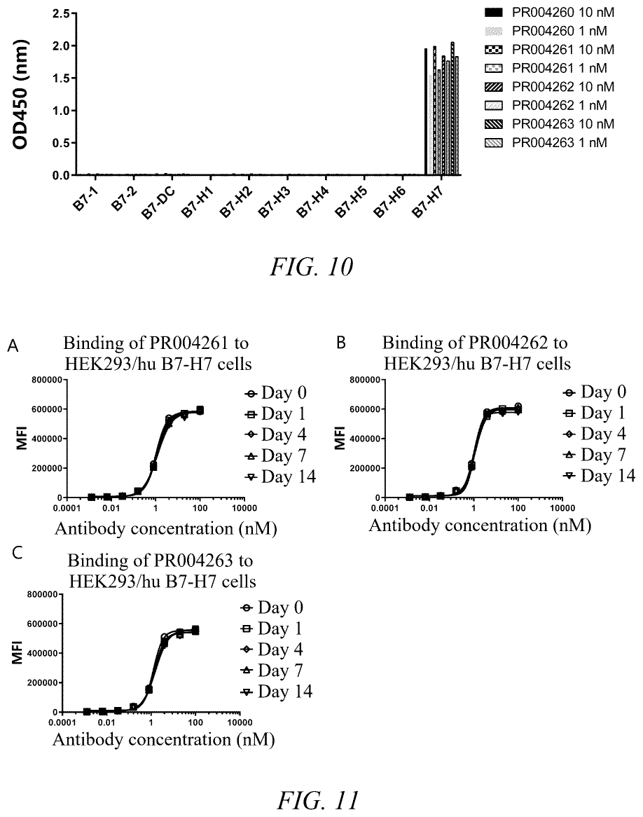

B7 family proteins (see Table 7 for details) were respectively diluted to 1 g/mL with PBS, added into a 96-well plate (Corning, #9018) at 100 μl/well, and incubated at 4° C. overnight. After the liquid was discarded, the plate was washed 3 times with a PBST buffer (pH 7.4, containing 0.05% tween-20), 250 μl of blocking solution containing 2% BSA was added, and the plate was incubated for 1 hour at 37° C. The blocking solution was discarded, the plate was washed 3 times with PBST buffer (pH 7.4, containing 0.05% Tween-20), the antigen-binding protein to be tested was diluted to 2 concentrations of 10 nM and 1 nM, 100 μl of the diluted antigen-binding protein was added to each well, and the plate was incubated for 1 hour at 37° C., with the isotype antibody as the control. After the plate was washed 3 times with PBST buffer (pH 7.4, containing 0.05% Tween-20), goat anti-human HRP secondary antibody (Invitrogen, #A18805) diluted 5000 times was added, and the plate was incubated at 37° C. for 1 hour in the dark. After the plate was washed 3 times with PBST buffer (pH 7.4, containing 0.05% tween-20), TMB (Biopanda, #TMB-S-003) was added at 100 μl/well, and the plate was placed at room temperature for about 30 minutes in the dark; and a stop solution (BBI life sciences, #E661006-0200) was added to each well at 50 μl/well to stop the reaction, and the absorbance at 450 nm (OD450) was determined in a microplate reader (PerkinElemer, #Enspire). FIG. 10 shows that the antibodies of the present invention only specifically binds to B7-H7 protein without cross-reacting with other member proteins of the B7 family.

TABLE 7
Material information of B7 family proteins used in this example

| Other members of the B7 family | Supplier | Catalog No. |
|---|---|---|
| Human B7-1/CD80 protein, | Acro | B71-H5259 |
| Fc tag (HPLC validated) | | |
| Human B7-2/CD86 protein, Fc tag | Acro | CD6-H5257 |
| Human B7-DC/PD-L2/CD273 protein, | Sino Biological | H10292-H02H |
| recombinant (Fc tag) | | |
| Human PD-L1/B7-H1 protein, | Acro | PD1-H5229 |
| His tag (HPLC validated) | | |
| CD275/ICOS ligand protein, | Sino Biological | 11559-H02H |
| human, recombinant (Fc tag) | | |
| B7-H3/CD276 protein, human, | Sino Biological | 11188-H02H |
| recombinant (ECD, Fc tag) | | |
| Recombinant human B7H4 Fc chimera | R&D | 8870-B7-050 |
| Recombinant human | R&D | 7126-B7-050 |
| VISTA/B7H5/PD-1H Fc chimera | | |
| B7-H6 /NCR3LG1 protein, | Sino Biological | 16140-H02H |
| human, recombinant (Fc tag) | | |
| Recombinant human | R&D | 8084-B7-050 |
| B7-H7/HHLA2 Fc chimera | | |

### Example 10. Analysis of Stability in Sera

30 μl of the antibody was taken and diluted with 270 μl of normal human sera (concentration in serum: 90%), the diluted antibody was divided into 5 parts, the 5 parts were incubated at 37° C. for 0, 1, 4, 7 and 14 days, respectively, then taken out, snap-frozen with liquid nitrogen, and stored at −80° C. The binding of the antibody to B7-H7 on HEK-293/hB7-H7 cells was detected by flow cytometry. The HEK-293/hB7-H7 cells were digested and resuspended in PBS containing 2% BSA. The cells were adjusted to a cell density of 1×10⁶ cells/mL, respectively. The cells were seeded in a 96-well V-bottom plate (Corning, #3894) at 100 μL/well, and the antibody to be tested at 2× final concentration with 3-fold concentration gradient dilution was then added at 100 μL/well. The cells were incubated at 4° C. for 2 hours in the dark. After the incubation, the cells were rinsed twice by adding pre-chilled PBS containing 2% BSA at 100 μL/well, and centrifuged at 500 g at 4° C. for 5 minutes, and the supernatant was discarded. A fluorescent secondary antibody (Alexa Fluor 488-conjugated AffiniPure Goat Anti-Human IgG, Fcγ Fragment Specific, Jackson, #109-545-098, 1:500 dilution) was added at 100 μL/well, and the cells were incubated at 4° C. for 60 minutes in the dark. The cells were washed twice by adding pre-chilled PBS containing 2% BSA at 100 μL/well, and centrifuged at 500 g at 4° C. for 5 minutes, and the supernatant was discarded. Finally, the cells were resuspended in pre-chilled PBS containing 2% BSA at 200 μL/well, and the fluorescence signal values were read using an ACEA Novocyte3000 flow cytometer. The results in FIG. 11 show that PR004261, PR004262 and PR004263 have good stability in human sera at 37° C. for 14 days.

### Example 11. Anti-Tumor Efficacy of Anti-B7-H7 Antibodies NCG Mouse MDA-MB-231 Tumor Model Having Reconstituted Human PBMC Immune System

On the day of cell inoculation, each NCG mouse was subcutaneously inoculated with 5×10⁶ MDA-MB-231 tumor cells (MDA-MB-231/hB7-H7 cells) overexpressing human B7-H7, wherein the cells were resuspended in a PBS and Matrigel (1:1) mixed solution (0.1 mL/mouse), and subcutaneous inoculation was performed. The mice were grouped when the mean tumor volume was about 90 mm³, 24 mice were divided into 4 groups, and each mouse was intravenously inoculated with 5×10⁶ human PBMCs, wherein the cells were resuspended in 200 μl of PBS. The administration was started on the next day, the administration cycle was 4 weeks in total with an administration frequency of twice a week, tail vein administration was used, and the efficacy of the antibodies PR004261, PR004262 and PR004263 at a dose of 20 MPK was tested. After the administration was started, the body weight was weighed and the tumor volume was measured twice a week. The tumor volume was calculated as follows: Tumor volume (mm³)=0.5× tumor long diameter×tumor short diameter². Experimental observations were ended on day 36 after administration, and all mice were subsequently euthanized.

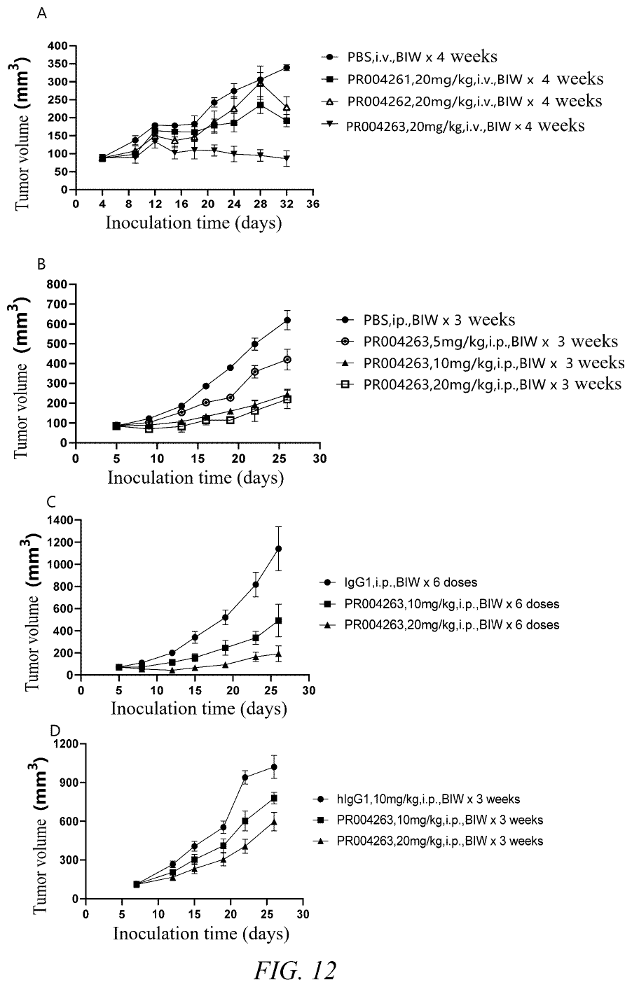

For the in vivo anti-tumor effects in the NCG mouse MDA-MB-231/hB7-H7 tumor model having reconstituted human PBMC immune system, see FIG. 12A, and specifically, the mean tumor volume of the mice in the vehicle control group on day 32 after administration is 339 mm³. The mean tumor volume in the treatment group using the test drug PR004261 (20 mg/kg) on day 32 after administration is 192 mm³, which is significantly different (p value<0.0001) from that in the vehicle control group, with the tumor inhibition rate TGI (%) of 43.43%. The mean tumor volume in the treatment group using the test drug PR004262 (20 mg/kg) on day 32 after administration is 229 mm³, which is significantly different (p value of 0.0049) from that in the vehicle control group, with the tumor inhibition rate TGI (%) of 32.52%. The mean tumor volume in the treatment group using the test drug PR004263 (20 mg/kg) on day 32 after administration is 86 mm³, which is significantly different (p value<0.0001) from that in the vehicle control group, with the tumor inhibition rate TGI (%) of 74.57%.

In the second experiment, a similar tumor inoculation regimen was performed, the administration cycle was a 3 weeks in total with an administration frequency of twice a week, intraperitoneal administration was used, and the efficacy of the antibody PR004263 at 3 doses of 5 MPK, 10 MPK and 20 MPK was tested. After the administration was started, the body weight was weighed and the tumor volume was measured twice a week. The tumor volume was calculated as follows: Tumor volume (mm³)=0.5× tumor long diameter×tumor short diameter². Experimental observations were ended on day 36 after administration, and all mice were subsequently euthanized.

For the in vivo anti-tumor effect in the tumor models, see FIG. 12B, and specifically, the mean tumor volume of the mice in the vehicle control group on day 26 after inoculation is 618 mm³. The mean tumor volume in the treatment group using the test drug PR004263 (5 mg/kg) on day 26 after inoculation is 420 mm³, which is significantly different (p value<0.0198) from that in the vehicle control group, with the tumor inhibition rate TGI (%) of 32.1%. The mean tumor volume in the treatment group using the test drug PR004263 (10 mg/kg) on day 26 after inoculation is 243 mm³, which is significantly different (p value of 0.0001) from that in the vehicle control group, with the tumor inhibition rate TGI (%) of 60.65%. The mean tumor volume in the treatment group using the test drug PR004263 (20 mg/kg) on day 26 after inoculation is 219 mm³, which is significantly different (p value<0.0001) from that in the vehicle control group, with the tumor inhibition rate TGI (%) of 64.52%.

### NCG Mouse NUGC-4 Tumor Model Having Reconstituted Human PBMC Immune System and NCG Mouse HCC-827 Tumor Model Having Reconstituted Human PBMC Immune System

Both NUGC-4 tumor cells and HCC-827 tumor cells express B7-H7 endogenously. A NCG mouse NUGC-4 tumor model having reconstituted human PBMC immune system was used for in vivo pharmacodynamic studies. The specific method was as follows: on the day of cell inoculation, each NCG mouse was subcutaneously co-inoculated with 5×10⁶ NUGC-4 tumor cells and 1×10⁶ human PBMCs, i.e., subcutaneous inoculation of mixed cells. When the mean tumor volume of the mice in each group was 70 mm³, grouping and administration were performed, with a total of 6 administrations via intraperitoneal injection. After the administration was started, the body weight was weighed and the tumor volume was measured twice a week. The tumor volume was calculated as follows: Tumor volume (mm³)=0.5× tumor long diameter×tumor short diameter². Data analysis was performed using t-test. For the in vivo anti-tumor effects in the NCG mouse NUGC-4 tumor model having reconstituted human PBMC immune system, see FIG. 12C, and specifically, the mean tumor volume of the mice in the antibody control group on day 26 after administration is 1140.62 mm³. The mean tumor volume in the treatment group using the test drug PR004263 (10 mg/kg) on day 26 after administration is 491.38 mm³, which is significantly different (p value<0.05) from that in the vehicle control group, with the tumor inhibition rate TGI (%) of 56.92%. The mean tumor volume in the treatment group at 20 mg/kg on day 26 after administration is 191.99 mm³, which is significantly different (p value<0.01) from that in the vehicle control group, with the tumor inhibition rate TGI (%) of 83.17%.

A NCG mouse HCC-827 tumor model having reconstituted human PBMC immune system was used for in vivo pharmacodynamic studies. The specific method was as follows: on the day of cell inoculation, each NCG mouse was subcutaneously inoculated with 1×10⁷ HCC-827 cells. The mice were grouped when the mean tumor volume was about 110 mm³, each mouse was intravenously inoculated with 5×10⁶ human PBMCs, and the administration was started on the next day. The administration cycle was 5 administrations with an administration frequency of twice a week, and the mode of administration was intraperitoneal injection. After the administration was started, the body weight was weighed and the tumor volume was measured twice a week. The tumor volume was calculated as follows: Tumor volume (mm³)=0.5× tumor long diameter×tumor short diameter². Experimental observations were ended on day 26 after administration, and all mice were subsequently euthanized. For the in vivo anti-tumor effects in the NCG mouse HCC-827 tumor model having reconstituted human PBMC immune system, see FIG. 12D, and specifically, the mean tumor volume of the mice in the antibody control group on day 26 after administration is 1020.74 mm³. The mean tumor volume in the treatment group using the test drug PR004263 (10 mg/kg) on day 26 after administration is 778.35 mm³, which is significantly different (p value<0.05) from that in the vehicle control group, with the tumor inhibition rate TGI (%) of 23.75%. The mean tumor volume in the treatment group at 20 mg/kg on day 26 after administration is 597.69 mm³, which is significantly different (p value<0.01) from that in the vehicle control group, with the tumor inhibition rate TGI (%) of 41.45%.

1. An anti-B7-H7 antibody or an antigen-binding fragment thereof, characterized in that the antibody or the antigen-binding fragment thereof comprises a light chain variable region (VL) and a heavy chain variable region (VH); wherein
the VL comprises LCDR1, LCDR2 and LCDR3 having amino acid sequences set forth in SEQ ID NOs: 87, 97 and 120, respectively, and the VH comprises HCDR1, HCDR2 and HCDR3 having amino acid sequences set forth in SEQ ID NOs: 12, 34 and 66, respectively; or
the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 28 and 58, respectively; or
the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 37 and 58, respectively; or
the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 37 and 58, respectively; or
the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 37 and 58, respectively; or
the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 38 and 58, respectively; or
the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 38 and 58, respectively; or
the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 38 and 58, respectively; or
the VL comprises LCDR1, LCDR2 and LCDR3 having amino acid sequences set forth in SEQ ID NOs: 82, 94 and 122, respectively, and the VH comprises HCDR1, HCDR2 and HCDR3 having amino acid sequences set forth in SEQ ID NOs: 7, 28 and 58, respectively; or
the VL comprises LCDR1, LCDR2 and LCDR3 having amino acid sequences set forth in SEQ ID NOs: 82, 94 and 123, respectively, and the VH comprises HCDR1, HCDR2 and HCDR3 having amino acid sequences set forth in SEQ ID NOs: 7, 28 and 58, respectively; or
the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 27 and 63, respectively; or
the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 39 and 63, respectively; or
the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 39 and 63, respectively; or
the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 39 and 63, respectively; or
the VL comprises LCDR1, LCDR2 and LCDR3 having amino acid sequences set forth in SEQ ID NOs: 85, 97 and 116, respectively, and the VH comprises HCDR1, HCDR2 and HCDR3 having amino acid sequences set forth in SEQ ID NOs: 9, 40 and 63, respectively; or
the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 40 and 63, respectively; or
the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 40 and 63, respectively; or
the VL comprises LCDR1, LCDR2 and LCDR3 having amino acid sequences set forth in SEQ ID NOs: 85, 97 and 124, respectively, and the VH comprises HCDR1, HCDR2 and HCDR3 having amino acid sequences set forth in SEQ ID NOs: 9, 27 and 63, respectively; or
the VL comprises LCDR1, LCDR2 and LCDR3 having amino acid sequences set forth in SEQ ID NOs: 85, 97 and 125, respectively, and the VH comprises HCDR1, HCDR2 and HCDR3 having amino acid sequences set forth in SEQ ID NOs: 9, 27 and 63, respectively; or
the VL comprises LCDR1, LCDR2 and LCDR3 having amino acid sequences set forth in SEQ ID NOs: 81, 94 and 111, respectively, and the VH comprises HCDR1, HCDR2 and HCDR3 having amino acid sequences set forth in SEQ ID NOs: 7, 27 and 57, respectively; or
the VL comprises LCDR1, LCDR2 and LCDR3 having amino acid sequences set forth in SEQ ID NOs: 82, 94 and 112, respectively, and the VH comprises HCDR1, HCDR2 and HCDR3 having amino acid sequences set forth in SEQ ID NOs: 7, 28 and 59, respectively; or
the VL comprises LCDR1, LCDR2 and LCDR3 having amino acid sequences set forth in SEQ ID NOs: 83, 95 and 113, respectively, and the VH comprises HCDR1, HCDR2 and HCDR3 having amino acid sequences set forth in SEQ ID NOs: 8, 29 and 60, respectively; or
the VL comprises LCDR1, LCDR2 and LCDR3 having amino acid sequences set forth in SEQ ID NOs: 82, 96 and 114, respectively, and the VH comprises HCDR1, HCDR2 and HCDR3 having amino acid sequences set forth in SEQ ID NOs: 9, 27 and 61, respectively; or
the VL comprises LCDR1, LCDR2 and LCDR3 having amino acid sequences set forth in SEQ ID NOs: 84, 95 and 115, respectively, and the VH comprises HCDR1, HCDR2 and HCDR3 having amino acid sequences set forth in SEQ ID NOs: 10, 29 and 62, respectively; or
the VL comprises LCDR1, LCDR2 and LCDR3 having amino acid sequences set forth in SEQ ID NOs: 83, 98 and 113, respectively, and the VH comprises HCDR1, HCDR2 and HCDR3 having amino acid sequences set forth in SEQ ID NOs: 8, 30 and 62, respectively; or
the VL comprises LCDR1, LCDR2 and LCDR3 having amino acid sequences set forth in SEQ ID NOs: 82, 94 and 117, respectively, and the VH comprises HCDR1, HCDR2 and HCDR3 having amino acid sequences set forth in SEQ ID NOs: 9, 31 and 64, respectively; or
the VL comprises LCDR1, LCDR2 and LCDR3 having amino acid sequences set forth in SEQ ID NOs: 86, 95 and 118, respectively, and the VH comprises HCDR1, HCDR2 and HCDR3 having amino acid sequences set forth in SEQ ID NOs: 8, 32 and 62, respectively; or
the VL comprises LCDR1, LCDR2 and LCDR3 having amino acid sequences set forth in SEQ ID NOs: 83, 99 and 119, respectively, and the VH comprises HCDR1, HCDR2 and HCDR3 having amino acid sequences set forth in SEQ ID NOs: 11, 33 and 65, respectively; or
the VL comprises LCDR1, LCDR2 and LCDR3 having amino acid sequences set forth in SEQ ID NOs: 88, 100 and 121, respectively, and the VH comprises HCDR1, HCDR2 and HCDR3 having amino acid sequences set forth in SEQ ID NOs: 13, 35 and 67, respectively; or
the VL comprises LCDR1, LCDR2 and LCDR3 having amino acid sequences set forth in SEQ ID NOs: 82, 94 and 111, respectively, and the VH comprises HCDR1, HCDR2 and HCDR3 having amino acid sequences set forth in SEQ ID NOs: 7, 36 and 68, respectively.

2-3. (canceled)

4. The anti-B7-H7 antibody or the antigen-binding fragment thereof of claim 1, wherein
the VL comprises an amino acid sequence set forth in SEQ ID NO: 161, and the VH comprises an amino acid sequence set forth in SEQ ID NO: 143; or
the VL comprises an amino acid sequence set forth in SEQ ID NO: 152, and the VH comprises an amino acid sequence set forth in SEQ ID NO: 133; or
the VL comprises an amino acid sequence set forth in SEQ ID NO: 152, and the VH comprises an amino acid sequence set forth in SEQ ID NO: 146; or
the VL comprises an amino acid sequence set forth in SEQ ID NO: 164, and the VH comprises an amino acid sequence set forth in SEQ ID NO: 146; or
the VL comprises an amino acid sequence set forth in SEQ ID NO: 165, and the VH comprises an amino acid sequence set forth in SEQ ID NO: 146; or
the VL comprises an amino acid sequence set forth in SEQ ID NO: 152, and the VH comprises an amino acid sequence set forth in SEQ ID NO: 147; or
the VL comprises an amino acid sequence set forth in SEQ ID NO: 164, and the VH comprises an amino acid sequence set forth in SEQ ID NO: 147; or
the VL comprises an amino acid sequence set forth in SEQ ID NO: 165, and the VH comprises an amino acid sequence set forth in SEQ ID NO: 147; or
the VL comprises an amino acid sequence set forth in SEQ ID NO: 164, and the VH comprises an amino acid sequence set forth in SEQ ID NO: 133; or
the VL comprises an amino acid sequence set forth in SEQ ID NO: 165, and the VH comprises an amino acid sequence set forth in SEQ ID NO: 133; or
the VL comprises an amino acid sequence set forth in SEQ ID NO: 156, and the VH comprises an amino acid sequence set forth in SEQ ID NO: 138; or
the VL comprises an amino acid sequence set forth in SEQ ID NO: 156, and the VH comprises an amino acid sequence set forth in SEQ ID NO: 148; or
the VL comprises an amino acid sequence set forth in SEQ ID NO: 166, and the VH comprises an amino acid sequence set forth in SEQ ID NO: 148; or
the VL comprises an amino acid sequence set forth in SEQ ID NO: 167, and the VH comprises an amino acid sequence set forth in SEQ ID NO: 148; or
the VL comprises an amino acid sequence set forth in SEQ ID NO: 156, and the VH comprises an amino acid sequence set forth in SEQ ID NO: 149; or
the VL comprises an amino acid sequence set forth in SEQ ID NO: 166, and the VH comprises an amino acid sequence set forth in SEQ ID NO: 149; or
the VL comprises an amino acid sequence set forth in SEQ ID NO: 167, and the VH comprises an amino acid sequence set forth in SEQ ID NO: 149; or
the VL comprises an amino acid sequence set forth in SEQ ID NO: 166, and the VH comprises an amino acid sequence set forth in SEQ ID NO: 138; or
the VL comprises an amino acid sequence set forth in SEQ ID NO: 167, and the VH comprises an amino acid sequence set forth in SEQ ID NO: 138; or
the VL comprises an amino acid sequence set forth in SEQ ID NO: 151, and the VH comprises an amino acid sequence set forth in SEQ ID NO: 132; or
the VL comprises an amino acid sequence set forth in SEQ ID NO: 152, and the VH comprises an amino acid sequence set forth in SEQ ID NO: 134; or
the VL comprises an amino acid sequence set forth in SEQ ID NO: 153, and the VH comprises an amino acid sequence set forth in SEQ ID NO: 135; or
the VL comprises an amino acid sequence set forth in SEQ ID NO: 154, and the VH comprises an amino acid sequence set forth in SEQ ID NO: 136; or
the VL comprises an amino acid sequence set forth in SEQ ID NO: 155, and the VH comprises an amino acid sequence set forth in SEQ ID NO: 137; or
the VL comprises an amino acid sequence set forth in SEQ ID NO: 157, and the VH comprises an amino acid sequence set forth in SEQ ID NO: 139; or
the VL comprises an amino acid sequence set forth in SEQ ID NO: 158, and the VH comprises an amino acid sequence set forth in SEQ ID NO: 140; or
the VL comprises an amino acid sequence set forth in SEQ ID NO: 159, and the VH comprises an amino acid sequence set forth in SEQ ID NO: 141; or
the VL comprises an amino acid sequence set forth in SEQ ID NO: 160, and the VH comprises an amino acid sequence set forth in SEQ ID NO: 142; or
the VL comprises an amino acid sequence set forth in SEQ ID NO: 162, and the VH comprises an amino acid sequence set forth in SEQ ID NO: 144; or
the VL comprises an amino acid sequence set forth in SEQ ID NO: 163, and the VH comprises an amino acid sequence set forth in SEQ ID NO: 145.

5. The anti-B7-H7 antibody or the antigen-binding fragment thereof of claim 1, characterized in that the anti-B7-H7 antibody or the antigen-binding fragment thereof is a full-length antibody, Fab, Fab′, F(ab′)₂, Fv, a bispecific antibody, a multispecific antibody, or a monoclonal antibody.

6. The anti-B7-H7 antibody or the antigen-binding fragment thereof of claim 5, characterized in that the anti-B7-H7 antibody is a full-length antibody comprising an antibody heavy chain constant region and an antibody light chain constant region
, wherein the heavy chain constant region is selected from hIgG1, hIgG2, hIgG3 or hIgG4, and the light chain constant region is selected from a kappa chain or a lamda chain of a human antibody.

7. The anti-B7-H7 antibody or the antigen-binding fragment thereof of claim 6, characterized in that the full-length antibody comprises a heavy chain and a light chain;
wherein,
the heavy chain comprises an amino acid sequence set forth in SEQ ID NO: 186, and the light chain comprises an amino acid sequence set forth in SEQ ID NO: 202; or
the heavy chain comprises an amino acid sequence set forth in SEQ ID NO: 180, and the light chain comprises an amino acid sequence set forth in SEQ ID NO: 202; or
the heavy chain comprises an amino acid sequence set forth in SEQ ID NO: 184, and the light chain comprises an amino acid sequence set forth in SEQ ID NO: 193; or
the heavy chain comprises an amino acid sequence set forth in SEQ ID NO: 170, and the light chain comprises an amino acid sequence set forth in SEQ ID NO: 193; or
the heavy chain comprises an amino acid sequence set forth in SEQ ID NO: 187, and the light chain comprises an amino acid sequence set forth in SEQ ID NO: 193; or
the heavy chain comprises an amino acid sequence set forth in SEQ ID NO: 187, and the light chain comprises an amino acid sequence set forth in SEQ ID NO: 205; or
the heavy chain comprises an amino acid sequence set forth in SEQ ID NO: 187, and the light chain comprises an amino acid sequence set forth in SEQ ID NO: 206; or
the heavy chain comprises an amino acid sequence set forth in SEQ ID NO: 188, and the light chain comprises an amino acid sequence set forth in SEQ ID NO: 193; or
the heavy chain comprises an amino acid sequence set forth in SEQ ID NO: 188, and the light chain comprises an amino acid sequence set forth in SEQ ID NO: 205; or
the heavy chain comprises an amino acid sequence set forth in SEQ ID NO: 188, and the light chain comprises an amino acid sequence set forth in SEQ ID NO: 206; or
the heavy chain comprises an amino acid sequence set forth in SEQ ID NO: 184, and the light chain comprises an amino acid sequence set forth in SEQ ID NO: 205; or
the heavy chain comprises an amino acid sequence set forth in SEQ ID NO: 184, and the light chain comprises an amino acid sequence set forth in SEQ ID NO: 206; or
the heavy chain comprises an amino acid sequence set forth in SEQ ID NO: 185, and the light chain comprises an amino acid sequence set forth in SEQ ID NO: 197; or
the heavy chain comprises an amino acid sequence set forth in SEQ ID NO: 175, and the light chain comprises an amino acid sequence set forth in SEQ ID NO: 197; or
the heavy chain comprises an amino acid sequence set forth in SEQ ID NO: 189, and the light chain comprises an amino acid sequence set forth in SEQ ID NO: 197; or
the heavy chain comprises an amino acid sequence set forth in SEQ ID NO: 189, and the light chain comprises an amino acid sequence set forth in SEQ ID NO: 207; or
the heavy chain comprises an amino acid sequence set forth in SEQ ID NO: 189, and the light chain comprises an amino acid sequence set forth in SEQ ID NO: 208; or
the heavy chain comprises an amino acid sequence set forth in SEQ ID NO: 190, and the light chain comprises an amino acid sequence set forth in SEQ ID NO: 197; or
the heavy chain comprises an amino acid sequence set forth in SEQ ID NO: 190, and the light chain comprises an amino acid sequence set forth in SEQ ID NO: 207; or
the heavy chain comprises an amino acid sequence set forth in SEQ ID NO: 190, and the light chain comprises an amino acid sequence set forth in SEQ ID NO: 208; or
the heavy chain comprises an amino acid sequence set forth in SEQ ID NO: 185, and the light chain comprises an amino acid sequence set forth in SEQ ID NO: 207; or
the heavy chain comprises an amino acid sequence set forth in SEQ ID NO: 185, and the light chain comprises an amino acid sequence set forth in SEQ ID NO: 208; or
the heavy chain comprises an amino acid sequence set forth in SEQ ID NO: 169, and the light chain comprises an amino acid sequence set forth in SEQ ID NO: 192; or
the heavy chain comprises an amino acid sequence set forth in SEQ ID NO: 171, and the light chain comprises an amino acid sequence set forth in SEQ ID NO: 193; or
the heavy chain comprises an amino acid sequence set forth in SEQ ID NO: 172, and the light chain comprises an amino acid sequence set forth in SEQ ID NO: 194; or
the heavy chain comprises an amino acid sequence set forth in SEQ ID NO: 173, and the light chain comprises an amino acid sequence set forth in SEQ ID NO: 195; or
the heavy chain comprises an amino acid sequence set forth in SEQ ID NO: 174, and the light chain comprises an amino acid sequence set forth in SEQ ID NO: 196; or
the heavy chain comprises an amino acid sequence set forth in SEQ ID NO: 176, and the light chain comprises an amino acid sequence set forth in SEQ ID NO: 198; or
the heavy chain comprises an amino acid sequence set forth in SEQ ID NO: 177, and the light chain comprises an amino acid sequence set forth in SEQ ID NO: 199; or
the heavy chain comprises an amino acid sequence set forth in SEQ ID NO: 178, and the light chain comprises an amino acid sequence set forth in SEQ ID NO: 200; or
the heavy chain comprises an amino acid sequence set forth in SEQ ID NO: 179, and the light chain comprises an amino acid sequence set forth in SEQ ID NO: 201; or
the heavy chain comprises an amino acid sequence set forth in SEQ ID NO: 181, and the light chain comprises an amino acid sequence set forth in SEQ ID NO: 203; or
the heavy chain comprises an amino acid sequence set forth in SEQ ID NO: 182, and the light chain comprises an amino acid sequence set forth in SEQ ID NO: 204.

8. An isolated nucleic acid, characterized by encoding the anti-B7-H7 antibody or the antigen-binding fragment thereof of claim 1.

9. A recombinant expression vector, characterized by comprising the isolated nucleic acid of claim 8.

10. A transformant, characterized by comprising the recombinant expression vector of claim 9.

11. A chimeric antigen receptor, characterized by comprising the anti-B7-H7 antibody or the antigen-binding fragment thereof of claim 1.

12. A genetically modified cell, characterized in that the genetically modified cell comprises the chimeric antigen receptor of claim 11.

13. (canceled)

14. An antibody-drug conjugate, characterized by comprising an antibody moiety and a conjugated moiety, wherein the antibody moiety comprises the anti-B7-H7 antibody or the antigen-binding fragment thereof of claim 1, the conjugated moiety comprises a detectable marker, a drug, a toxin, a cytokine, a radionuclide, an enzyme, or a combination thereof, and the antibody moiety is conjugated to the conjugated moiety via a chemical bond or a linker.

15. A pharmaceutical composition, characterized by comprising the anti-B7-H7 antibody or the antigen-binding fragment thereof of claim 1.

16. (canceled)

17. A method for treating and/or preventing a cancer, comprising administering to a subject in need thereof the anti-B7-H7 antibody or the antigen-binding fragment thereof of claim 1.

18. (canceled)

19. A method for detecting B7-H7, the method comprising the step of performing detection using the anti-B7-H7 antibody or the antigen-binding fragment thereof of claim 1.

20. A method for preventing and/or treating a B7-H7-related disease or disorder in a subject in need thereof, method comprising the step of administering to the subject the anti-B7-H7 antibody or the antigen-binding fragment thereof of claim 1.

21. The method of claim 20, wherein the disease or disorder is a cancer.

22. The genetically modified cell of claim 12, which is an immune cell.

23. The genetically modified cell of claim 22, wherein the immune cell is a T cell or an NK cell.

24. The pharmaceutical composition of claim 15, which further comprises a combined therapeutic agent, and the combined therapeutic agent is selected from the group consisting of a chemotherapeutic agent, a radiotherapeutic agent, an immunosuppressant, and a cytotoxic drug.

25. The method of claim 21, wherein the cancer is selected from the group consisting of lung cancer, breast cancer, gastric cancer, small intestine cancer, colon cancer, rectal cancer, pancreatic cancer, kidney cancer, bladder cancer, and osteosarcoma.
